(19) United States
(12) Patent Application Publication
(10) Pub. No.: US 2016/0285008 A1
(43) Pub. Date: Sep. 29, 2016

(54) ORGANIC ELECTROLUMINESCENT ELEMENT, DISPLAY DEVICE, AND LIGHTING DEVICE

(71) Applicant: KONICA MINOLTA, INC., Tokyo (JP)

(72) Inventors: Yasushi OKUBO, Hino-shi, Tokyo (JP); Keiko ISHIDAI, Hachioji-shi, Tokyo (JP); Takeshi HAKII, Sagamihara-shi, Kanagawa (JP)

(21) Appl. No.: 15/031,921

(22) PCT Filed: Oct. 28, 2014
(86) PCT No.: PCT/JP2014/078573

§ 371 Date: Apr. 25, 2016

(30) Foreign Application Priority Data
Nov. 1, 2013 (JP) 2013-228226

(51) Int. Cl. H01L 51/00; C07D 401/14; C07D 213/06; C07D 471/04; C08G 61/12

(52) U.S. Cl. CPC H01L 51/0072; H01L 51/0073; H01L 51/0067; C07D 471/04; C08G 61/12; H01L 51/0039; C07D 213/06; C07D 401/14; H01L 51/0056; C08G 61/124; H01L 51/0035; H01L 2251/303; C08G 2261/95; C08G 2261/514; C08G 2261/3142; C08G 2261/1434; H01L 51/5092

(57) ABSTRACT

An object of the present invention is to provide an organic electroluminescent element which has a high luminous efficiency and an excellent driving voltage and exhibits excellent stability, and a display device and a lighting device that are equipped with the organic electroluminescent element.

The organic electroluminescent element of the present invention is an organic electroluminescent element which includes at least an electron injection layer, an electron transport layer, and a luminous layer between a positive electrode and a negative electrode and in which the electron injection layer contains an electride, the electron transport layer contains an organic compound having a nitrogen atom, at least one of the nitrogen atoms has a lone pair of electrons that does not participate in aromaticity, and the lone pair of electrons does not coordinate a metal.

### TECHNICAL FIELD

The present invention relates to an organic electroluminescent element, a display device, and a lighting device. More specifically, it relates to an organic electroluminescent element improved in driving voltage and stability by the use of an electride.

### BACKGROUND ART

An organic electroluminescent element (hereinafter, also referred to as the “organic EL element”) is a thin film type all-solid-state element constituted by interposing an organic thin film layer (single layer portion or multilayer portion) containing an organic luminescent substance between the positive electrode and the negative electrode. An electron and a hole are injected to the organic thin film layer (hereinafter, also referred to as the organic layer) from the negative electrode and the positive electrode, respectively, when a voltage is applied to such an organic EL element, and these are rebound in the luminous layer (organic luminescent substance containing layer) to generate an exciton. The organic EL element is a luminous element utilizing the release (fluorescent light or phosphorescent light) of light from these excitons, and it is a technique expected as a flat display or lighting of the next generation, but there is still a problem such as the luminous efficiency or durability and manufacturing yield particularly in a large-sized display.

The performance of the organic EL element greatly changes depending on the material contained in each layer of the element, and thus the creation of a new material is expected.

Hitherto, an alkali metal halide or the like that is unstable to moisture or the like has been used as the electron injecting material for the organic EL element, and an alternative is required from the viewpoint of lifespan and production stability.

In recent years, a compound that is called an electride and has a significantly shallow work function while being a stable inorganic substance has been developed and attracted attention as a doping material to shallow the work function of a transparent electrode (for example, see Patent Literatures 1 to 3). Furthermore, it has recently become possible to manufacture an amorphous 12CaO.7Al₂O₃ electride (hereinafter, also referred to as C12A7). This is expected to be utilized as the electron injection layer of an organic EL element as it has been revealed that this can be formed into a film by sputtering (for example, see Non Patent Literatures 1 to 3).

Meanwhile, in the organic light emitting diode (OLED) display, the thin film transistor (TFT) portion to drive the pixel has being changed from polysilicon of a p-type semiconductor of the prior art to an oxide semiconductor such as IGZO (Indium Gallium Zinc Oxide) of an n-type semiconductor.

In addition, it is known that the polarity of the diode to be connected to the TFT using an n-type semiconductor material is the cathode to be advantageous upon designing the circuit. The TFT that can cope with a characteristic change of the organic EL element over time differs depending on the limitation on the relation of the counter electrode (common electrode) of the organic EL element with the polarity of TFT (for example, see Patent Literature 4).

In other words, in the case of desiring to obtain a stable display without having a variation between pixels over time, it is known that a cathode common (sequentially layered) type organic EL element is desirable in the case of a TFT using a p-type semiconductor of the prior art, but an anode common (reversely layered) type organic EL element is preferable in the case of a TFT using an n-type semiconductor.

One of the problems that are likely to be caused in the case of fabricating a reversely layered type organic EL element is the flatness of ITO that is the lower electrode. In general, ITO (Indium Tin Oxide) has a relatively great surface roughness, and thus dark spots are caused by the occurrence of leakage or the like when this is not well flattened, and the lifespan of the element is shortened.

It is preferable to form a relatively thick (to 10 nm) electron injection layer as the layer to be on ITO in order to suppress the occurrence of leakage or the like, but a material which functions in such a thickness and exhibits high electron injection properties has not been known so far.

In recent years, however, it has been reported that the electride described above functions even in a thickness of 10 nm to be 10 times thicker as compared to an alkali metal halide known in the prior art, and thus the unevenness of ITO can be well flattened, for example, when being used in the electron injection layer of the reversely layered organic EL element (for example, see Non Patent Literature 3). Hence, the electride is believed to be a promising electron injecting material of a reversely layered type organic EL element that is supposed to be connected to an n-type TFT.

In addition, a top emission type organic EL element in which the aperture ratio at the TFT portion does not decrease has also been developed along with the high definition of the display, and an electride is expected to be a useful material for these as well.

This is because the use of an electride makes it possible to form an electron injection layer having a layer thickness of about 10 nm as described above and thus it is possible to lengthen the distance between the luminous layer and the counter electrode composed of a metal of the organic EL element. In addition, this is because it is possible to decrease the plasmon loss that is an obstacle to the improvement in light extraction efficiency of the organic EL element and thus it is possible to expect the improvement in lifespan from its chemical stability.

However, the driving voltage of these elements using an electride in the electron injection layer is still higher as compared to a general organic EL element having a sequentially layered constitution, which is a problem to be improved. In addition, the lifespan characteristics (stability) of these organic EL elements using an electride have not been clear.

### CITATION LIST

### Patent Literatures

Patent Literature 1: JP 2013-40088 A
Patent Literature 2: JP 2003-238149 A
Patent Literature 3: JP 2009-193962 A
Patent Literature 4: JP 2003-295792 A

### Non Patent Literatures

Non Patent Literature 1: F. J. Tehan, B. L. Barrett, J. L. Dye, J. Am. Chem. Soc., 1974, 96, 7203-7208
Non Patent Literature 2: S. Watanabe et al., 19th International Display Workshops (IDW/AD'12), Japan, 2012, p 1871-1872
Non Patent Literature 3: T. Watanabe et al., The 13th International Meeting on Information Display, Korea, 2013, p 42-43

### SUMMARY OF INVENTION

### Technical Problem

The present invention has been made in view of the above problem and circumstances, and an object thereof is to provide an organic electroluminescent element which has a high luminous efficiency and an excellent driving voltage and exhibits excellent stability, and a display device and a lighting device that are equipped with the organic electroluminescent element.

### Solution to Problem

The present inventors have investigated on the factor or the like of the above problem in order to achieve the above object, and as a result, it has been found out that the electron mobility or the like is improved and the performance of an organic EL element is improved by meeting the requirements that the electron injection layer included in the organic EL element contains an electride, the electron transport layer contains an organic compound containing a nitrogen atom having a lone pair of electrons that does not participate in aromaticity, and the like, whereby the present invention has been completed.

In other words, the object according to the present invention is achieved by the following means.

1. An organic electroluminescent element including at least an electron injection layer, an electron transport layer, and a luminous layer between a positive electrode and a negative electrode, wherein

the electron injection layer contains an electride,

the electron transport layer contains an organic compound having a nitrogen atom,

at least one of the nitrogen atoms has a lone pair of electrons that does not participate in aromaticity, and

the lone pair of electrons does not coordinate a metal.

2. The organic electroluminescent element according to Item. 1, wherein the electron injection layer contains at least 12CaO.7Al₂O₃ as the electride.

3. The organic electroluminescent element according to Item. 1 or 2, wherein a content ratio [n/M] of effective lone pair of electrons is in a range of from 4.0×10⁻³ to 2.0×10⁻² when the number of the lone pair of electrons is denoted as the number n of effective lone pair of electrons and a molecular weight of the organic compound is denoted as M.

4. The organic electroluminescent element according to any one of Items. 1 to 3, wherein the organic compound is a low molecular weight compound having a structure represented by the following Formula (1), a polymer compound having a structural unit represented by the following Formula (2), or a polymer compound having a structural unit represented by the following Formula (3):

[Chemical Formula 1]

(A₁)ₙ₁-y₁ Formula (1)

[in Formula (1), A₁ represents a monovalent nitrogen atom-containing group. n1 represents an integer of 2 or more. A plurality of A₁ may be the same as or different from one another. y₁ represents a n1-valent linking group or a single bond.]

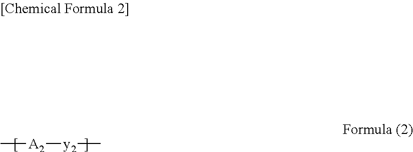

[in Formula (2), A₂ is a divalent nitrogen atom-containing group. y₂ represents a divalent linking group or a single bond.]

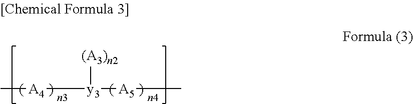

[in Formula (3), A₃ represents a monovalent nitrogen atom-containing group. A₄ and A₅ each independently represent a divalent nitrogen atom-containing group. n2 represents an integer of 1 or more, and n3 and n4 each independently represent an integer of 0 or 1. y₃ represents a (n2+2)-valent linking group.].

5. The organic electroluminescent element according to Item. 4, wherein the organic compound is a low molecular weight compound represented by Formula (1) above.

6. The organic electroluminescent element according to Item. 4 or 5, wherein the organic compound contains a pyridine ring in its chemical structure.

7. The organic electroluminescent element according to any one of Items. 4 to 6, wherein the organic compound has a structure represented by the following Formula (4):

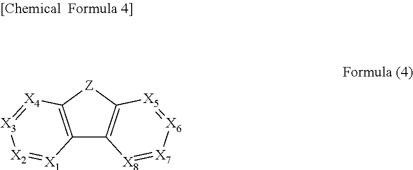

[in Formula (4), Z represents CR₁R₂, NR₃, O, S, PR₄, P(O)R₅, or SiR₆R₇. X₁ to X₈ represent CR₈ or N, and at least one of them represents N. R₁ to R₈ each independently represent a single bond, a hydrogen atom, a substituted or unsubstituted alkyl group having from 1 to 20 carbon atoms, a substituted or unsubstituted cycloalkyl group having from 3 to 20 carbon atoms, a substituted or unsubstituted aryl group having from 6 to 30 carbon atoms, a substituted or unsubstituted heteroaryl group having from 1 to 30 carbon atoms, or a substituted or unsubstituted alkyloxy group having from 1 to 20 carbon atoms.].

8. The organic electroluminescent element according to Item. 7, wherein X₃ or X₄ in Formula (4) above represents a nitrogen atom.

9. The organic electroluminescent element according to any one of Items. 4 to 8, wherein the organic compound has a structure represented by the following Formula (5):

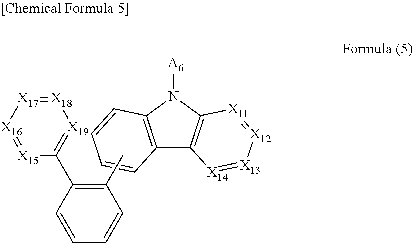

[in Formula (5), A₆ represents a substituent. X₁₁ to X₁₉ each represent C(R₂₁) or N. R₂₁ represents a hydrogen atom or a substituent. Provided that, at least one of X₁₅ to X₁₉ represents N.].

10. The organic electroluminescent element according to any one of Items. 1 to 9, wherein the negative electrode is a transparent electrode, and the organic electroluminescent element includes an electron injection layer, an electron transport layer, a luminous layer, a hole transport layer, and a positive electrode on the negative electrode in this order.

11. The organic electroluminescent element according to any one of Items. 1 to 10, wherein the organic compound contains an electron donating dopant.

12. A display device including the organic electroluminescent element according to any one of Items. 1 to 11.

13. A lighting device including the organic electroluminescent element according to any one of Items. 1 to 11.

### Advantageous Effects of Invention

By the means of the present invention described above, it is possible to provide an organic electroluminescent element which has a high luminous efficiency and an excellent driving voltage and exhibits excellent stability, and a display device and a lighting device which are equipped with the organic electroluminescent element.

The mechanism of exertion or action of the effect of the present invention has not been clear, but it is presumed as follows.

In Non Patent Literature 3, an electride has a high driving voltage although it has a level of from 2.4 to 3.1 eV to be close to the LUMO (lowest unoccupied molecular orbital) level of a material used in the electron transport layer and also has a relatively favorable conductivity of 1.0×10⁻² Scm⁻¹. The reason for that is presumed that there is a problem in the interface or interaction between the electride and the electron transport layer.

It is presumed that the interface between the electron transport layer and the electron injection layer (alkali metal halide) of a sequentially layered constitution of the prior art is embedded in the electron injection layer since the molecule of the electron injection layer is significantly small and the interface is a mixed layer in a certain range. Furthermore, it is presumed that the alkali metal halide or the like is partially cleaved and the bond thereof is exchanged by the energy at the time of deposition to be in a state of a reduced alkali metal and mixed in the electron transport layer and the alkali metal halide or the like is not only at the interface between the electron injection layer and the electron transport layer but is also practically joined in a certain thickness. As a result, it is believed that the electrical joining between the layers is favorable and a great applied voltage is not generated in between the real layers.

On the other hand, an electride has a large structure so that the basic structure has a diameter reaching 4 Å, and thus it is believed that the electride is hardly embedded in the electron transport layer and the frequency of interaction with the electron transporting material is small even if a part thereof is embedded. Particularly, in the case of a reversely layered constitution, a flat electride layer is previously formed, and thus it is supposed that the improvement (decrease in driving voltage) in conductivity is more unlikely to occur in such a way that the electron moves between the electride and the electron transporting material to be doped.

Hence, it is presumed that it is essentially required as an electron transporting material suitable for an electride that it interacts with the surface of an electride and further the part having the interaction has a site to transport a charge (electron density of the LUMO is high). In other words, an electron transporting material which contains an organic compound having a nitrogen atom and in which at least one of the nitrogen atoms has a lone pair of electrons that does not participate in aromaticity and also the lone pair of electrons does not coordinate a metal is used as the material for an electron transport layer.

The use of such an electron transporting material makes it possible to shorten the distance between the electron transporting material and the electride surface as this lone pair of atoms interacts with the metal ion constituting the electride and to lower the energy required for charge transfer. In addition, it has been found out that the electron cloud of the LUMO is often spread in the partial structure which contains a nitrogen atom having these lone pairs of atoms and the function of transporting the electron is high, thus favorable electron transport properties are exhibited and the driving voltage is decreased, and as a result, it is possible to increase the efficiency. In addition, it has also been found out that the structural and morphological changes between the electron injection layer and the electron transport layer hardly occur even at the time of driving by such interaction, and thus the stability over time is excellent.

The present inventors have so far acquired the knowledge on silver which is known for that it exerts a unique interaction and, for example, is significantly easily aggregated in the case of depositing a metal on a compound containing nitrogen having such a lone pair of electrons.

Specifically, it has been found out that it is possible to prevent the aggregation of silver by the interaction in the case of depositing silver on a layer of a compound having a lone pair of electrons in an appropriate density so as to form a transparent conductive film that is transparent and highly conductive (WO 2013/073356 A, WO 2013/099867 A, Japanese Patent Application No. 2012-97977, and the like).

Particularly in Japanese Patent Application No. 2012-97977, it has been found out that the content ratio [n/M] of effective lone pair of electrons is related to the extent of interaction with a metal atom when the number of lone pair of electrons that does not participate in aromaticity and does not coordinate a metal on a nitrogen atom is denoted as the effective lone pair of electrons n and the molecular weight is denoted as M. It is disclosed that the silver thin film can have a significantly favorable surface resistance by the use of a compound which has this parameter in a certain range (compound having a content ratio of effective lone pair of electrons in a range of from 2.0×10⁻³ to 2.0×10⁻² and more preferably in a range of from 3.9×10⁻³ to 2.0×10⁻²) (see FIG. 1).

An electride also contains a metal atom, and thus the combination of these compounds with the electride has been systematically investigated on the hypothesis that a compound having such a lone pair of electrons also favorably interacts with the electride. As a result, a tendency has been successfully found out that the electron injection properties are improved in a case in which the density of the content ratio of effective lone pair of electrons in a certain range by measuring the initial driving voltage of the organic EL element using the electride (see FIG. 2). It is possible to judge the electron transport properties by measuring the driving voltage when a current of 2.5 mA/cm² flows instead of the sheet resistance illustrated in FIG. 1.

As can be seen from FIG. 2, it has been found out that the content ratio of effective lone pair of electrons defined by the number n of the lone pair of electrons/molecular weight M is correlated with the electron injection properties of the organic EL element containing an electride and it is possible to obtain an organic EL element having a favorable driving voltage by adjusting the content ratio of effective lone pair of electrons. In addition, it has been found out that it is possible to improve the stability as well and to obtain a more industrially useful organic EL element.

### BRIEF DESCRIPTION OF DRAWINGS

FIG. 1 is a graph illustrating the relation of the content ratio of effective lone pair of electrons in a layer adjacent to a transparent conductive layer containing silver with the sheet resistance.

FIG. 2 is a graph illustrating the relation of the content ratio of effective lone pair of electrons in an electron injection layer with the initial driving voltage.

FIG. 3 is a schematic cross-sectional diagram illustrating an example of an organic EL element of the present invention.

FIG. 4 is a schematic cross-sectional diagram illustrating an example of an organic EL element of the present invention.

FIG. 5 is a schematic cross-sectional diagram illustrating an example of an organic EL element of the present invention.

FIG. 6 is a schematic cross-sectional diagram illustrating an example of an organic EL element of the present invention.

FIG. 7 is an outline diagram of a lighting device.

FIG. 8 is a schematic diagram of a lighting device.

### DESCRIPTION OF EMBODIMENTS

The organic electroluminescent element of the present invention is characterized in that the electron injection layer contains an electride, the electron transport layer contains an organic compound having a nitrogen atom, at least one of the nitrogen atoms has a lone pair of electrons that does not participate in aromaticity, and the lone pair of electrons does not coordinate a metal. This characteristic is a technical feature that is common to the invention according to the claims of claim 1 to claim 13.

Here, the term “electride” is an ionic compound based on the concept that is first proposed by J. L. Dye et al., and it refers to a substance in which the position that is supposed to be occupied by an anion is occupied by an electron (see Non Patent Literature 1).

It has been known that an electride exhibit unique properties since an electron is the same as an anion in terms of having a negative charge but different from an anion in terms of having a small mass and behaving in a quantum mechanical manner.

As an embodiment of the present invention, it is preferable that the electron injection layer contains at least 12CaO.7Al₂O₃ as the electride.

This is because those containing C12A7 can form an electron injection layer which exhibits higher amorphous properties that are useful in an organic EL element, for example, hardly causes a pinhole and a dark spot although 12CaO.7Al₂O₃, 12SrO.7Al₂O₃ (hereinafter, also referred to as S12A7.), and a mixture thereof (12(CaₓSr₁₋ₓ)O.7Al₂O₃ (0<x<1)) are particularly known as an electride.

In addition, it is preferable that the content ratio [n/M] of effective lone pair of electrons is in a range of from 4.0×10⁻³ to 2.0×10⁻² when the number of the lone pair of electrons is denoted as the number n of effective lone pair of electrons and the molecular weight of the organic compound is denoted as M.

This is because it is possible to obtain an organic EL element having a low driving voltage when using an electron transporting material which falls in this range. A compound in this range is presumed to be a preferred electron transporting material as the interaction thereof with a metal ion forming the electride is significantly strong.

The content ratio of effective lone pair of electrons is more preferably in a range of from 5.0×10⁻³ to 1.0×10⁻² and even more preferably in a range of from 5.0×10⁻³ to 7.0×10⁻³.

In addition, it is preferable that the organic compound is a low molecular weight compound having a structure represented by Formula (1) above, a polymer compound having a structural unit represented by Formula (2) above, or a polymer compound having a structural unit represented by Formula (3) above.

This is because it is presumed that the organic compound has a structure in which the nitrogen atom having a lone pair of electrons is present in the shell of the molecule and thus the interaction thereof with an electride of an electron-rich electron injection layer is strengthened as compared to the case of a structure in which the nitrogen atom having a lone pair of electrons is present at the center of the molecule.

In addition, it is preferable that the organic compound is a low molecular weight compound represented by Formula (1) above.

The reason for this is the same as the reason described above, and this is because it is presumed that the interaction thereof with an electride is greater in a molecular structure in which the nitrogen atom having a lone pair of electrons to interact is present in a radial pattern than in a molecular structure in which the nitrogen atom is present in a linear pattern.

In addition, it is preferable that the organic compound contains a pyridine ring in the chemical structure thereof. For example, as a nitrogen-containing group which has a lone pair of electrons, a cyclic group such as a dimethylamino group or a piperidyl group is preferable. Alternatively, a non-cyclic amine compound is also preferable since an arylamine structure does not actually have a coordination force to a metal ion as the lone pair of electrons is used in resonance with an aromatic ring. In addition, examples thereof may include a nitrogen-containing heteroaromatic ring which has a nitrogen atom at a position exhibiting double bond properties such as a pyridyl group and an oxazole group and a cyano group.

Among these various nitrogen atom-containing groups, a pyridyl group has a strong coordination force and a structure on the plane, and thus it is presumed that it is likely to obtain an electron transporting material having a high electron mobility and it is advantageous to electron transport properties after receiving an electron from an electride, and as a result, the driving voltage can be more lowered, a compound having a substituted and condensed or unsubstituted pyridyl group is preferable as A₁ to A₅.

In addition, it is preferable that the organic compound has a structure represented by Formula (4) above.

This is because it is likely to obtain an organic EL element having a high electron mobility and a low driving voltage particularly by the use of such a tricyclic condensed ring structure.

In addition, it is preferable that X₃ or X₄ in Formula (4) above represents a nitrogen atom.

This is because a compound having the position of a nitrogen atom at X₃ or X₄ is believed to have a high coordination force to an electride. The interaction of X₃ or X₄ present apart from Z with an electride is not inhibited by steric hindrance, and the driving voltage can be lowered.

In addition, it is preferable that the organic compound has a structure represented by Formula (5) above.

This is because a structure as represented by Formula (5) above has a relatively high rotational degree of freedom so as to take a steric structure which flexibly interacts with an electride surface. In addition, this is because it is likely to obtain a thin film exhibiting high amorphous properties, the mobility is hardly lowered, and it is useful for both the efficiency and lifespan of an organic EL element when the organic compound has the structure represented by Formula (5).

In addition, it is preferable that the negative electrode is a transparent electrode, and the organic electroluminescent element has an electron injection layer, an electron transport layer, a luminous layer, a hole transport layer, and a positive electrode on the negative electrode in this order, namely a reversely layered constitution.

This is because it is required to form an electride layer on the organic layer (electron transport layer) by sputtering and the electron transport layer is possibly damaged by sputtering in the case of a sequentially layered constitution.

In addition, it is preferable that the organic compound contains an electron donating dopant.

This is because it is possible to increase the conductivity of the electron transport layer and to obtain an electron transport layer having a thicker layer thickness when an electron donating dopant is contained.

This is because, in the same manner as in the electron injection layer, a decrease in plasmon loss is led when a thick electron transport layer can be formed and thus the light extraction efficiency is improved, and further it is possible to use a cavity effect of improving the color purity as the optical interference is adjusted by changing the layer thickness of the electron transport layer in a display element, and thus it is possible to obtain a luminous color having a higher color purity.

As described above, the organic electroluminescent element of the present invention can take the process window (available range of the layer thickness of the electron transport layer) for improving the color purity of the luminous color in a wide range so as to be suitably equipped in a display device. This makes it possible to improve the luminous efficiency, the driving voltage, and the stability.

In addition, the organic electroluminescent element of the present invention can decrease the plasmon loss so as to be suitably equipped in a lighting device. This makes it possible to improve the luminous efficiency, the driving voltage, and the stability.

Hereinafter, the present invention and the constituents thereof and modes and aspects for carrying out the present invention will be described in detail. Incidentally, in the present application, the term “to” is used in the meaning to include the numerical values described before and after the term as the lower limit value and the upper limit value, respectively.

<<Constitutional Layer of Organic EL Element>>

The organic EL element of the present invention is an organic EL element which has at least an electron injection layer, an electron transport layer, and a luminous layer between a positive electrode and a negative electrode and in which the electron injection layer contains an electride, the electron transport layer contains an organic compound having a nitrogen atom, at least one of the nitrogen atoms has a lone pair of electrons that does not participate in aromaticity, and the lone pair of electrons does not coordinate a metal.

Examples of the representative element constitution of the organic EL element of the present invention may include the following constitutions, but it is not limited thereto.

(1) Negative electrode/electron injection layer/electron transport layer/luminous layer/hole transport layer/positive electrode

(2) Negative electrode/electron injection layer/electron transport layer/luminous layer/hole transport layer/hole injection layer/positive electrode

(3) Negative electrode/electron injection layer/electron transport layer/hole blocking layer/luminous layer/hole transport layer/hole injection layer/positive electrode

(4) Negative electrode/electron injection layer/electron transport layer/luminous layer/electron blocking layer/hole transport layer/hole injection layer/positive electrode

(5) Negative electrode/electron injection layer/electron transport layer/hole blocking layer/luminous layer/electron blocking layer/hole transport layer/hole injection layer/positive electrode

In other words, it is preferable that the negative electrode is a transparent electrode in the organic EL element of the present invention and the organic EL element has an electron injection layer, an electron transport layer, a luminous layer, a hole transport layer, and a positive electrode on the negative electrode in this order.

The constitutions of layers described above are the so-called reversely layered constitution of layers, but it is also possible to preferably use the sequentially layered constitutions to be described below.

(6) Positive electrode/hole transport layer/luminous layer/electron transport layer/electron injection layer/negative electrode

(7) Positive electrode/hole injection layer/hole transport layer/luminous layer/electron transport layer/electron injection layer/negative electrode

(8) Positive electrode/hole injection layer/hole transport layer/(electron blocking layer/)luminous layer/(hole blocking layer/) electron transport layer/electron injection layer/negative electrode

The luminous layer according to the present invention is constituted by a single layer or plural layers, and a nonluminescent intermediate layer may be provided between the respective luminous layers in a case in which the luminous layer is constituted by plural layers.

As described above, if necessary, a hole blocking layer (also referred to as the hole barrier layer) or an electron injection layer (also referred to as the negative electrode buffer layer) may be provided between the luminous layer and the negative electrode and an electron blocking layer (also referred to as the electron barrier layer) or a hole injection layer (also referred to as positive electrode buffer layer) may be provided between the luminous layer and the positive electrode.

The electron transport layer according to the present invention is a layer which has a function of transporting an electron, and the electron injection layer and the hole blocking layer are also included in the electron transport layer in a broad sense. In addition, it may be constituted by plural layers.

The hole transport layer used in the present invention is a layer which has a function of transporting a hole, and the hole injection layer and the electron blocking layer are also included in the hole transport layer in a broad sense. In addition, it may be constituted by plural layers.

In the representative constitutions of the element described above, the layers other than the positive electrode and the negative electrode are also referred to as the organic layer or the organic functional layer, but they can also contain an inorganic substance.

(Tandem Structure)

In addition, the organic EL element of the present invention may be an element having a so-called tandem structure in which a plurality of luminous units including at least an electron injection layer, an electron transport layer, and a luminous layer are stacked.

Examples of the representative constitution of the element having a tandem structure may include the following constitution.

Positive electrode/first luminous unit/intermediate layer/second luminous unit/intermediate layer/third luminous unit/negative electrode

Here, the first luminous unit, the second luminous unit and the third luminous unit may all be the same as or different from one another. In addition, two luminous units may be the same and the other may be different therefrom.

The plurality of luminous units may be stacked directly or via an intermediate layer, and the intermediate layer is also generally called the intermediate electrode, the intermediate conductive layer, the charge generating layer, the electron withdrawing layer, the connecting layer, or the intermediate insulating layer, and it is possible to use a known constitution of material as long as it is a layer which has a function of supplying an electron to a layer adjacent to the positive electrode side and a hole to a layer adjacent to the negative electrode side, respectively.

Examples of the material used in the intermediate layer may include a conductive inorganic compound layer of ITO, IZO (indium zinc oxide), ZnO₂, TiN, ZrN, HfN, TiOx, VOx, CuI, InN, GaN, CuAlO₂, CuGaO₂, SrCu₂O₂, LaB₆, RuO₂, or Al, a bilayer film of Au/Bi₂O₃, a multi-layer film of SnO₂/Ag/SnO₂, ZnO/Ag/ZnO, Bi₂O₃/Au/Bi₂O₃, TiO₂/TiN/TiO₂, or TiO₂/ZrN/TiO₂, a conductive organic substance layer of a fullerene such as C₆₀ or oligothiophene, and a conductive organic compound layer of a metal phthalocyanine, a metal-free phthalocyanine, a metal porphyrin, or a metal-free porphyrin, but the present invention is not limited thereto.

Examples of the preferred constitution of the luminous unit may include those obtained by excluding the positive electrode and the negative electrode from the constitutions of (1) to (8) exemplified as the representative constitution of the element, but the present invention is not limited thereto.

Specific examples of the tandem type organic EL element may include the constitution of element or the constitutional material described in U.S. Pat. No. 6,337,492, U.S. Pat. No. 7,420,203, U.S. Pat. No. 7,473,923, U.S. Pat. No. 6,872,472, U.S. Pat. No. 6,107,734, U.S. Pat. No. 6,337,492, WO 2005/009087 A, JP 2006-228712 A, JP 2006-24791 A, JP 2006-49393 A, JP 2006-49394 A, JP 2006-49396 A, JP 2011-96679 A, JP 2005-340187 A, JP 4,711,424 B1, JP 3,496,681 B1, JP 3,884,564 B1, JP 4,213,169 B1, JP 2010-192719 A, JP 2009-076929 A, JP 2008-078414 A, JP 2007-059848 A, JP 2003-272860 A, JP 2003-045676 A, WO 2005/094130 A, and the like, but the present invention is not limited thereto.

Hereinafter, the respective layers which constitute the organic EL element of the present invention will be described.

<<Electron Injection Layer>>

The electron injection layer (also referred to as the “negative electrode buffer layer”) according to the present invention is a layer that is provided between the negative electrode and the luminous layer in order to lower the driving voltage and to improve the luminescent brightness, and it is described in detail in the “Organic EL Element and its Industrialization Front (Nov. 30, 1998 published by (C) NTS, Inc.)”, Part II, Chapter 2 “Electrode Material” (pp. 123-166).

The electron injection layer in the present invention is a layer that is present between the negative electrode and the luminous layer as described above or between the negative electrode and the electron transport layer.

The electron injection layer has been often a significantly thin film and the thickness of the layer (film) has been often in a range of from 0.1 to 3 nm although it also depends on the material in the prior art. However, it is preferable that a thicker electron injection layer can be formed in the case of using a cavity effect for decreasing the plasmon loss or adjusting the color purity as described above and in the case of a reversely layered organic EL element. It is preferable that the electron injection layer can be formed in a layer thickness of from 3 to 20 nm particularly in order to cover the unevenness of ITO. The layer thickness is more preferably from 5 to 15 nm. Only an electride has been currently found out as the electron injection layer to function in such a layer thickness.

Hence, the electron injection layer according to the present invention contains an electride as an essential component. Specific examples thereof may include an electride (C12A7, S12A7, or the like) composed of calcium or strontium as described in Patent Literatures 1 and 2. It is possible to preferably use an electride that is in a crystalline state or in an amorphous state, but it is preferable that the electride is amorphous in consideration of the durability (occurrence of leakage and dark spots, and the like) of the organic EL element.

In addition, it is preferable to use C12A7 (12CaO.7Al₂O₃) as the electride since it is likely to obtain a more amorphous thin film. The details thereon are also described in JP 6-325871 A, JP 9-17574 A, JP 10-74586 A, and JP 2013-40088 A.

Incidentally, although the characteristics (concentration of electrons and work function) of the electride composed of C12A7 change depending on the process in some cases, the concentration of electrons is preferably in a range of from 2.0×10¹⁸ to 2.3×10²¹/cm³ and more preferably in a range of from 2.0×10²⁰ to 2.0×10²¹/cm³. In addition, although the work function is correlated with the concentration of electrons to some extent, the value measured as the work function in a film state (a method generally called ultraviolet photoelectron spectroscopy (UPS) and the like) is preferably from 2.5 to 3.5 eV and more preferably from 2.8 to 3.2 eV.

In addition, the root mean square roughness RMS (measurable using an atomic force microscope (AFM), an intermolecular force microscope, and the like) of the layer containing an electride is preferably in a range of from 0.1 to 3.0 nm and more preferably in a range of from 0.2 to 2.0 nm.

As a specific example of another material that is preferably used in the electron injection layer, a metal represented by strontium or aluminum, an alkali metal compound represented by lithium fluoride, sodium fluoride, or potassium fluoride, an alkaline earth metal compound represented by magnesium fluoride or calcium fluoride, a metal oxide represented by aluminum oxide, and a metal complex represented by lithium 8-hydroxy quinolate (Liq) may be used concurrently.

In addition, it is also possible to concurrently use the electron transporting material to be described later.

In addition, the material used in the electron injection layer described above may be used singly, or plural kinds thereof may be used concurrently.

<<Electron Transport Layer>>

The electron transport layer in the present invention contains a material which has a function of transporting an electron, and it may have a function of delivering the electrons injected from the negative electrode to the luminous layer.

The total layer thickness of the electron transport layer of the present invention is not particularly limited, but it is usually in a range of from 2 nm to 5 μm, more preferably from 2 to 500 nm, and even more preferably from 5 to 200 nm.

In addition, it has been known that the interference between the light that is directly extracted from the luminous layer and the light that is extracted after being reflected by the electrode positioned at the counter electrode to the electrode to extract the light is caused when the light generated in the luminous layer is extracted from the electrode in the organic EL element. It is possible to efficiently utilize this interference effect by appropriately adjusting the total layer thickness of the electron transport layer to be between 100 and 200 nm in a case in which the light is reflected from the negative electrode.

On the other hand, it is preferable that the electron mobility of the electron transport layer is 10⁻⁵ cm²/Vs or more particularly in a case in which the layer thickness is thick since the voltage is likely to increase when the layer thickness of the electron transport layer is increased.

The electron transport layer according to the present invention contains an organic compound having a nitrogen atom, and at least one of the nitrogen atoms has a lone pair of electrons that does not participate in aromaticity, and the lone pair of electrons does not coordinate a metal.

Here, the term “a lone pair of electrons does not coordinate a metal” refers to that the lone pair of electrons does not coordinate a metal in a state in which the organic compound having a nitrogen atom is a raw material before being introduced into the electron transport layer.

Hence, the nitrogen atom having a lone pair of electrons that does not participate in aromaticity is a nitrogen atom having a lone pair of electrons in a state of not being used as a material for an organic EL element, and it refers to a nitrogen atom of which the lone pair of electrons does not directly participate in the aromaticity of an unsaturated cyclic compound as an essential component.

In other words, it refers to a nitrogen atom of which the lone pair of electrons does not directly participate in the delocalized π electron system on the conjugated unsaturated ring structure (aromatic ring) as an essential one for expression of aromaticity in terms of the chemical structural formula.

The effective lone pair of electrons is defined as a lone pair of electrons that does not participate in aromaticity and does not coordinate a metal among the lone pairs of electrons belonging to the nitrogen atom contained in a compound.

The aromaticity herein refers to an unsaturated ring structure in which atoms having a π electron are lined up in a ring shape, and it is an aromaticity according to the so-called “Huckel's rule”, and it is a condition that the number of electrons contained in the π electron system on a ring is “4n+2” (n=0 or a natural number).

The effective lone pair of electrons as described above is selected depending on whether the lone pair of electrons belonging to a nitrogen atom participates in aromaticity or not regardless of whether the nitrogen atom having a lone pair of electrons itself is a heteroatom constituting an aromatic ring or not. For example, a lone pair of electrons that does not participate in aromaticity is counted as one of effective lone pairs of electrons as long as a certain nitrogen atom has the lone pair of electrons even if the nitrogen atom is a heteroatom which constitutes an aromatic ring. In contrast, a lone pair of electrons of a nitrogen atom is not counted as an effective lone pair of electrons as long as all the lone pairs of electrons of the nitrogen atoms participate in aromaticity even in a case in which the nitrogen atom is not a heteroatom which constitutes an aromatic ring.

In addition, it is preferable that the content ratio [n/M] of effective lone pair of electrons is in a range of from 4.0×10⁻³ to 2.0×10⁻² when the number of the lone pair of electrons that does not participate in aromaticity is denoted as the number n of effective lone pair of electrons and the molecular weight of the organic compound is denoted as M.

In other words, in the present invention, the number n of effective lone pair of electrons to the molecular weight M of such a compound is defined as the content ratio [n/M] of effective lone pair of electrons. Moreover, it is preferable that this content ratio [n/M] of effective lone pair of electrons is in a range of from 4.0×10⁻³ to 2.0×10⁻² in an organic compound having the nitrogen atom contained in the electron transport layer.

Incidentally, a compound having a content ratio [n/M] of effective lone pair of electrons of 2.0×10⁻² or less is preferable since the compound is stable so that the purification through sublimation or deposition is easy.

It is preferable that the content ratio [n/M] of effective lone pair of electrons is in a range of from 4.0×10⁻³ to 2.0×10⁻² in an organic compound having the nitrogen atom contained in the electron transport layer, but the organic compound may be constituted by only such a compound or may be constituted by a mixture of such a compound with another compound. Another compound may or may not contain a nitrogen atom, and the content ratio [n/M] of effective lone pair of electrons may not be in a range of from 4.0×10⁻³ to 2.0×10⁻² even in the case of having a nitrogen atom. Preferably the organic compound is constituted by only those which fall in the above range, and more preferably the electron transport layer is constituted by only a compound of a simple substance.

In a case in which the electron transport layer is constituted by a plurality of compounds, it is preferable that a value that is obtained, for example, by determining the molecular weight M of the mixed compound obtained by mixing these compounds based on the mixing ratio of compounds and determining the number n of the sum of effective lone pairs of electrons to the molecular weight M as the average value of the content ratio [n/M] of effective lone pair of electrons is in the predetermined range described above. In other words, it is preferable that the average value of the content ratio [n/M] of effective lone pair of electrons of the entire organic compounds having the nitrogen atom contained in the electron transport layer is in a predetermined range.

It is possible to efficiently transport an electron through the interaction of the metal atom contained in an electride with the effective lone pair of electrons by providing the electron transport layer to be adjacent to the electron injection layer which contains the electride as the organic compound having the nitrogen atom contained in the electron transport layer has a content ratio of effective lone pair of electrons in a range of from 4.0×10⁻³ to 2.0×10⁻².

Incidentally, even in a case in which the compound contained in the electron transport layer is constituted using a plurality of compounds, the content ratio [n/M] of effective lone pair of electrons on the surface of the electron transport layer on the side in contact with the electron injection layer may be in a predetermined range when the mixing ratio (content ratio) of the compound in the layer thickness direction has a different constitution, but it is preferable that the entire electron transport layer is formed by a compound having a content ratio [n/M] of effective lone pair of electrons in a predetermined range.

In addition, an example is presented in which the improvement in performance is attempted by containing an organic compound having a nitrogen atom in the layer to be adjacent to the transparent electrode containing silver.

A layer containing an organic compound which has a content ratio [n/M] of effective lone pair of electrons in a range of about from 2.0×10⁻³ to 2.0×10⁻² was formed on a transparent electrode containing silver so as to be adjacent to the transparent electrode containing silver, the sheet resistance thereof was measured, and the sheet resistance was a low value of 30Ω/□ or less even though the electrode layer using silver that is substantially responsible for the conductivity is an extremely thin film to be from 2 to 30 nm. From this, it has been confirmed that an electrode layer is formed on the layer containing the organic compound in a substantially uniform layer thickness by a monolayer growth type (Frank-van derMerwe: FM type) film growth.

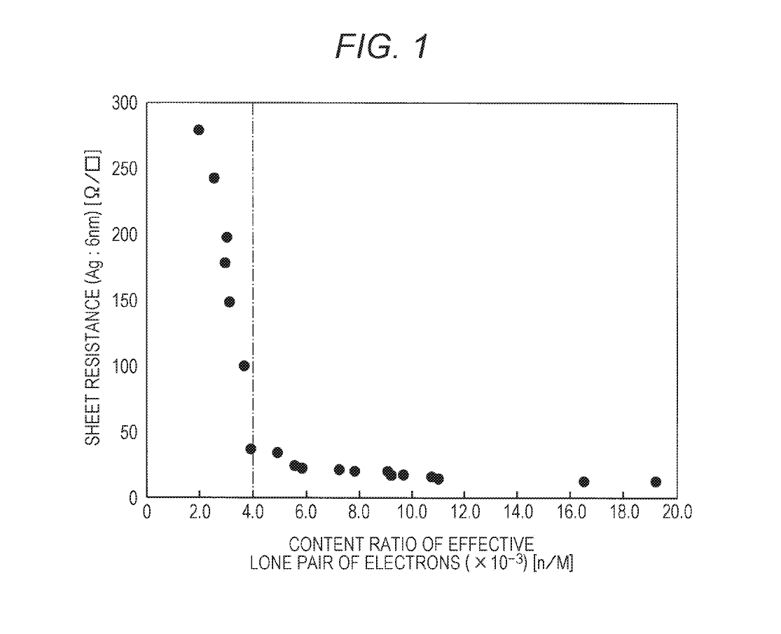

As illustrated in FIG. 1, a graph is illustrated on which the content ratios [n/M] of effective lone pair of electrons of the compounds constituting the layer containing an organic compound and the values of sheet resistance measured for the respective transparent electrode are plotted for a transparent electrodes in which an electrode layer which has a layer thickness of 6 nm and contains silver (Ag) is provided on the upper part of a layer which contains an organic compound using exemplary compounds having the respective values of the content ratios [n/M] of effective lone pair of electrons.

From the graph of FIG. 1, it has been found that the sheet resistance of the transparent electrode tends to be lower as the content ratio [n/M] of effective lone pair of electrons is in a range of about 4.0×10⁻³ or more and particularly the value of the content ratio [n/M] of effective lone pair of electrons is greater. In other words, it has been confirmed that an effect of dramatically lowering the sheet resistance of the transparent electrode is obtained when the content ratio [n/M] of effective lone pair of electrons is in a range of about 4.0×10⁻³ or more. It is believed that this is because such an organic compound and a metal atom form a unique interaction.

[Organic Compound Having Nitrogen Atom]

It is preferable that the organic compound having a nitrogen atom is a low molecular weight compound having a structure represented by the following Formula (1), a polymer compound having a structural unit represented by the following Formula (2), or a polymer compound having a structural unit represented by the following Formula (3).

[Chemical Formula 6]

(A₁)ₙ₁-y₁ Formula (1)

In Formula (1), A₁ represents a monovalent nitrogen atom-containing group. n1 represents an integer of 2 or more. A plurality of A₁ may be the same as or different from one another. y₁ represents an n1-valent linking group or a single bond.

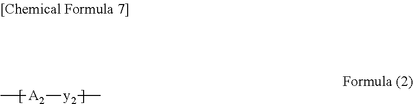

In Formula (2), A₂ is a divalent nitrogen atom-containing group. y₂ represents a divalent linking group or a single bond.

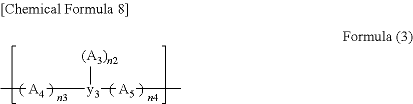

In Formula (3), A₃ represents a monovalent nitrogen atom-containing group. A₄ and A₅ each independently represent a divalent nitrogen atom-containing group. n2 represents an integer of 1 or more, and n3 and n4 each independently represent an integer of 0 or 1. y₃ represents a (n2+2)-valent linking group.

In addition, it is even more preferable that the organic compound is a low molecular weight compound represented by Formula (1) above. This is because it is presumed that the organic compound has a structure in which the nitrogen atom having a lone pair of electrons is present in the shell of the molecule and thus the interaction thereof with an electride of an electron-rich electron injection layer is more strengthened than in the case of a structure in which the nitrogen atom having a lone pair of electrons is at the center of the molecule.

Incidentally, the low molecular weight compound in the present invention means a single molecule which does not have the distribution of the molecular weight of compound. On the other hand, a polymer compound means that an aggregate of a compound which has certain molecular weight distribution and is obtained by reacting a predetermined monomer. However, a compound having a molecular weight of less than 2000 is preferably classified as a low molecular weight compound when the compound is defined by the molecular weight in practical use. The molecular weight of a low molecular weight compound is more preferably 1500 or less and even more preferably 1000 or less. On the other hand, a compound having a molecular weight of 2,000 or more, more preferably 5,000 or more, and even more preferably 10,000 or more is classified as a polymer compound. Incidentally, the molecular weight can be measured by gel permeation chromatography (GPC).

In addition, it is preferable that the organic compound having a nitrogen atom contains a pyridine ring in the chemical structure thereof. This is because it is presumed that it is likely to obtain an electron transporting material having a high electron mobility and it is advantageous to electron transport properties after receiving an electron from the electride, and thus the driving voltage can be more lowered. In addition, a compound having an alkylamino group can shallow the apparent work function by the shift of the vacuum level due to a dipole as described in Adv. Mater., 2011, vol. 23, p 4636 and allows the level of an electride to behave as shallower one. As a result, it is possible to inject an electron to the electron transport layer even at a low voltage.

Incidentally, a layer containing a compound having an amino group and a layer containing a compound having a pyridine ring may be stacked and used. It is possible to synergistically obtain a level shift effect of the work function of the compound having an amino group and a high electron mobility effect of the compound having a pyridine ring in the case of using them in combination.

In addition, it is preferable that the organic compound having a nitrogen atom has a structure represented by the following Formula (4).

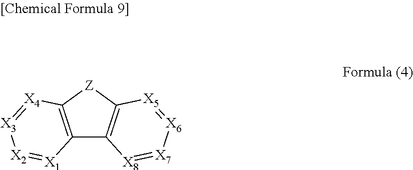

In Formula (4), Z represents CR₁R₂, NR₃, O, S, PR₄, P(O)R₅, or SiR₄R₅. X₁ to X₈ represent CR₆ or N, and at least one of them represents N. R₁ to R₆ each independently represent a single bond, a hydrogen atom, a substituted or unsubstituted alkyl group having from 1 to 20 carbon atoms, a substituted or unsubstituted cycloalkyl group having from 3 to 20 carbon atoms, a substituted or unsubstituted aryl group having from 6 to 30 carbon atoms, a substituted or unsubstituted heteroaryl group having from 1 to 30 carbon atoms, or a substituted or unsubstituted alkyloxy group having from 1 to 20 carbon atoms.

Z represents CR₁R₂, NR₃, O, S, PR₄, P(O)R₅, or SiR₄R₅, but Z is preferably NR₃, O, or S from the viewpoint of obtaining a compound having a high electron mobility. Z is more preferably NR₃ or O and even more preferably NR₃.

In addition, in Formula (4), it is even more preferable that X₃ or X₄ represents a nitrogen atom. This is because a compound having the position of the nitrogen atom at X₃ or X₄ is believed to have a high coordination force to an electride.

In addition, it is preferable that the organic compound having a nitrogen atom has a structure represented by the following Formula (5).

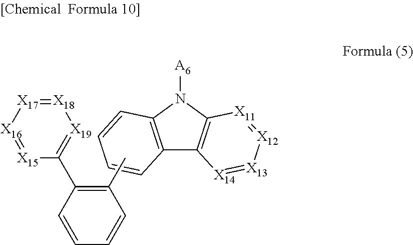

In Formula (5), A₆ represents a substituent. X₁₁ to X₁₉ each represent C(R₂₁) or N. R₂₁ represents a hydrogen atom or a substituent. Provided that, at least one of X₁S to X₁₉ represents N.

Examples of the substituent represented by A₆ may include a substituted or unsubstituted aromatic ring group, a heteroaromatic ring group, an alkyl group, an alkenyl group, an alkynyl group, a cycloalkyl group, a silyl group, a boryl group, and a cyano group. In addition, these substituents may further have a substituent.

[Specific Example of Organic Compound Having Nitrogen Atom]

Hereinafter, specific examples of the organic compound having a nitrogen atom contained in the electron transport layer are presented.

Incidentally, in copper phthalocyanine of Compound ET-146 to be exemplified below, a lone pair of electrons that does not coordinate copper among the lone pairs of electrons belonging to the nitrogen atom is counted as an effective lone pair of electrons. In addition, the polymer compounds (ET-201 to 234) among the exemplified compounds represent a polymer or oligomer having a structure in the parentheses as a repeating structure, and the molecular weight is not particularly limited but those having a molecular weight of 2000 or more are preferable or those having the number of repeating units of 10 or more are preferable. In addition, the molecular weight is preferably less than 1,000,000 since it is preferable to have a solubility in an organic solvent of 0.05% or more in order to coat in the real process. The molecular weight is more preferably less than 100,000 and more preferably less than 50,000. In ET-235, n and m represent the repeating number, respectively, and they may be the same as or different from each other as long as it is a number that satisfies the molecular weight described above.

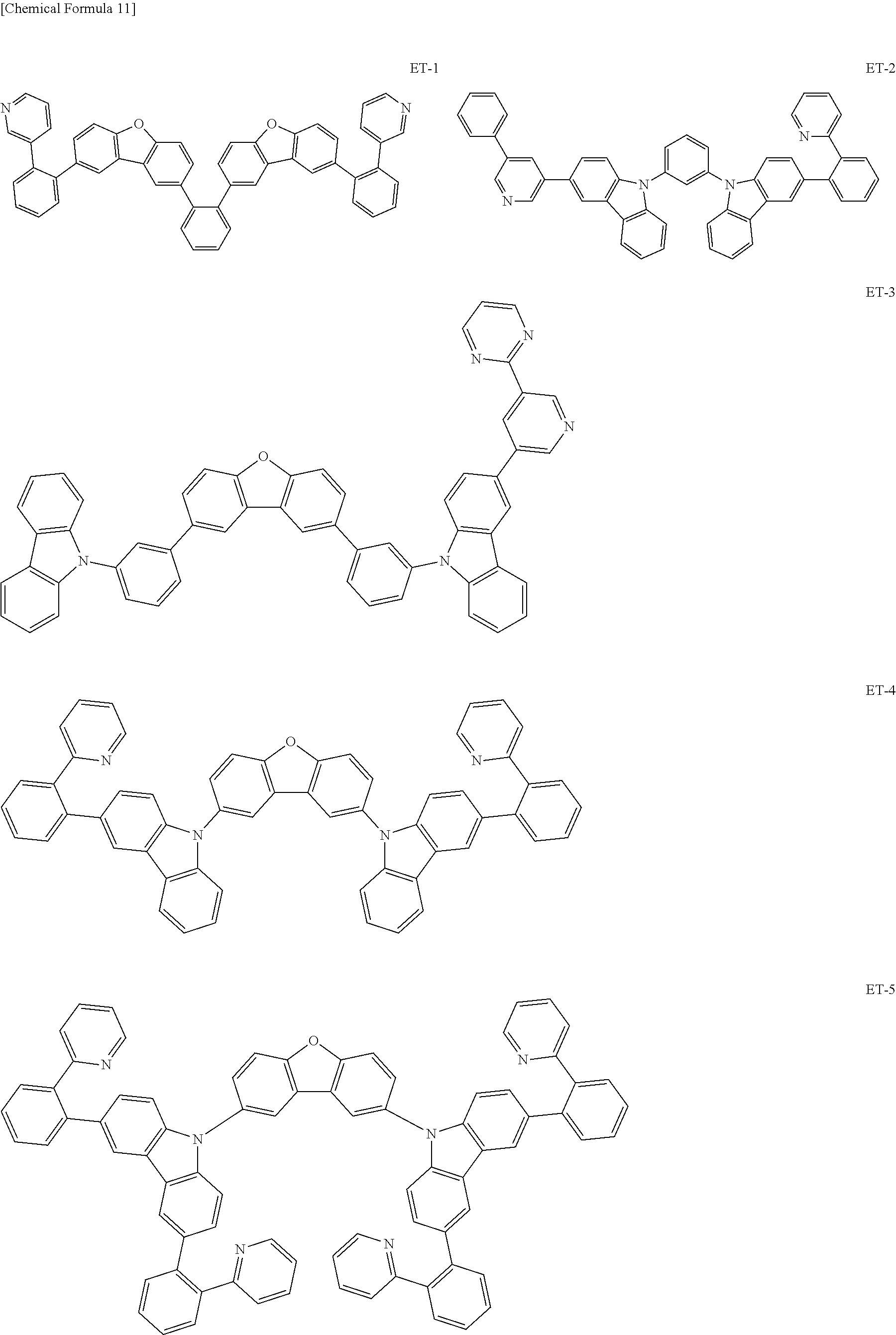

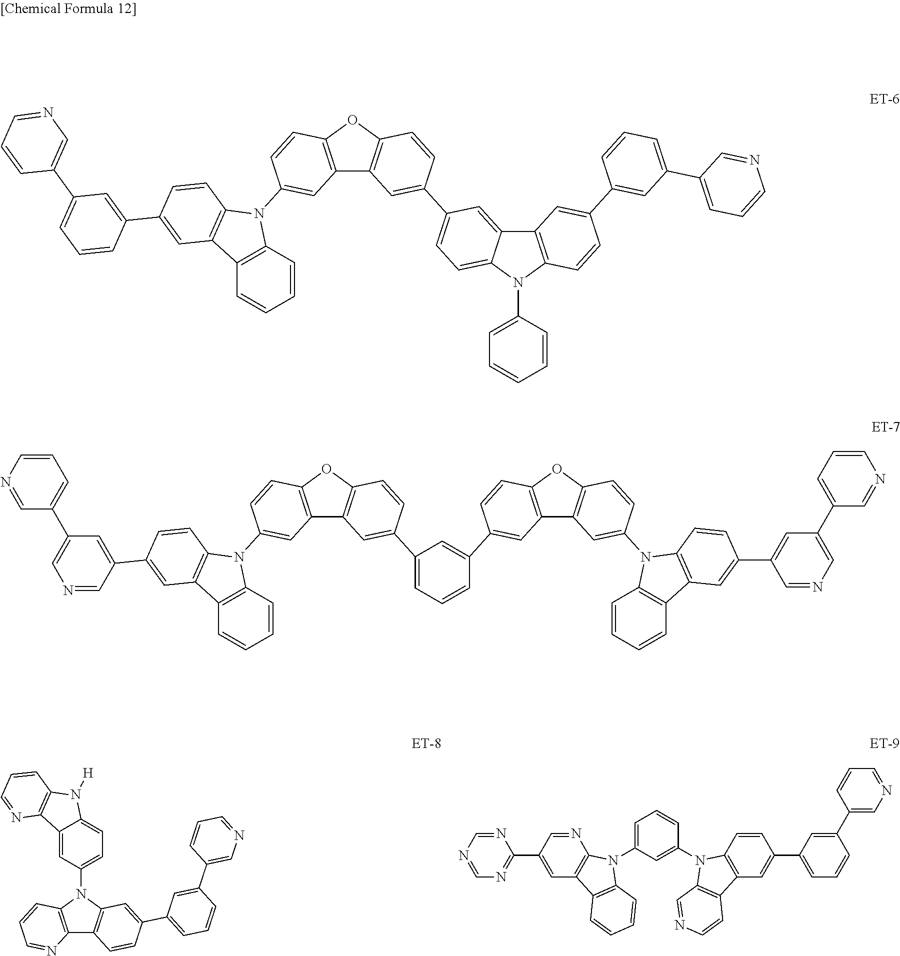 [SMILES: c1ccc(-n2c3ccc(-c4cccc(-c5cccnc5)c4)cc3c3cc(-c4ccc5oc6ccc(-n7c8ccccc8c8cc(-c9cccc(-c%10cccnc%10)c9)ccc87)cc6c5c4)ccc32)cc1 ; c1cncc(-c2cncc(-c3ccc4c(c3)c3ccccc3n4-c3ccc4oc5ccc(-c6cccc(-c7ccc8oc9ccc(-n%10c%11ccccc%11c%11cc(-c%12cncc(-c%13cccnc%13)c%12)ccc%11%10)cc9c8c7)c6)cc5c4c3)c2)c1 ; c1cncc(-c2cccc(-c3ccc4c5ncccc5n(-c5ccc6[nH]c7cccnc7c6c5)c4c3)c2)c1 ; c1cncc(-c2cccc(-c3ccc4c(c3)c3ccncc3n4-c3cccc(-n4c5ccccc5c5cc(-c6ncncn6)cnc54)c3)c2)c1]

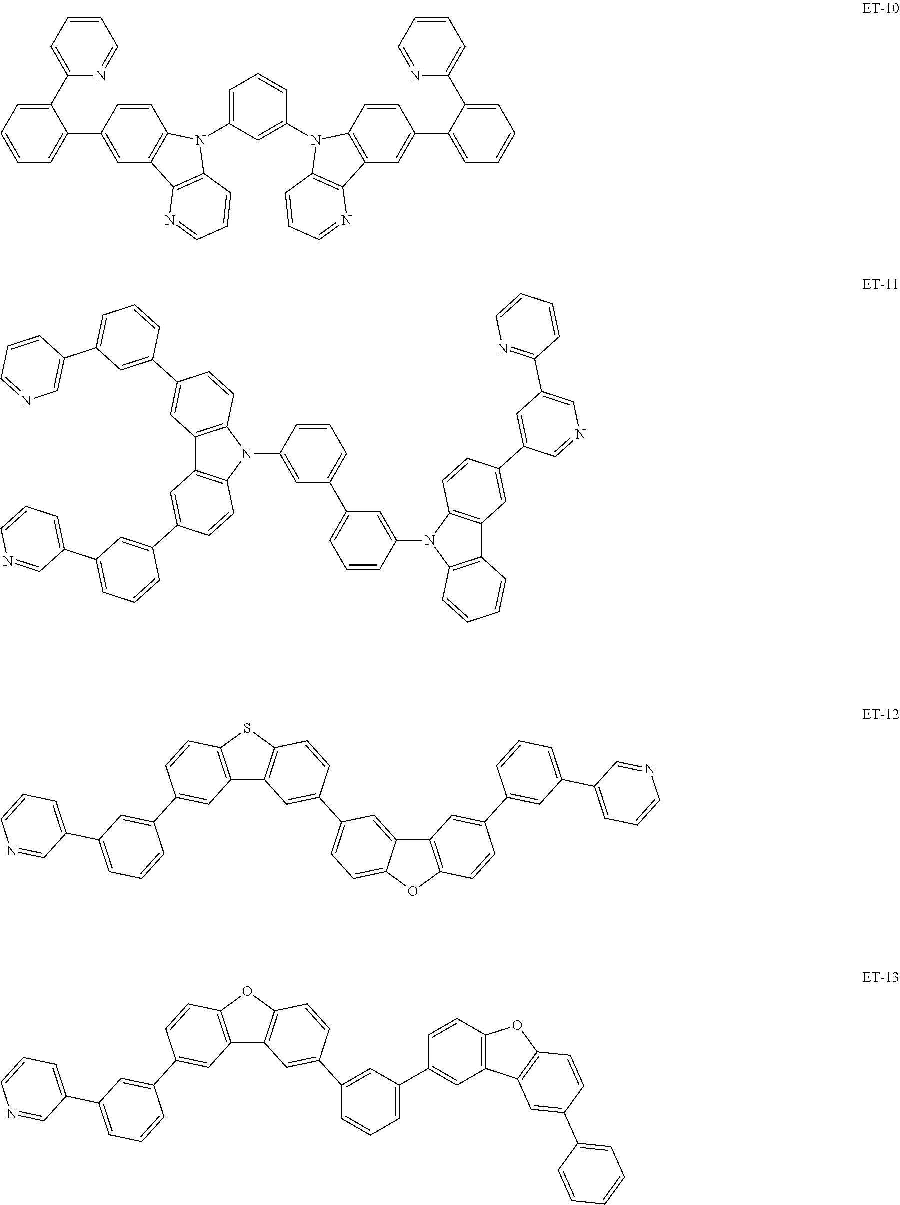 [SMILES: c1ccc(-c2ccccc2-c2ccc3c(c2)c2ncccc2n3-c2cccc(-n3c4ccc(-c5ccccc5-c5ccccn5)cc4c4ncccc43)c2)nc1 ; c1ccc(-c2cncc(-c3ccc4c(c3)c3ccccc3n4-c3cccc(-c4cccc(-n5c6ccc(-c7cccc(-c8cccnc8)c7)cc6c6cc(-c7cccc(-c8cccnc8)c7)ccc65)c4)c3)c2)nc1 ; c1cncc(-c2cccc(-c3ccc4oc5ccc(-c6ccc7sc8ccc(-c9cccc(-c%10cccnc%10)c9)cc8c7c6)cc5c4c3)c2)c1 ; c1ccc(-c2ccc3oc4ccc(-c5cccc(-c6ccc7oc8ccc(-c9cccc(-c%10cccnc%10)c9)cc8c7c6)c5)cc4c3c2)cc1]

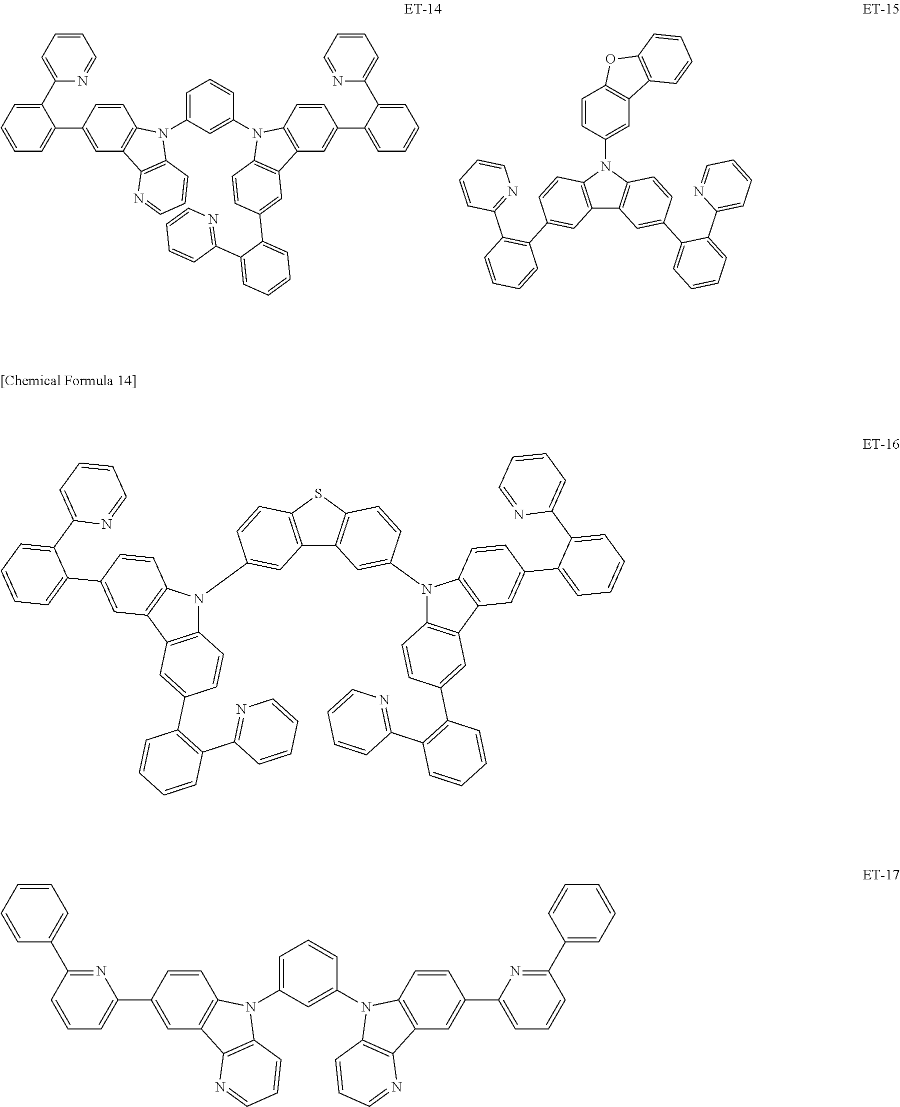 [SMILES: c1ccc(-c2ccccc2-c2ccc3c(c2)c2cc(-c4ccccc4-c4ccccn4)ccc2n3-c2cccc(-n3c4ccc(-c5ccccc5-c5ccccn5)cc4c4ncccc43)c2)nc1 ; c1ccc(-c2ccccc2-c2ccc3c(c2)c2cc(-c4ccccc4-c4ccccn4)ccc2n3-c2ccc3oc4ccccc4c3c2)nc1 ; c1ccc(-c2ccccc2-c2ccc3c(c2)c2cc(-c4ccccc4-c4ccccn4)ccc2n3-c2ccc3sc4ccc(-n5c6ccc(-c7ccccc7-c7ccccn7)cc6c6cc(-c7ccccc7-c7ccccn7)ccc65)cc4c3c2)nc1 ; c1ccc(-c2cccc(-c3ccc4c(c3)c3ncccc3n4-c3cccc(-n4c5ccc(-c6cccc(-c7ccccc7)n6)cc5c5ncccc54)c3)n2)cc1]

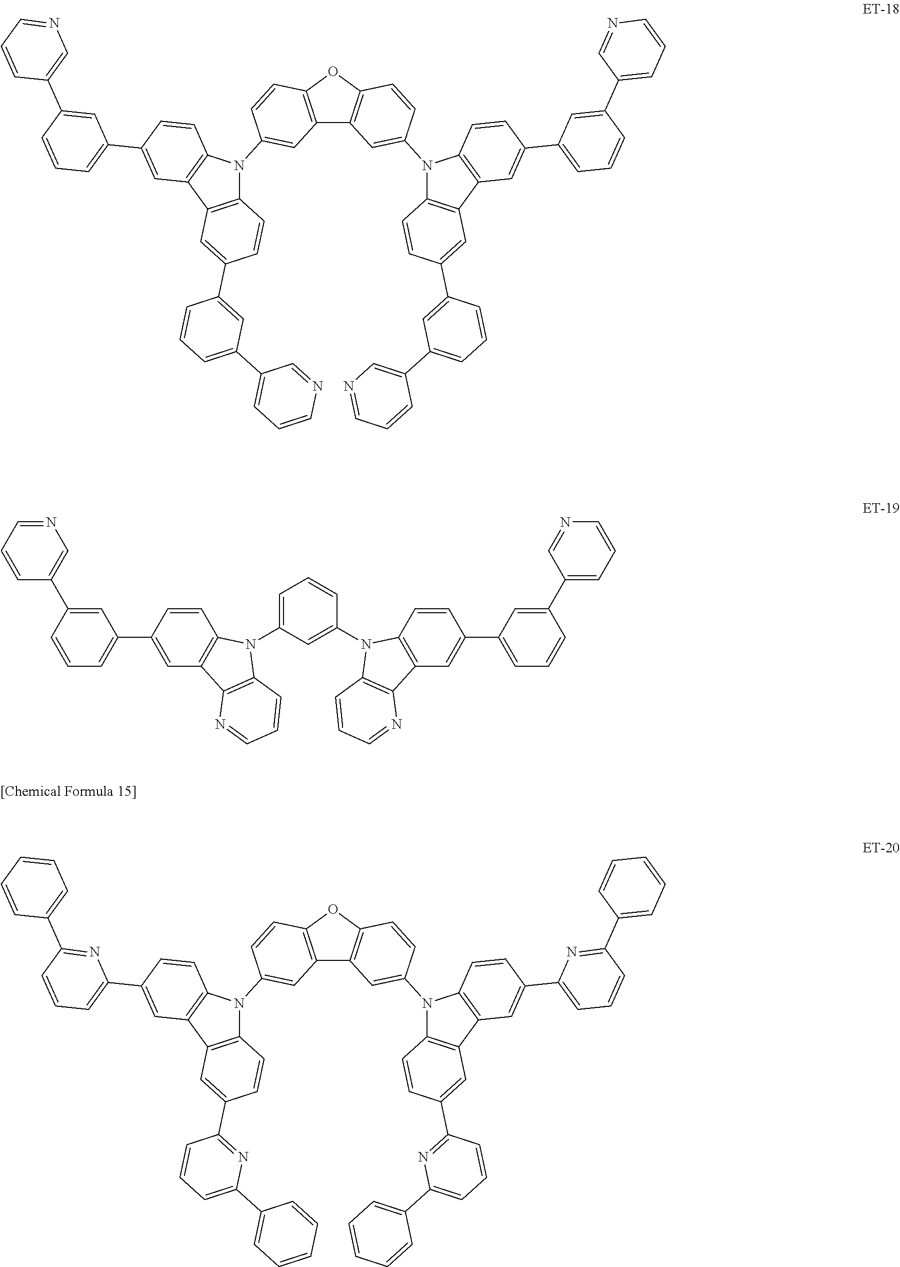 [SMILES: c1cncc(-c2cccc(-c3ccc4c(c3)c3cc(-c5cccc(-c6cccnc6)c5)ccc3n4-c3ccc4oc5ccc(-n6c7ccc(-c8cccc(-c9cccnc9)c8)cc7c7cc(-c8cccc(-c9cccnc9)c8)ccc76)cc5c4c3)c2)c1 ; c1cncc(-c2cccc(-c3ccc4c(c3)c3ncccc3n4-c3cccc(-n4c5ccc(-c6cccc(-c7cccnc7)c6)cc5c5ncccc54)c3)c2)c1 ; c1ccc(-c2cccc(-c3ccc4c(c3)c3cc(-c5cccc(-c6ccccc6)n5)ccc3n4-c3ccc4oc5ccc(-n6c7ccc(-c8cccc(-c9ccccc9)n8)cc7c7cc(-c8cccc(-c9ccccc9)n8)ccc76)cc5c4c3)n2)cc1]

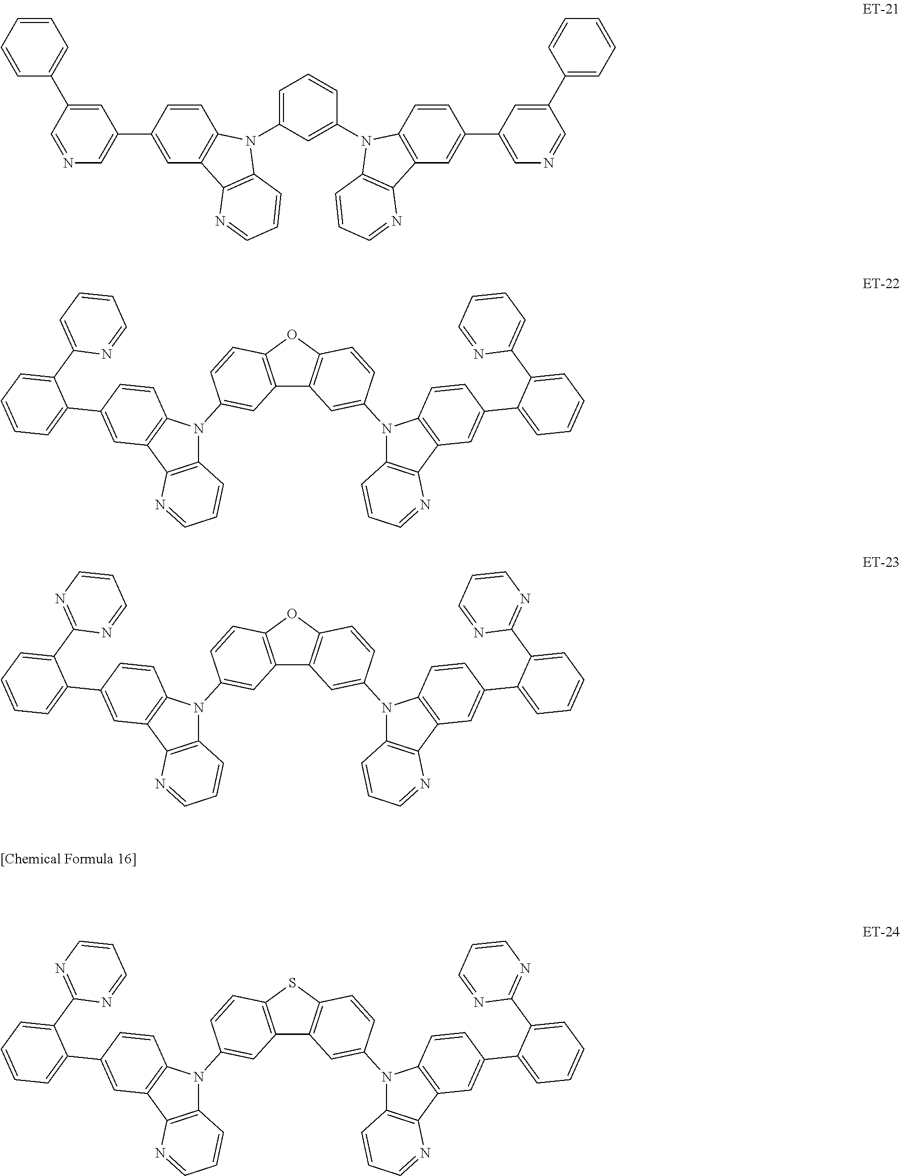 [SMILES: c1ccc(-c2cncc(-c3ccc4c(c3)c3ncccc3n4-c3cccc(-n4c5ccc(-c6cncc(-c7ccccc7)c6)cc5c5ncccc54)c3)c2)cc1 ; c1ccc(-c2ccccc2-c2ccc3c(c2)c2ncccc2n3-c2ccc3oc4ccc(-n5c6ccc(-c7ccccc7-c7ccccn7)cc6c6ncccc65)cc4c3c2)nc1 ; c1cnc(-c2ccccc2-c2ccc3c(c2)c2ncccc2n3-c2ccc3oc4ccc(-n5c6ccc(-c7ccccc7-c7ncccn7)cc6c6ncccc65)cc4c3c2)nc1 ; c1cnc(-c2ccccc2-c2ccc3c(c2)c2ncccc2n3-c2ccc3sc4ccc(-n5c6ccc(-c7ccccc7-c7ncccn7)cc6c6ncccc65)cc4c3c2)nc1]

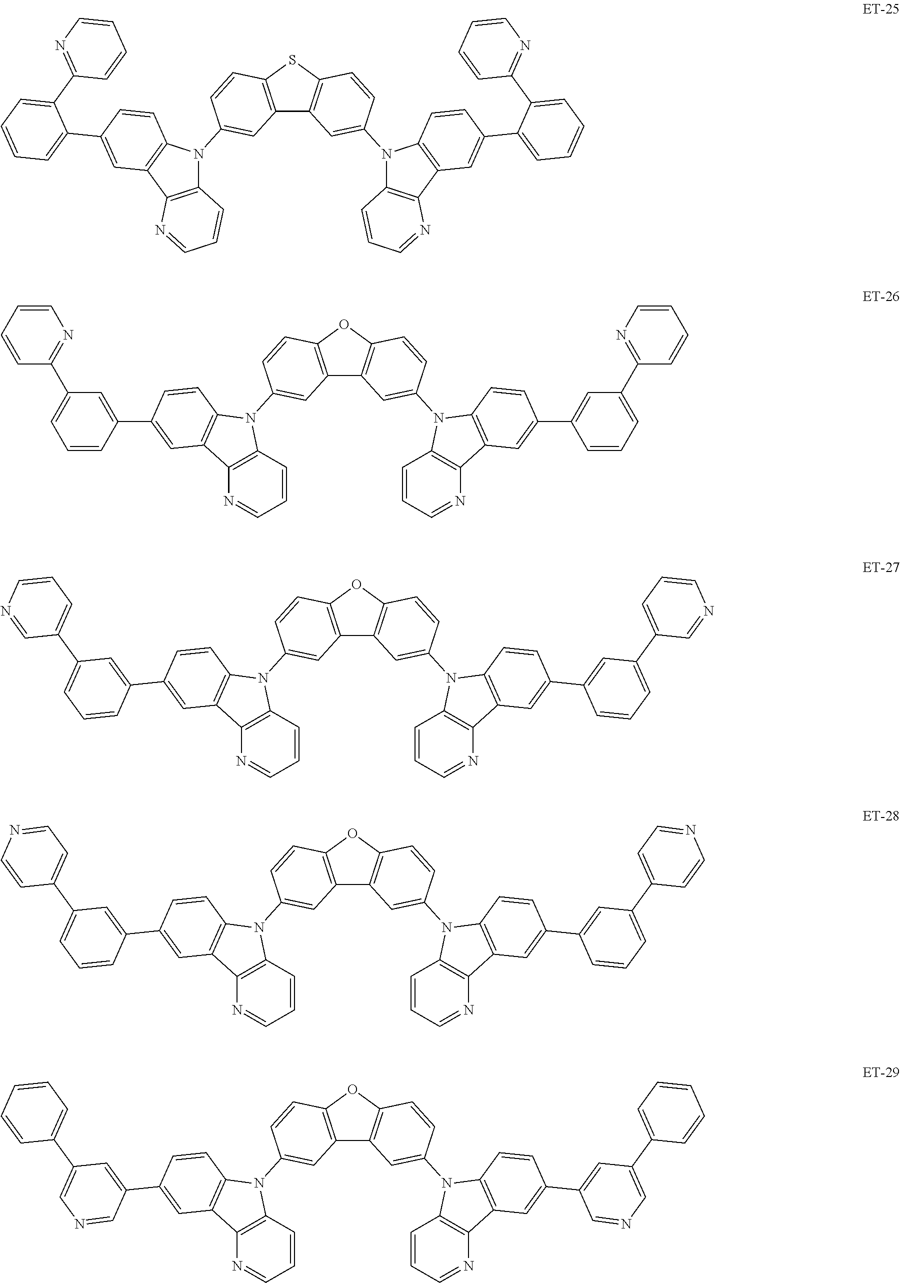

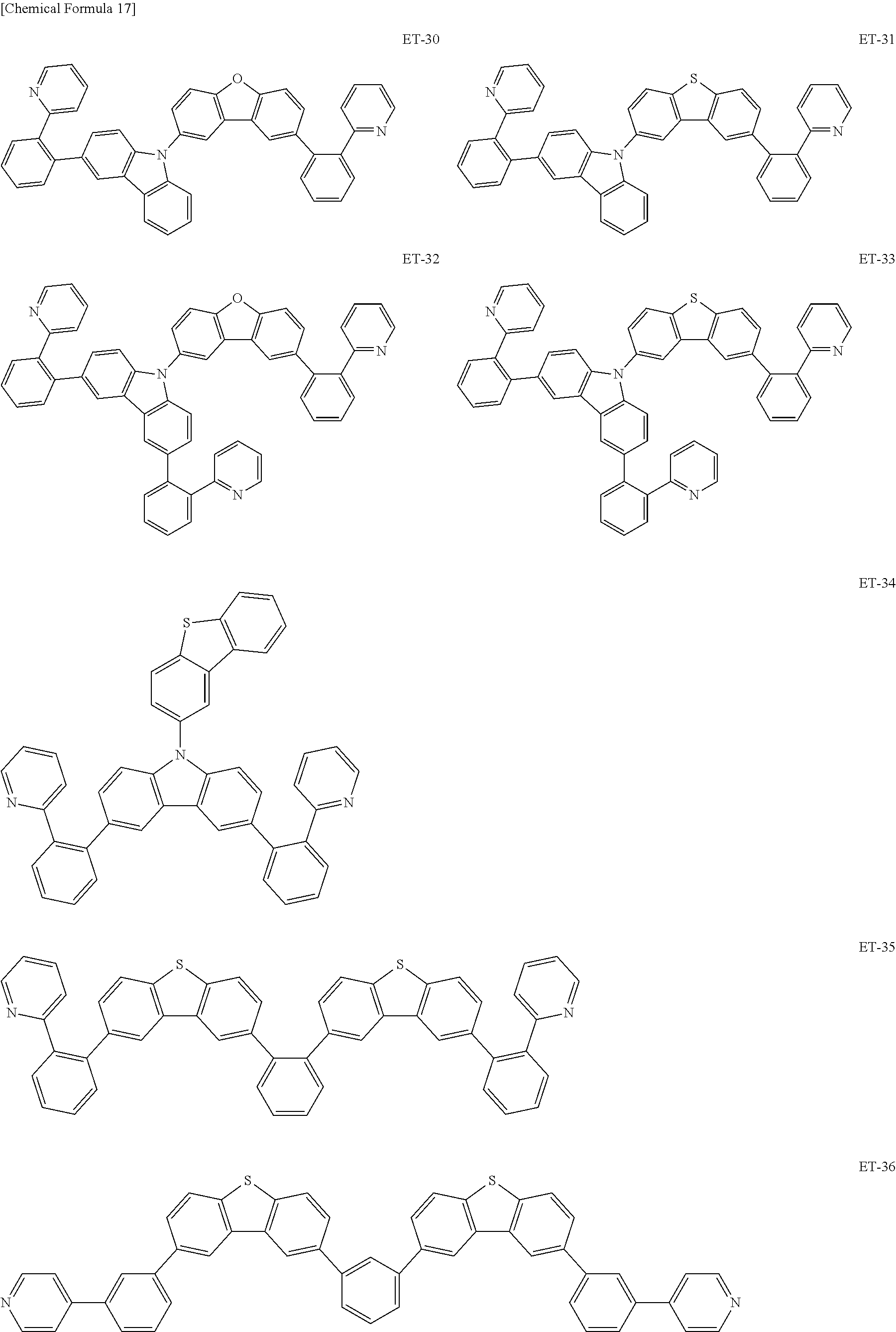

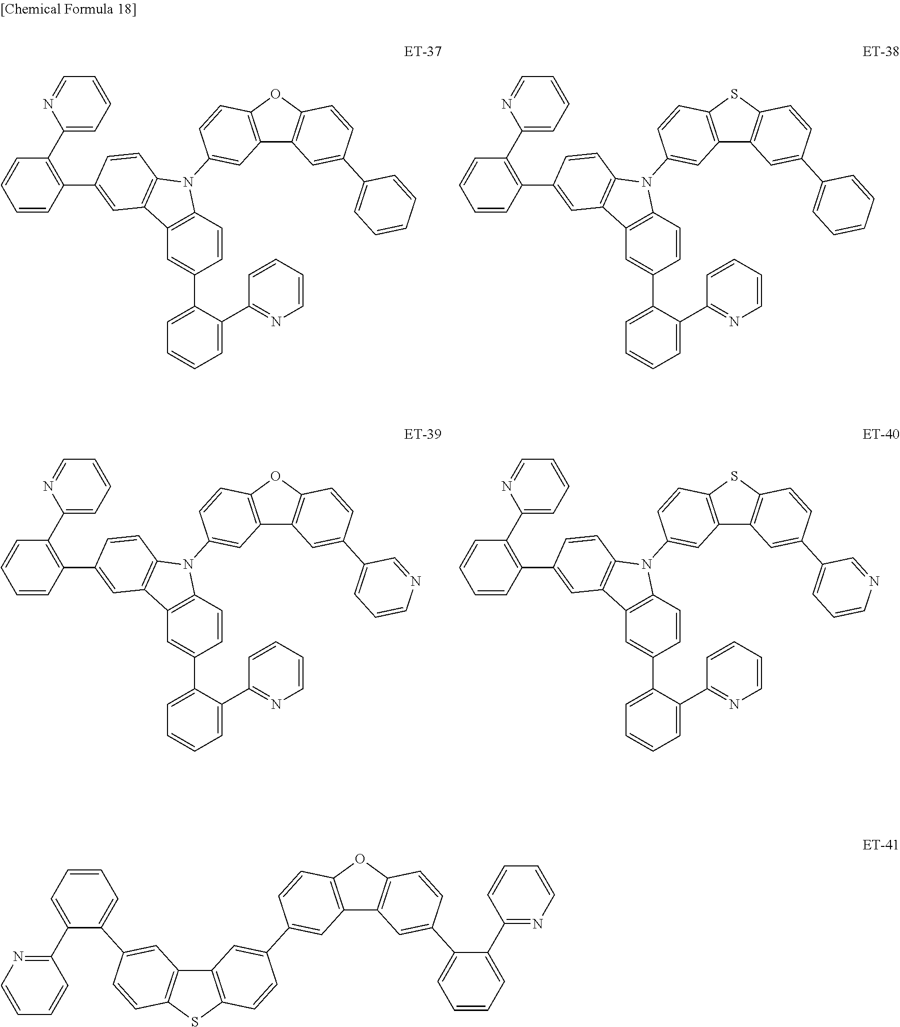 [SMILES: c1ccc(-c2ccc3oc4ccc(-n5c6ccc(-c7ccccc7-c7ccccn7)cc6c6cc(-c7ccccc7-c7ccccn7)ccc65)cc4c3c2)cc1 ; c1ccc(-c2ccc3sc4ccc(-n5c6ccc(-c7ccccc7-c7ccccn7)cc6c6cc(-c7ccccc7-c7ccccn7)ccc65)cc4c3c2)cc1 ; c1ccc(-c2ccccc2-c2ccc3c(c2)c2cc(-c4ccccc4-c4ccccn4)ccc2n3-c2ccc3oc4ccc(-c5cccnc5)cc4c3c2)nc1 ; c1ccc(-c2ccccc2-c2ccc3c(c2)c2cc(-c4ccccc4-c4ccccn4)ccc2n3-c2ccc3sc4ccc(-c5cccnc5)cc4c3c2)nc1 ; c1ccc(-c2ccccc2-c2ccc3oc4ccc(-c5ccc6sc7ccc(-c8ccccc8-c8ccccn8)cc7c6c5)cc4c3c2)nc1]

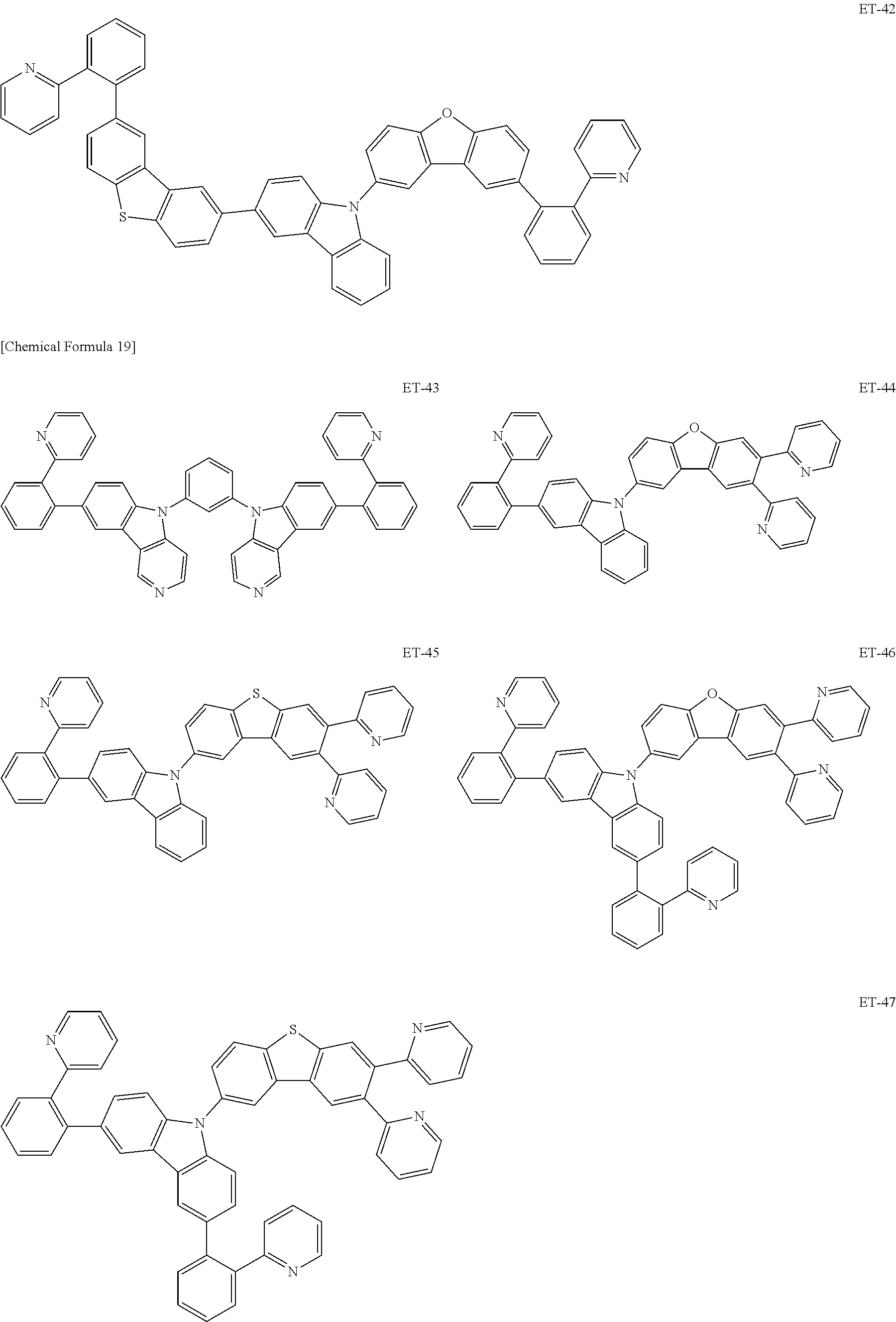

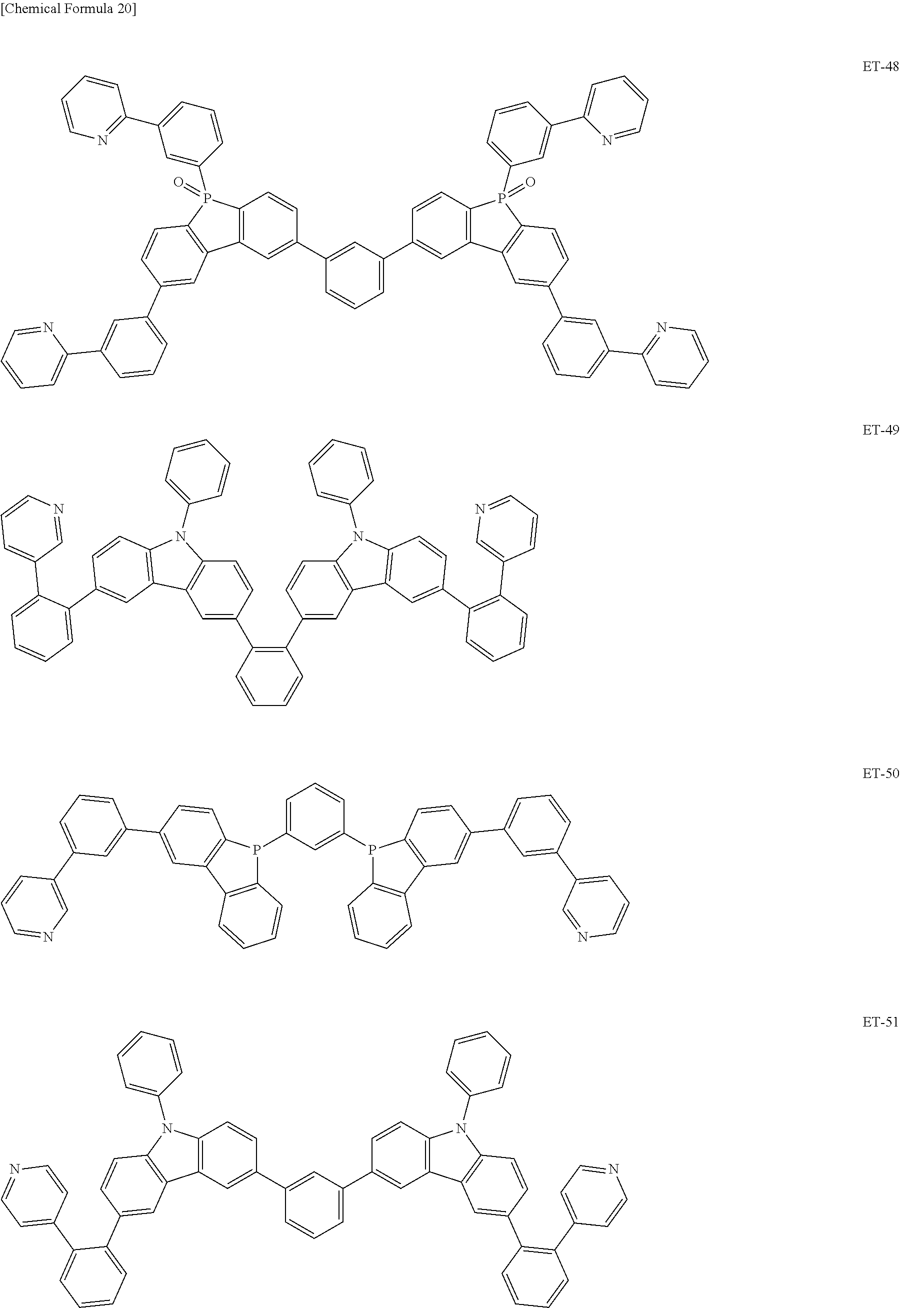 [SMILES: O=P1(c2cccc(-c3ccccn3)c2)c2ccc(-c3cccc(-c4ccc5c(c4)-c4cc(-c6cccc(-c7ccccn7)c6)ccc4P5(=O)c4cccc(-c5ccccn5)c4)c3)cc2-c2cc(-c3cccc(-c4ccccn4)c3)ccc21 ; c1ccc(-n2c3ccc(-c4ccccc4-c4cccnc4)cc3c3cc(-c4ccccc4-c4ccc5c(c4)c4cc(-c6ccccc6-c6cccnc6)ccc4n5-c4ccccc4)ccc32)cc1 ; c1cncc(-c2cccc(-c3ccc4c(c3)c3ccccc3p4-c3cccc(-p4c5ccccc5c5cc(-c6cccc(-c7cccnc7)c6)ccc54)c3)c2)c1 ; c1ccc(-n2c3ccc(-c4cccc(-c5ccc6c(c5)c5cc(-c7ccccc7-c7ccncc7)ccc5n6-c5ccccc5)c4)cc3c3cc(-c4ccccc4-c4ccncc4)ccc32)cc1]

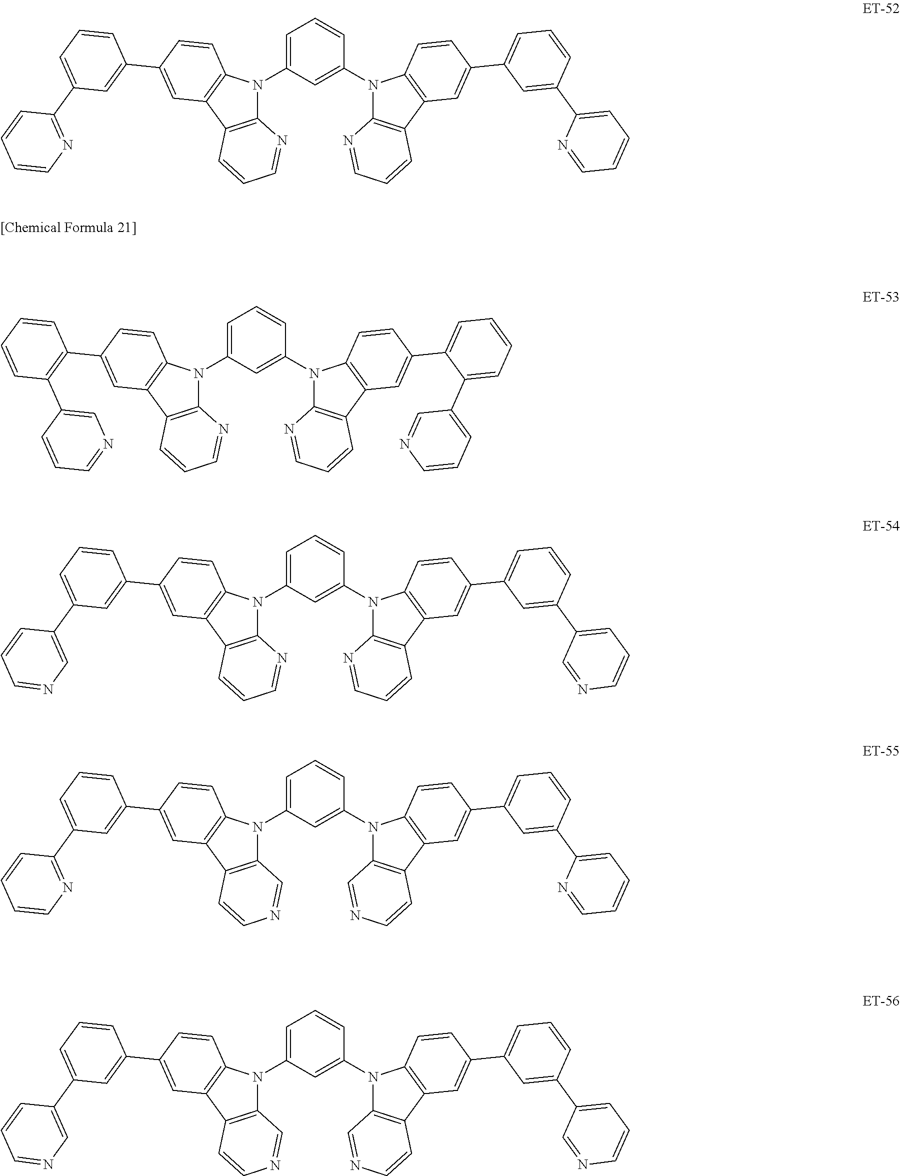 [SMILES: c1ccc(-c2cccc(-c3ccc4c(c3)c3cccnc3n4-c3cccc(-n4c5ccc(-c6cccc(-c7ccccn7)c6)cc5c5cccnc54)c3)c2)nc1 ; c1cncc(-c2ccccc2-c2ccc3c(c2)c2cccnc2n3-c2cccc(-n3c4ccc(-c5ccccc5-c5cccnc5)cc4c4cccnc43)c2)c1 ; c1cncc(-c2cccc(-c3ccc4c(c3)c3cccnc3n4-c3cccc(-n4c5ccc(-c6cccc(-c7cccnc7)c6)cc5c5cccnc54)c3)c2)c1 ; c1ccc(-c2cccc(-c3ccc4c(c3)c3ccncc3n4-c3cccc(-n4c5ccc(-c6cccc(-c7ccccn7)c6)cc5c5ccncc54)c3)c2)nc1 ; c1cncc(-c2cccc(-c3ccc4c(c3)c3ccncc3n4-c3cccc(-n4c5ccc(-c6cccc(-c7cccnc7)c6)cc5c5ccncc54)c3)c2)c1]

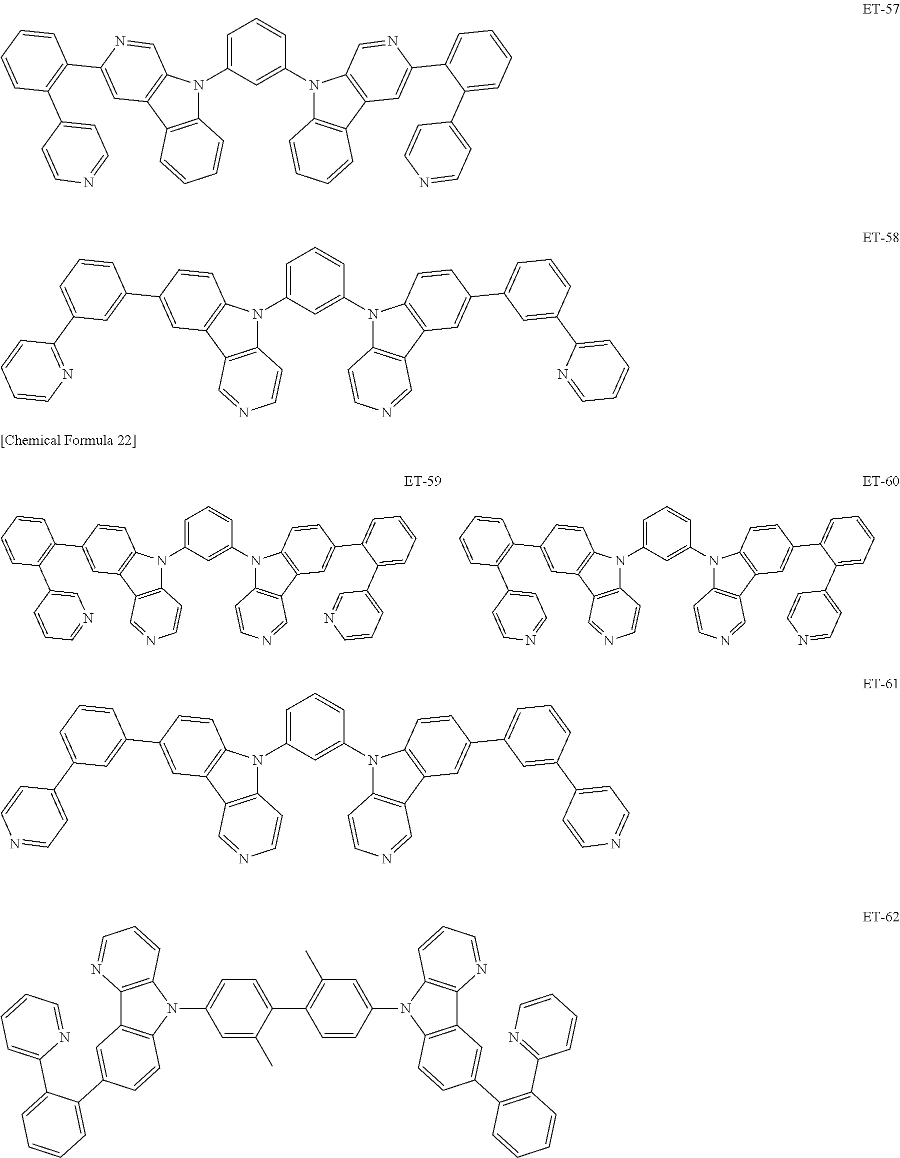

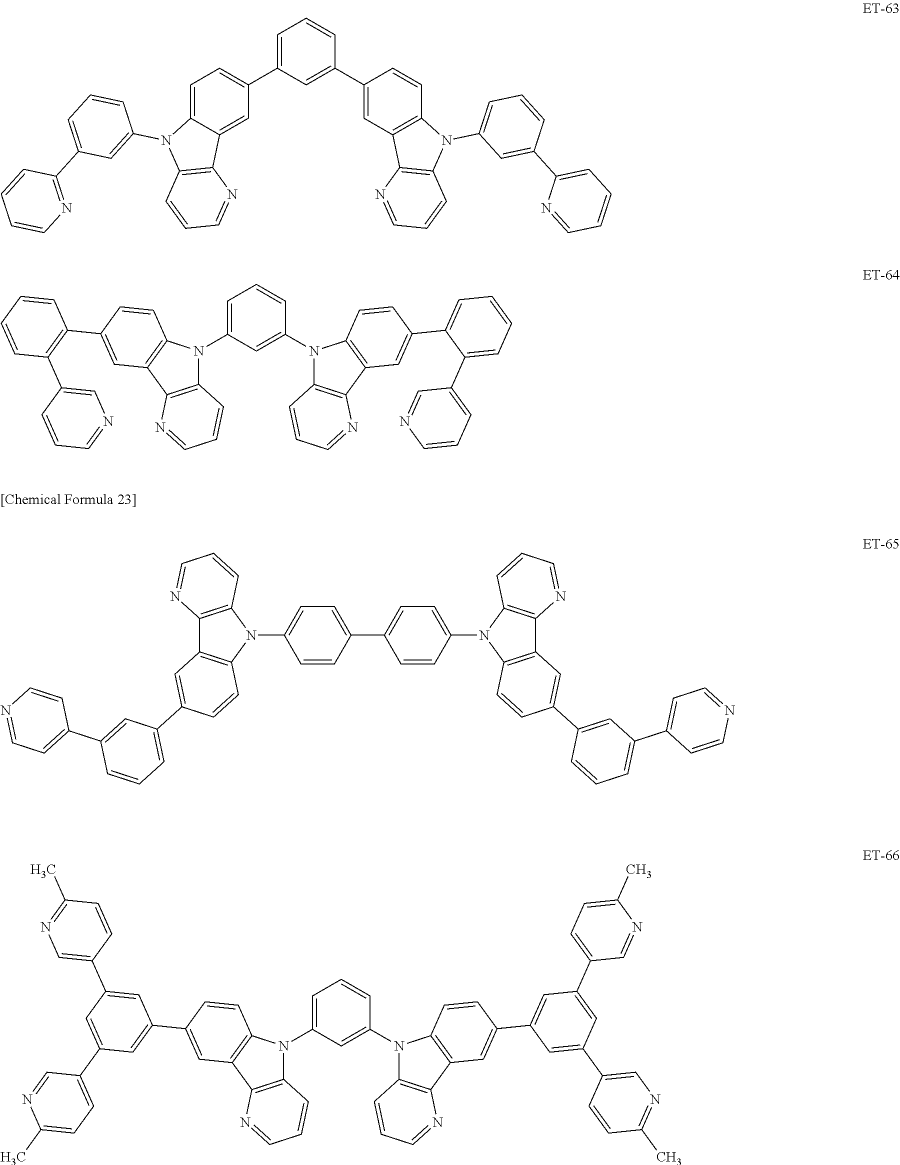

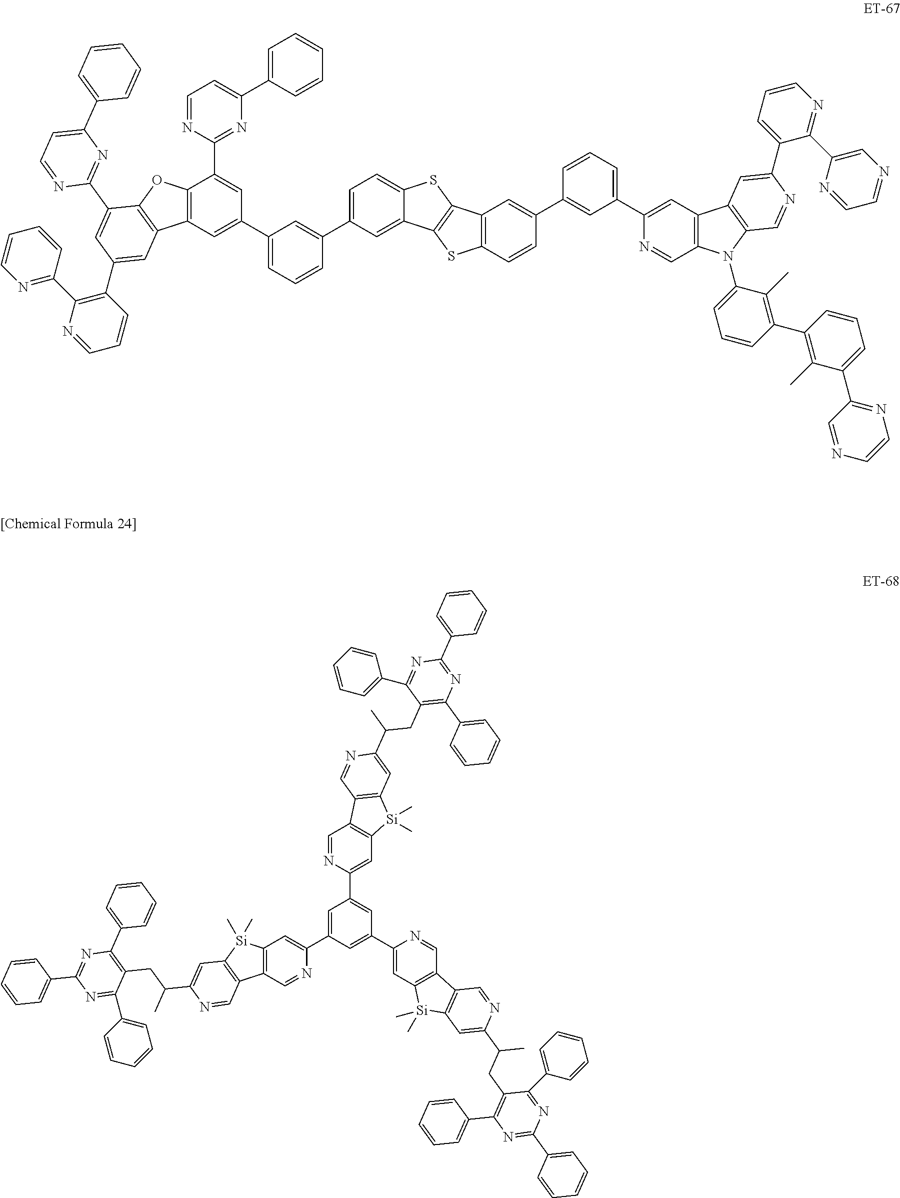 [SMILES: Cc1c(-c2cnccn2)cccc1-c1cccc(-n2c3cnc(-c4cccc(-c5ccc6sc7c8cc(-c9cccc(-c%10cc(-c%11nccc(-c%12ccccc%12)n%11)c%11oc%12c(-c%13nccc(-c%14ccccc%14)n%13)cc(-c%13cccnc%13-c%13ccccn%13)cc%12c%11c%10)c9)ccc8sc7c6c5)c4)cc3c3cc(-c4cccnc4-c4cnccn4)ncc32)c1C ; CC(Cc1c(-c2ccccc2)nc(-c2ccccc2)nc1-c1ccccc1)c1cc2c(cn1)-c1cnc(-c3cc(-c4cc5c(cn4)-c4cnc(C(C)Cc6c(-c7ccccc7)nc(-c7ccccc7)nc6-c6ccccc6)cc4[Si]5(C)C)cc(-c4cc5c(cn4)-c4cnc(C(C)Cc6c(-c7ccccc7)nc(-c7ccccc7)nc6-c6ccccc6)cc4[Si]5(C)C)c3)cc1[Si]2(C)C]

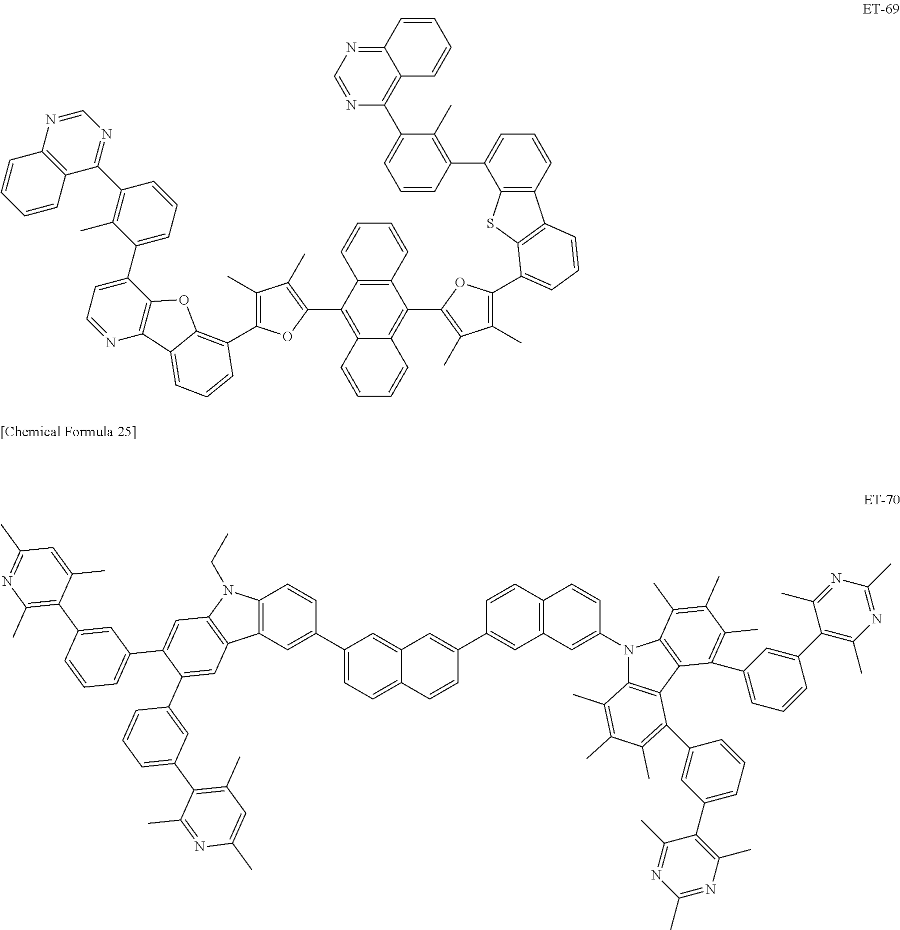

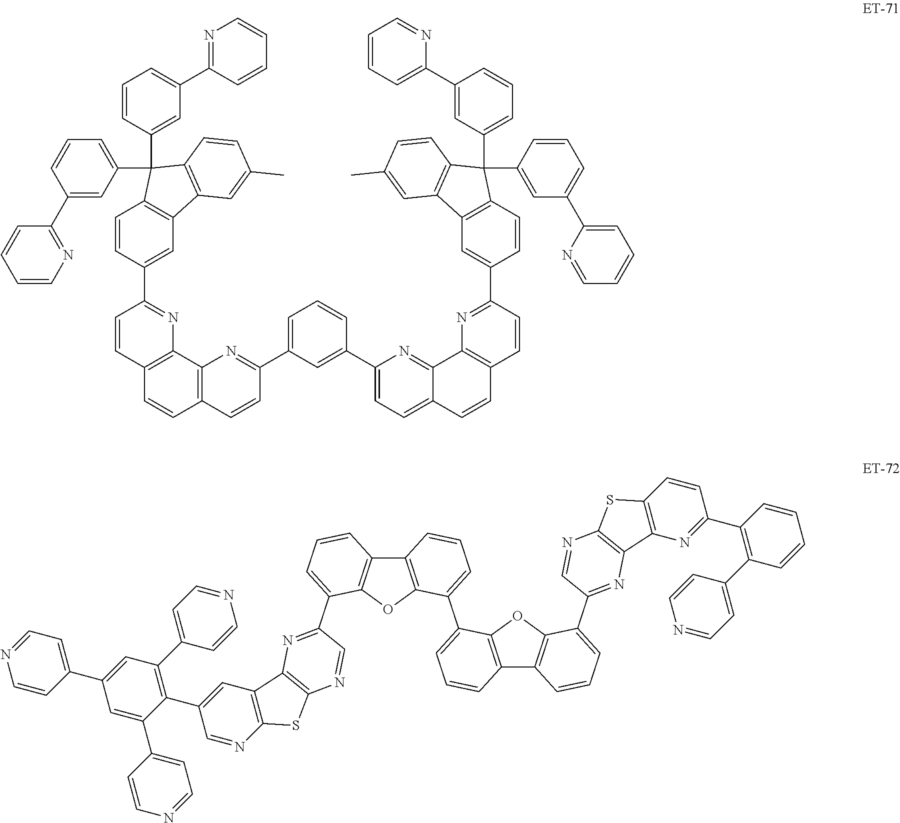 [SMILES: Cc1ccc2c(c1)-c1cc(-c3ccc4ccc5ccc(-c6cccc(-c7ccc8ccc9ccc(-c%10ccc%11c(c%10)-c%10cc(C)ccc%10C%11(c%10cccc(-c%11ccccn%11)c%10)c%10cccc(-c%11ccccn%11)c%10)nc9c8n7)c6)nc5c4n3)ccc1C2(c1cccc(-c2ccccn2)c1)c1cccc(-c2ccccn2)c1 ; c1ccc(-c2ccc3sc4ncc(-c5cccc6c5oc5c(-c7cccc8c7oc7c(-c9cnc%10sc%11ncc(-c%12c(-c%13ccncc%13)cc(-c%13ccncc%13)cc%12-c%12ccncc%12)cc%11c%10n9)cccc78)cccc56)nc4c3n2)c(-c2ccncc2)c1]

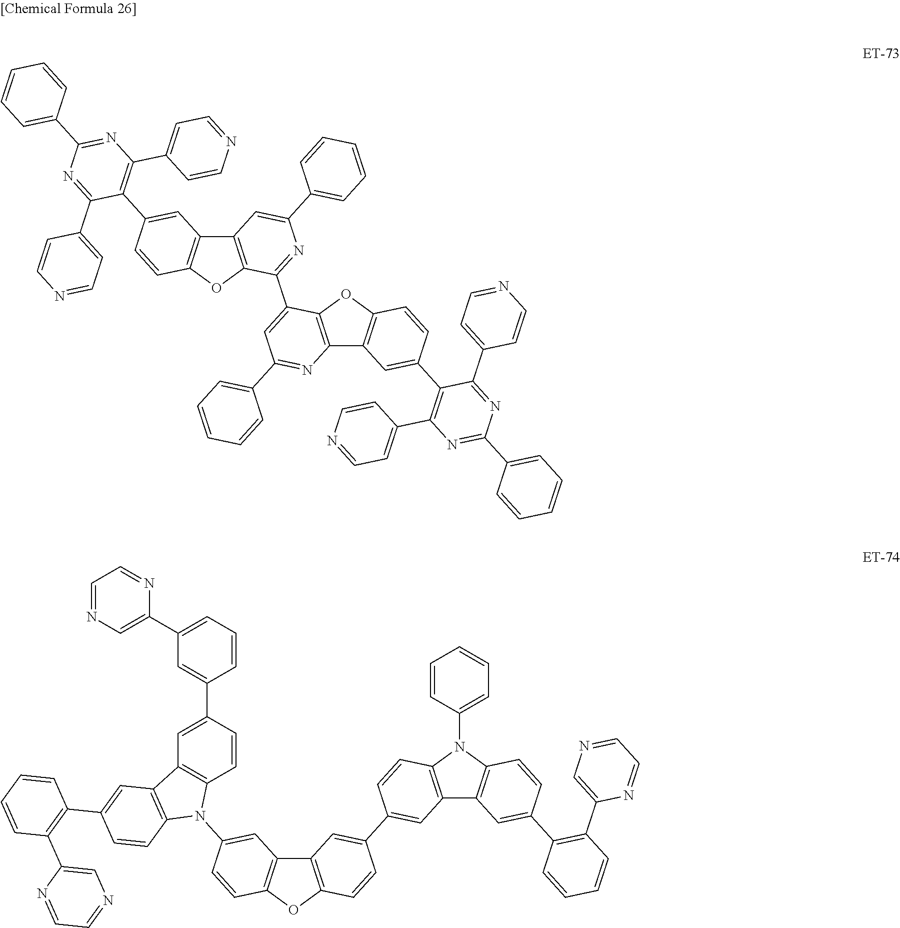 [SMILES: c1ccc(-c2cc3c(oc4ccc(-c5c(-c6ccncc6)nc(-c6ccccc6)nc5-c5ccncc5)cc43)c(-c3cc(-c4ccccc4)nc4c3oc3ccc(-c5c(-c6ccncc6)nc(-c6ccccc6)nc5-c5ccncc5)cc34)n2)cc1 ; c1ccc(-n2c3ccc(-c4ccc5oc6ccc(-n7c8ccc(-c9cccc(-c%10cnccn%10)c9)cc8c8cc(-c9ccccc9-c9cnccn9)ccc87)cc6c5c4)cc3c3cc(-c4ccccc4-c4cnccn4)ccc32)cc1]

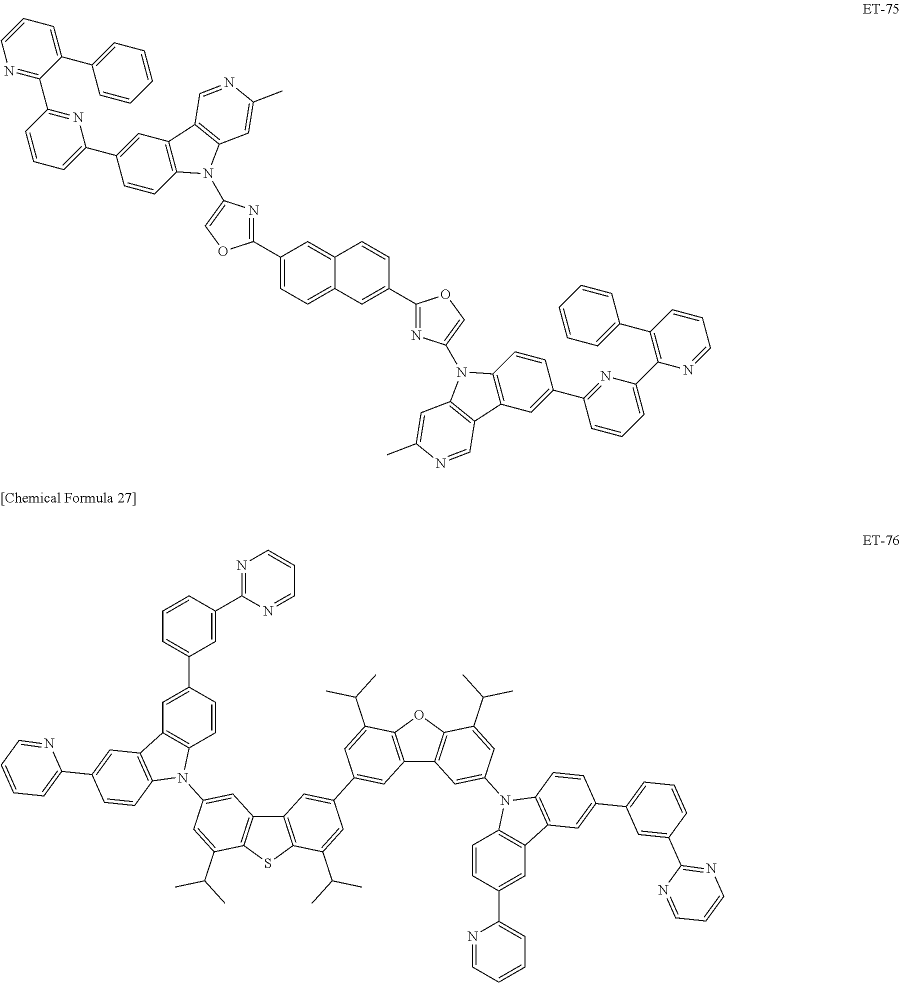 [SMILES: Cc1cc2c(cn1)c1cc(-c3cccc(-c4ncccc4-c4ccccc4)n3)ccc1n2-c1coc(-c2ccc3cc(-c4nc(-n5c6ccc(-c7cccc(-c8ncccc8-c8ccccc8)n7)cc6c6cnc(C)cc65)co4)ccc3c2)n1 ; CC(C)c1cc(-c2cc(C(C)C)c3sc4c(C(C)C)cc(-n5c6ccc(-c7cccc(-c8ncccn8)c7)cc6c6cc(-c7ccccn7)ccc65)cc4c3c2)cc2c1oc1c(C(C)C)cc(-n3c4ccc(-c5cccc(-c6ncccn6)c5)cc4c4cc(-c5ccccn5)ccc43)cc12]

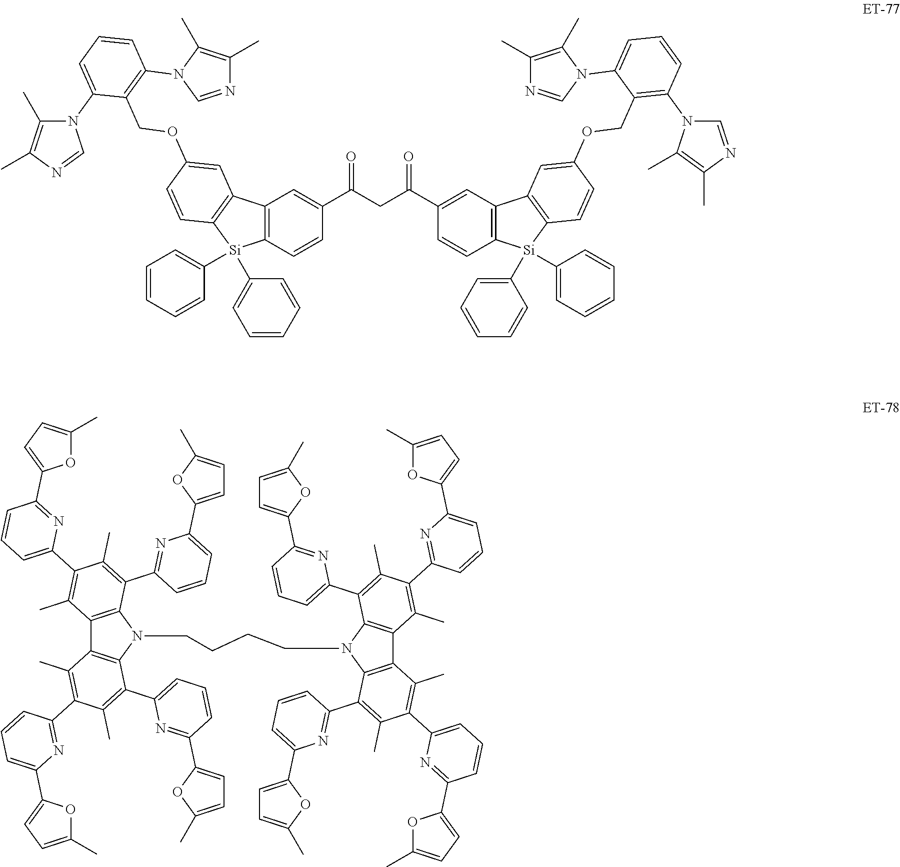 [SMILES: Cc1ncn(-c2cccc(-n3cnc(C)c3C)c2COc2ccc3c(c2)-c2cc(C(=O)CC(=O)c4ccc5c(c4)-c4cc(OCc6c(-n7cnc(C)c7C)cccc6-n6cnc(C)c6C)ccc4[Si]5(c4ccccc4)c4ccccc4)ccc2[Si]3(c2ccccc2)c2ccccc2)c1C ; Cc1ccc(-c2cccc(-c3c(C)c(-c4cccc(-c5ccc(C)o5)n4)c4c(c3C)c3c(C)c(-c5cccc(-c6ccc(C)o6)n5)c(C)c(-c5cccc(-c6ccc(C)o6)n5)c3n4CCCCn3c4c(-c5cccc(-c6ccc(C)o6)n5)c(C)c(-c5cccc(-c6ccc(C)o6)n5)c(C)c4c4c(C)c(-c5cccc(-c6ccc(C)o6)n5)c(C)c(-c5cccc(-c6ccc(C)o6)n5)c43)n2)o1]

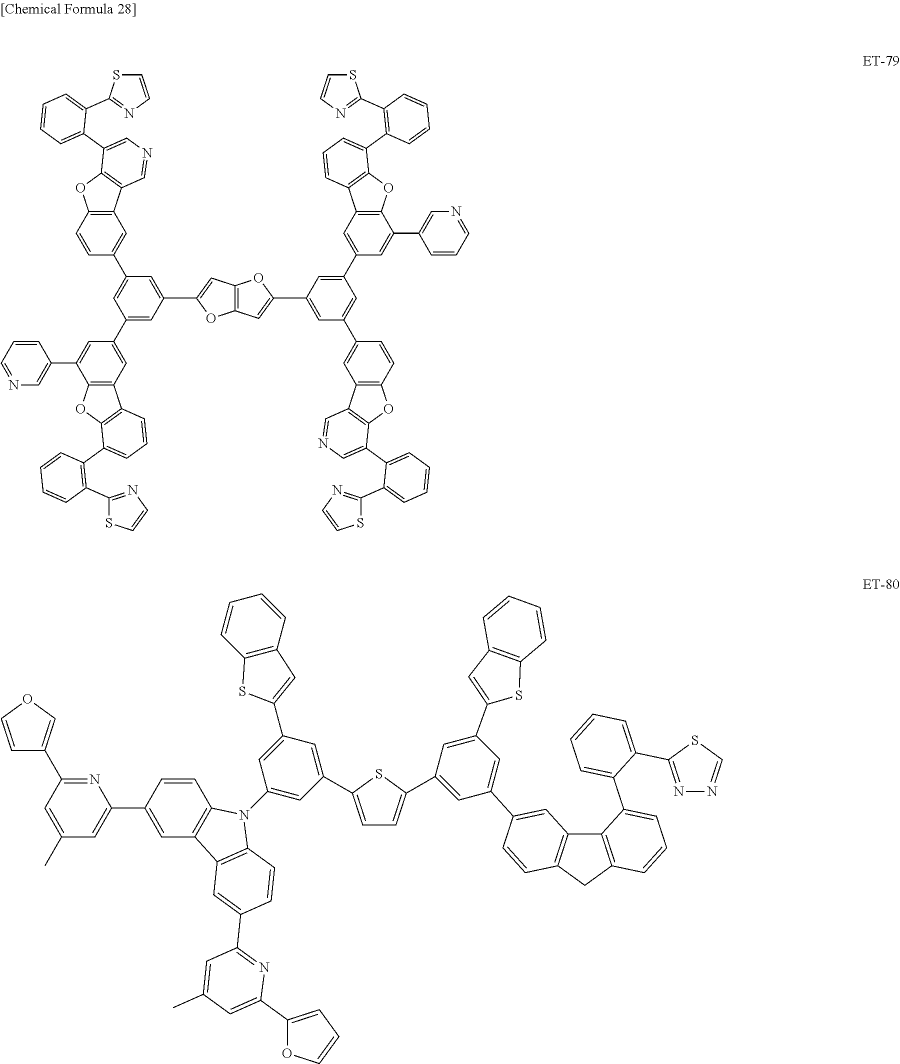 [SMILES: c1cncc(-c2cc(-c3cc(-c4ccc5oc6c(-c7ccccc7-c7nccs7)cncc6c5c4)cc(-c4cc5oc(-c6cc(-c7ccc8oc9c(-c%10ccccc%10-c%10nccs%10)cncc9c8c7)cc(-c7cc(-c8cccnc8)c8oc9c(-c%10ccccc%10-c%10nccs%10)cccc9c8c7)c6)cc5o4)c3)cc3c2oc2c(-c4ccccc4-c4nccs4)cccc23)c1 ; Cc1cc(-c2ccoc2)nc(-c2ccc3c(c2)c2cc(-c4cc(C)cc(-c5ccco5)n4)ccc2n3-c2cc(-c3ccc(-c4cc(-c5ccc6c(c5)-c5c(cccc5-c5ccccc5-c5nncs5)C6)cc(-c5cc6ccccc6s5)c4)s3)cc(-c3cc4ccccc4s3)c2)c1]

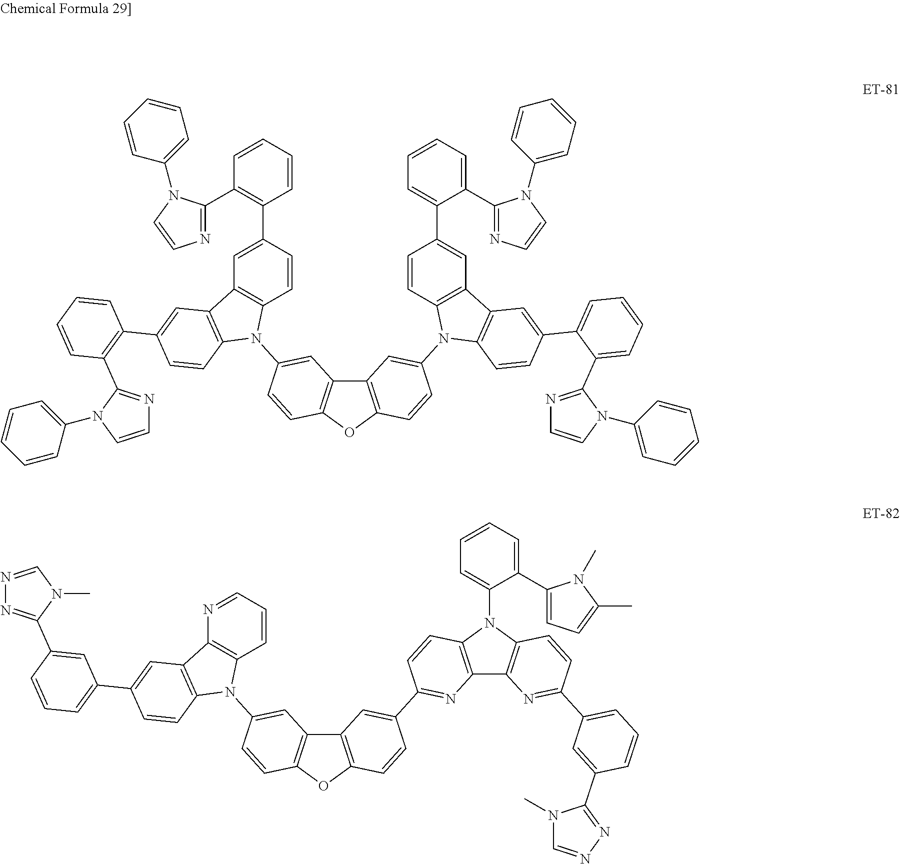 [SMILES: c1ccc(-n2ccnc2-c2ccccc2-c2ccc3c(c2)c2cc(-c4ccccc4-c4nccn4-c4ccccc4)ccc2n3-c2ccc3oc4ccc(-n5c6ccc(-c7ccccc7-c7nccn7-c7ccccc7)cc6c6cc(-c7ccccc7-c7nccn7-c7ccccc7)ccc65)cc4c3c2)cc1 ; Cc1ccc(-c2ccccc2-n2c3ccc(-c4cccc(-c5nncn5C)c4)nc3c3nc(-c4ccc5oc6ccc(-n7c8ccc(-c9cccc(-c%10nncn%10C)c9)cc8c8ncccc87)cc6c5c4)ccc32)n1C]

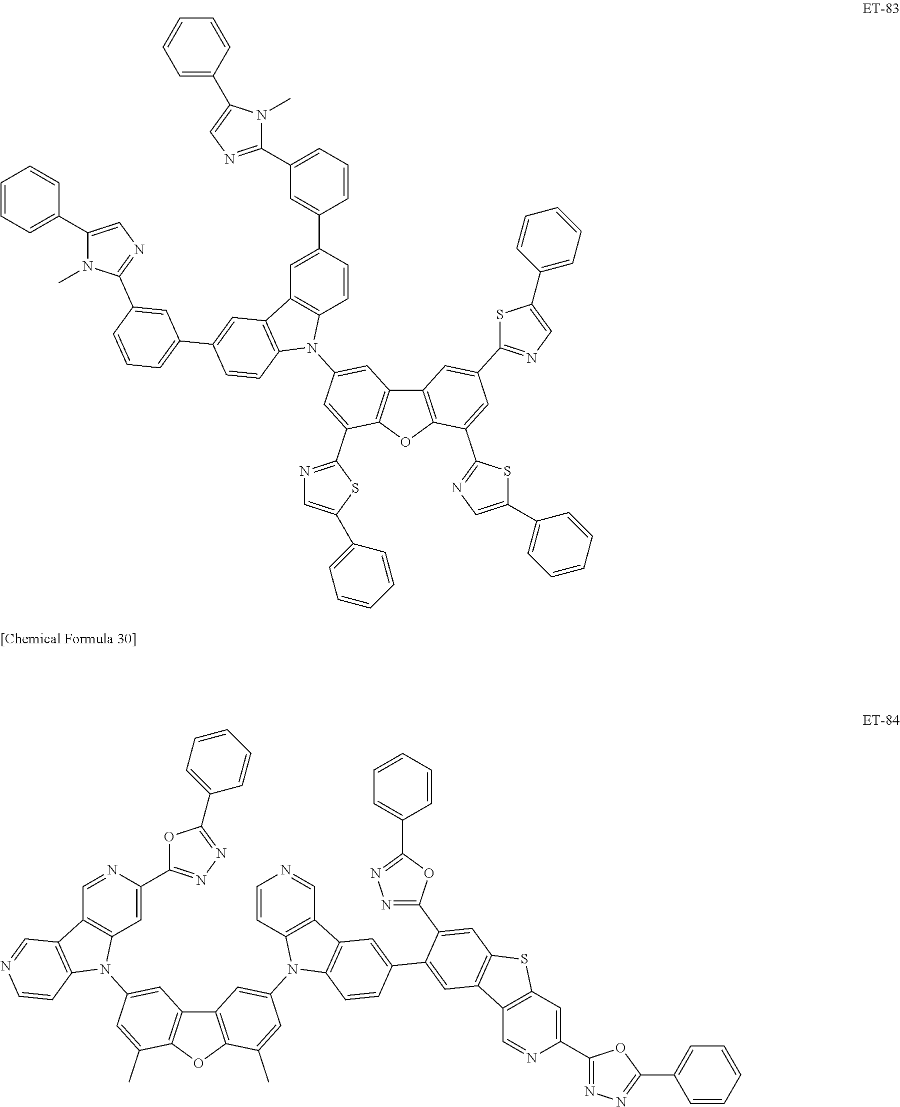 [SMILES: Cn1c(-c2ccccc2)cnc1-c1cccc(-c2ccc3c(c2)c2cc(-c4cccc(-c5ncc(-c6ccccc6)n5C)c4)ccc2n3-c2cc(-c3ncc(-c4ccccc4)s3)c3oc4c(-c5ncc(-c6ccccc6)s5)cc(-c5ncc(-c6ccccc6)s5)cc4c3c2)c1 ; Cc1cc(-n2c3ccncc3c3cc(-c4cc5c(cc4-c4nnc(-c6ccccc6)o4)sc4cc(-c6nnc(-c7ccccc7)o6)ncc45)ccc32)cc2c1oc1c(C)cc(-n3c4ccncc4c4cnc(-c5nnc(-c6ccccc6)o5)cc43)cc12]

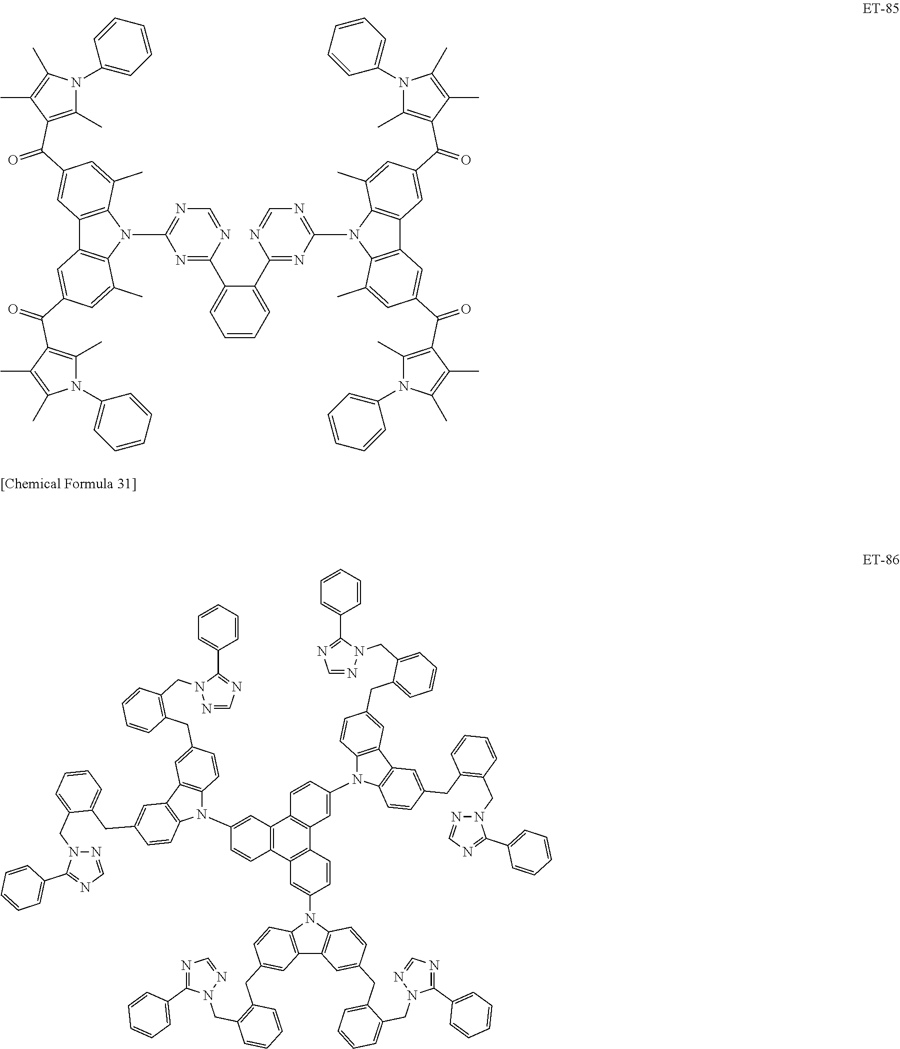

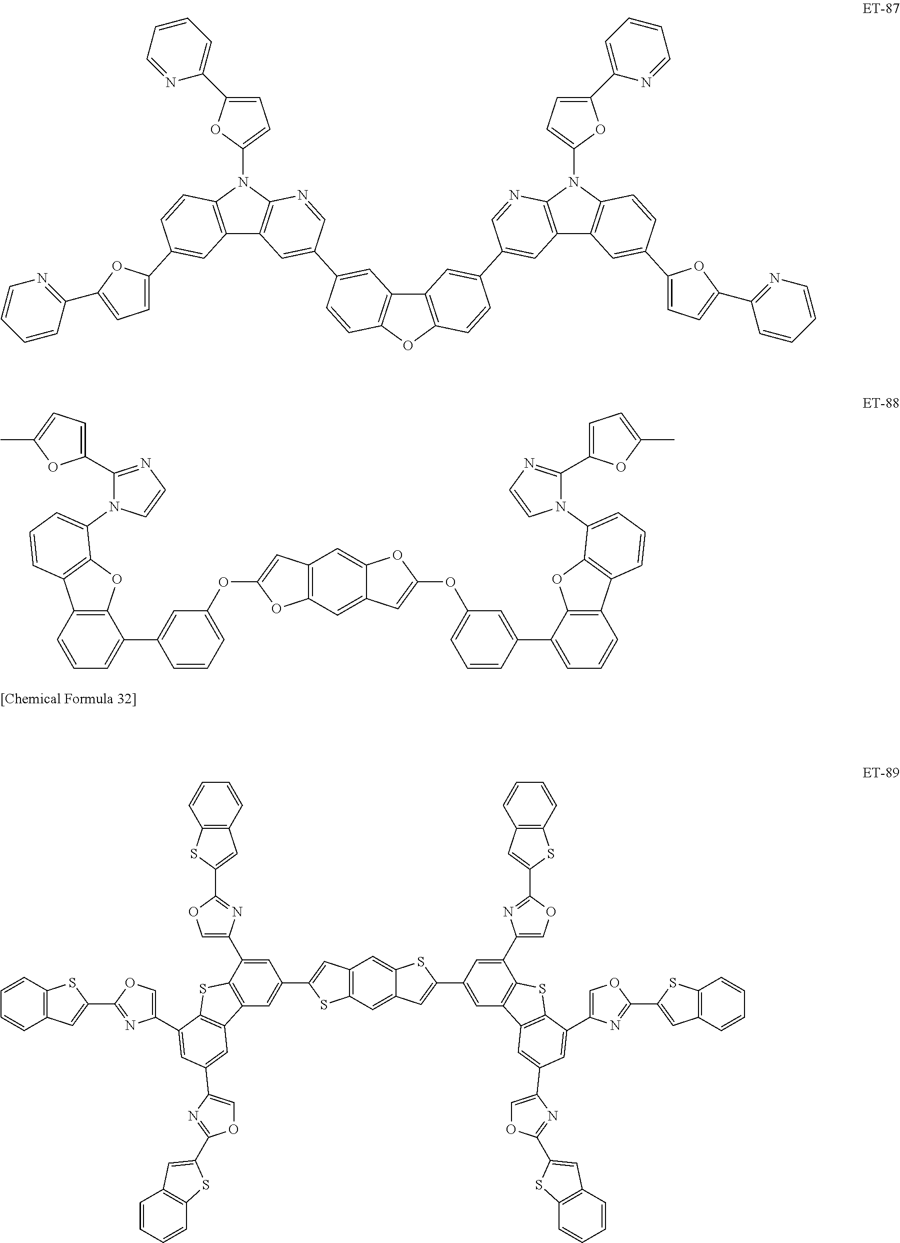

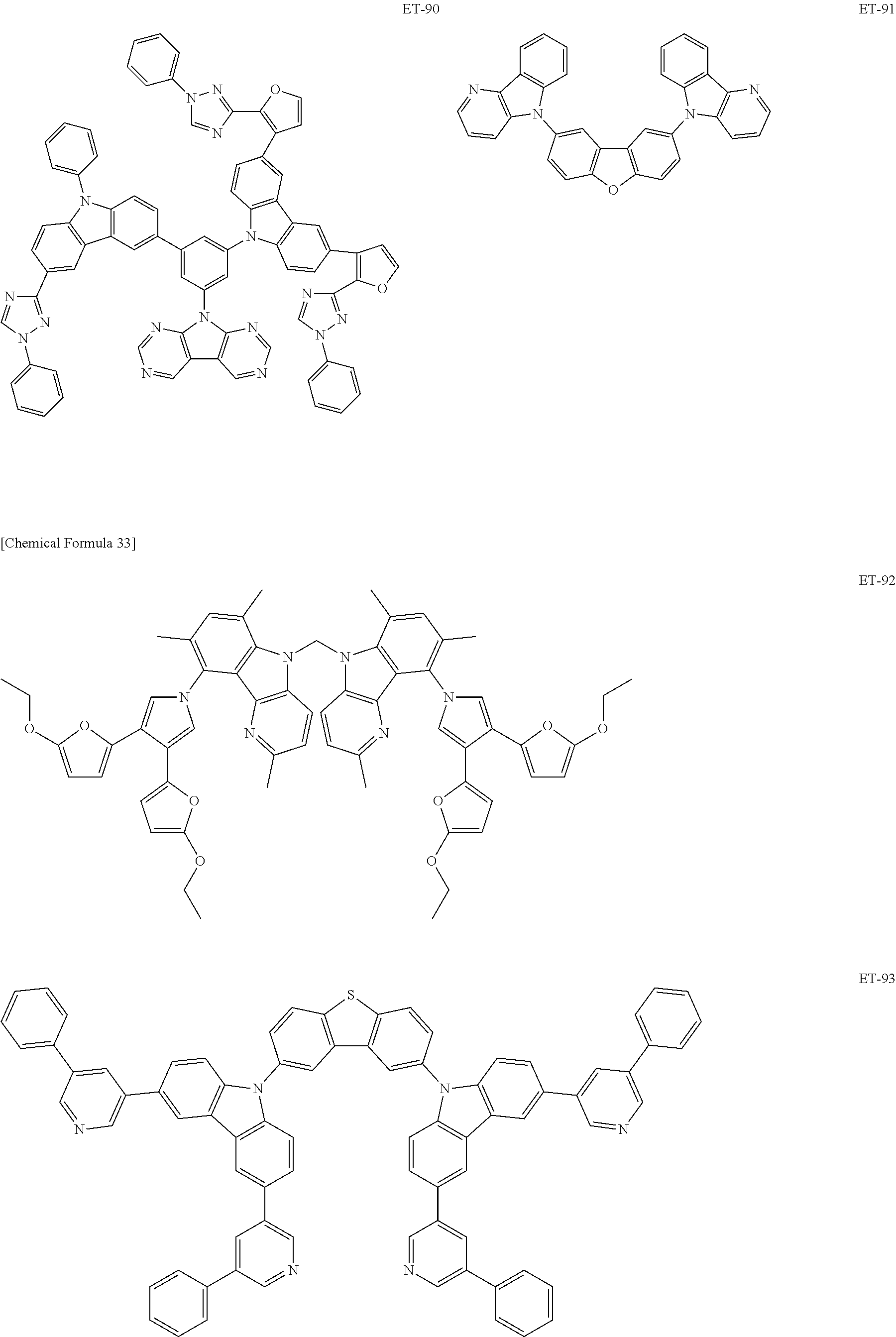

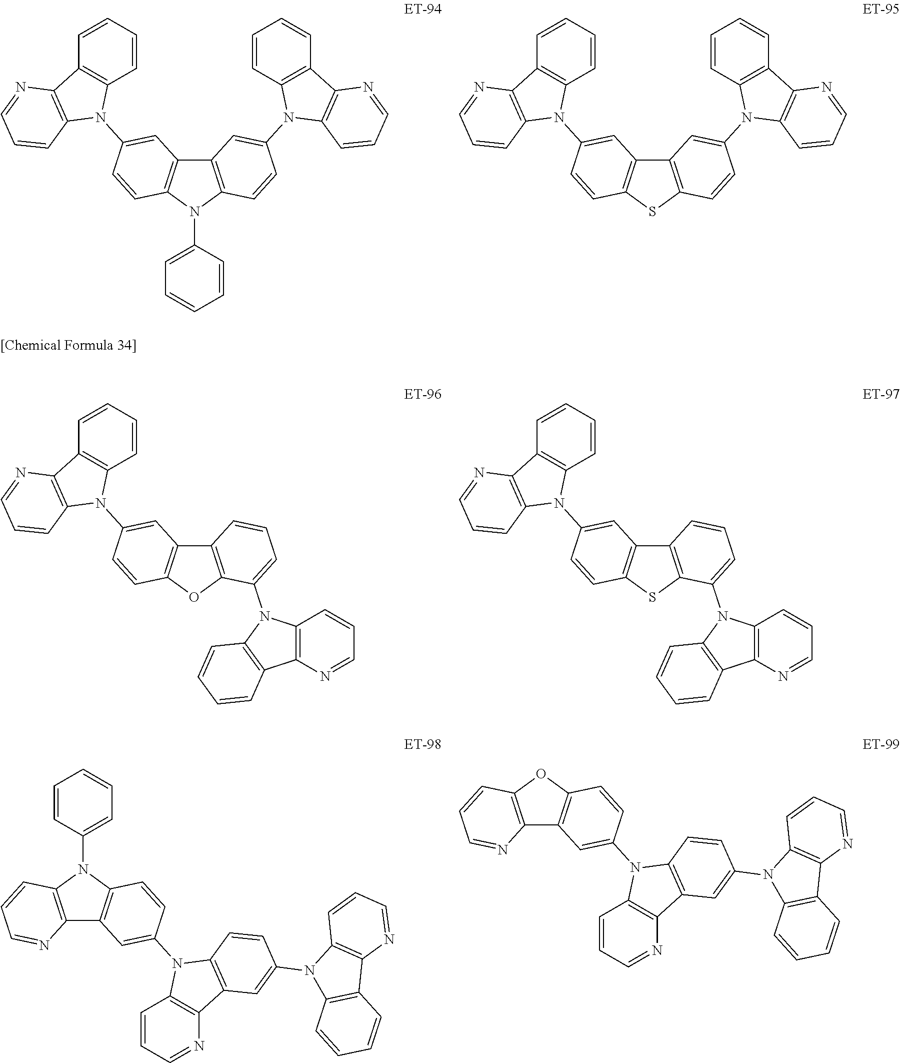 [SMILES: c1ccc(-n2c3ccc(-n4c5ccccc5c5ncccc54)cc3c3cc(-n4c5ccccc5c5ncccc54)ccc32)cc1 ; c1ccc2c(c1)c1ncccc1n2-c1ccc2sc3ccc(-n4c5ccccc5c5ncccc54)cc3c2c1 ; c1cc(-n2c3ccccc3c3ncccc32)c2oc3ccc(-n4c5ccccc5c5ncccc54)cc3c2c1 ; c1cc(-n2c3ccccc3c3ncccc32)c2sc3ccc(-n4c5ccccc5c5ncccc54)cc3c2c1 ; c1ccc(-n2c3ccc(-n4c5ccc(-n6c7ccccc7c7ncccc76)cc5c5ncccc54)cc3c3ncccc32)cc1 ; c1cnc2c(c1)oc1ccc(-n3c4ccc(-n5c6ccccc6c6ncccc65)cc4c4ncccc43)cc12]

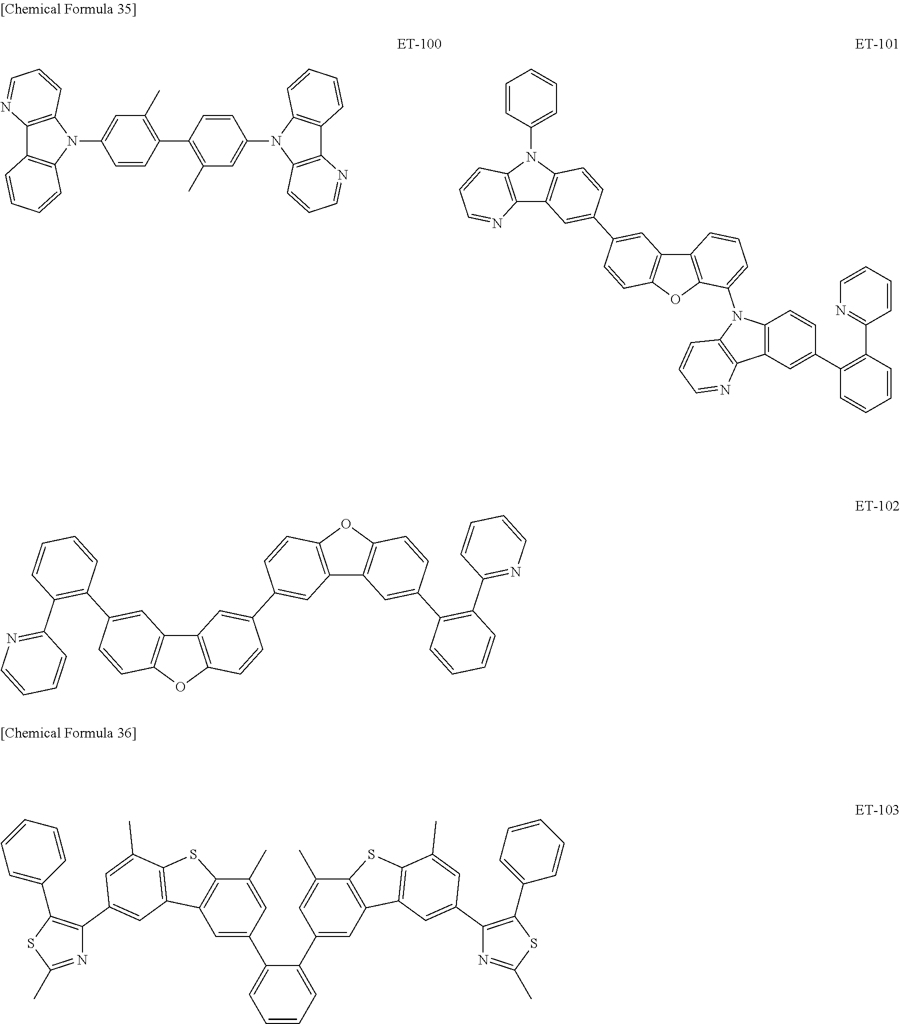 [SMILES: Cc1cc(-n2c3ccccc3c3ncccc32)ccc1-c1ccc(-n2c3ccccc3c3ncccc32)cc1C ; c1ccc(-n2c3ccc(-c4ccc5oc6c(-n7c8ccc(-c9ccccc9-c9ccccn9)cc8c8ncccc87)cccc6c5c4)cc3c3ncccc32)cc1 ; c1ccc(-c2ccccc2-c2ccc3oc4ccc(-c5ccc6oc7ccc(-c8ccccc8-c8ccccn8)cc7c6c5)cc4c3c2)nc1 ; Cc1nc(-c2cc(C)c3sc4c(C)cc(-c5ccccc5-c5cc(C)c6sc7c(C)cc(-c8nc(C)sc8-c8ccccc8)cc7c6c5)cc4c3c2)c(-c2ccccc2)s1]

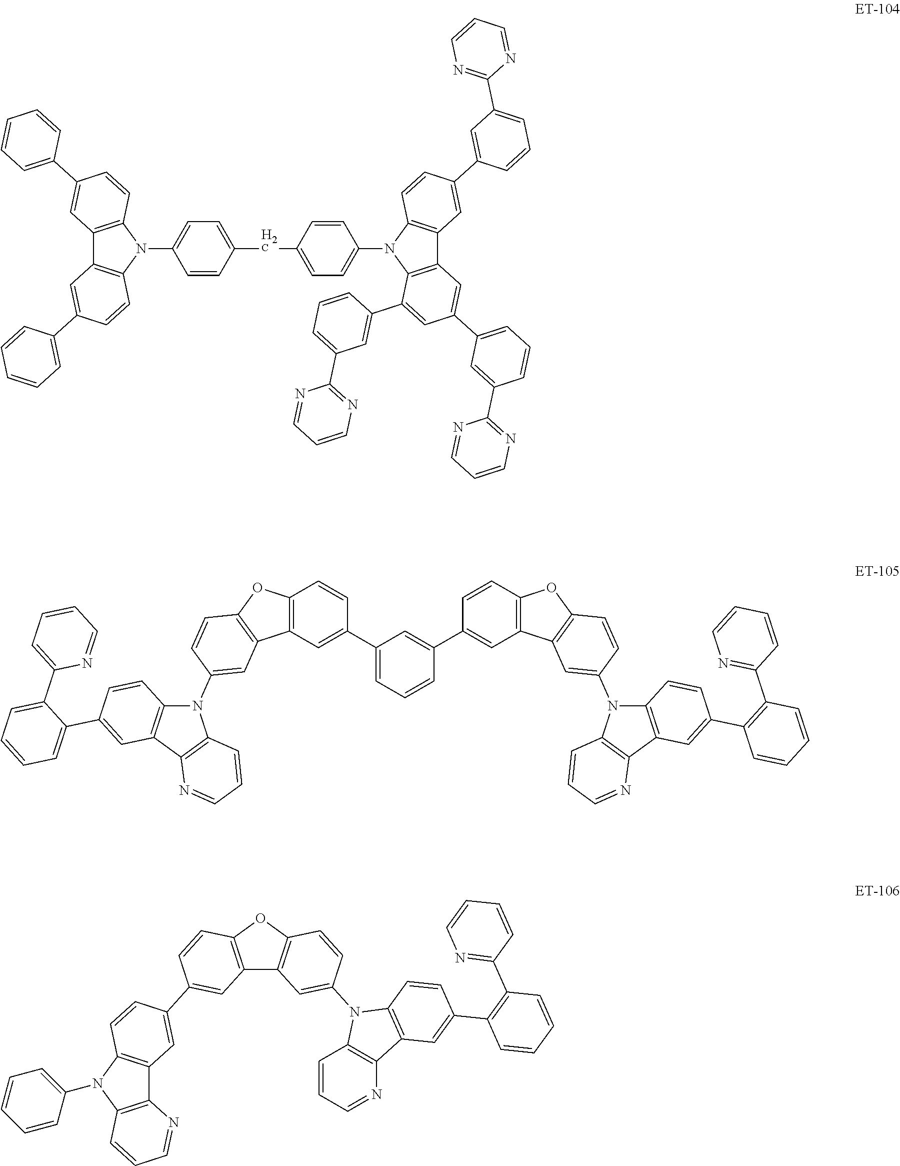 [SMILES: c1ccc(-c2ccc3c(c2)c2cc(-c4ccccc4)ccc2n3-c2ccc(Cc3ccc(-n4c5ccc(-c6cccc(-c7ncccn7)c6)cc5c5cc(-c6cccc(-c7ncccn7)c6)cc(-c6cccc(-c7ncccn7)c6)c54)cc3)cc2)cc1 ; c1ccc(-c2ccccc2-c2ccc3c(c2)c2ncccc2n3-c2ccc3oc4ccc(-c5cccc(-c6ccc7oc8ccc(-n9c%10ccc(-c%11ccccc%11-c%11ccccn%11)cc%10c%10ncccc%109)cc8c7c6)c5)cc4c3c2)nc1 ; c1ccc(-n2c3ccc(-c4ccc5oc6ccc(-n7c8ccc(-c9ccccc9-c9ccccn9)cc8c8ncccc87)cc6c5c4)cc3c3ncccc32)cc1]

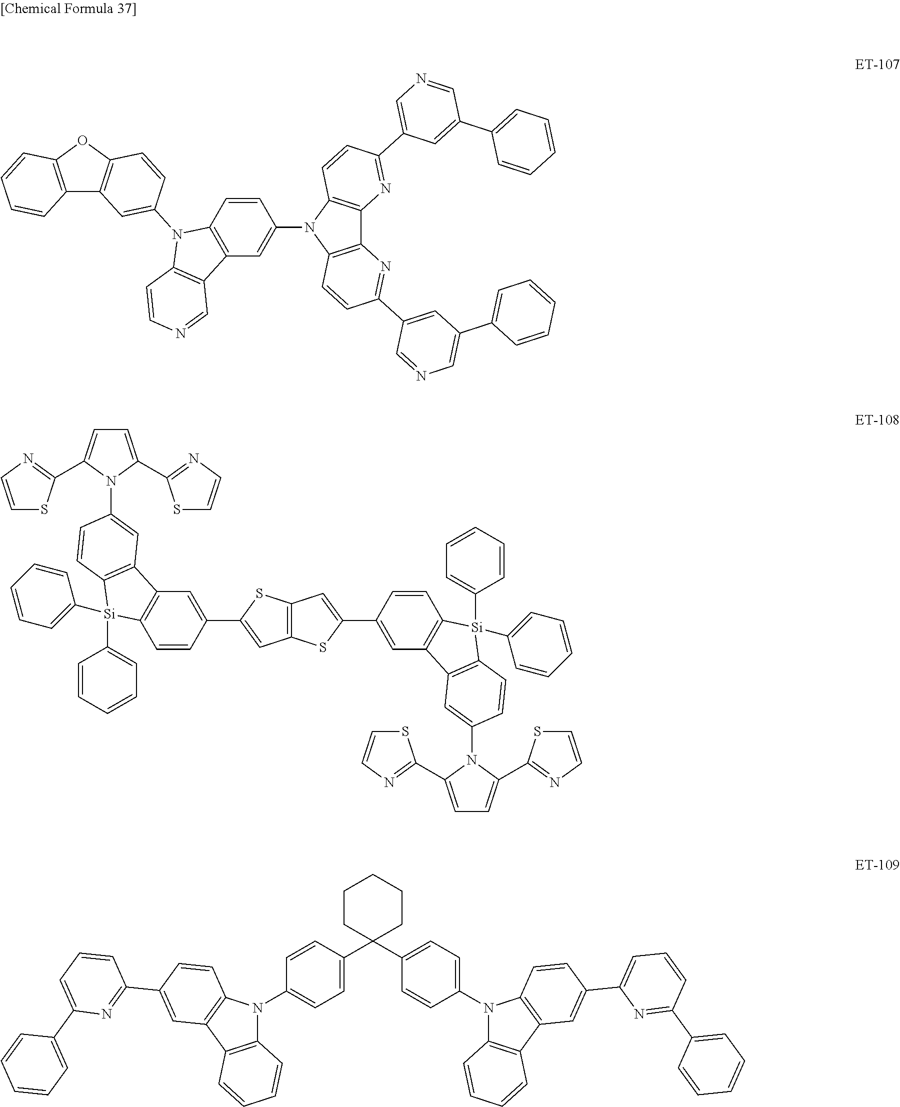 [SMILES: c1ccc(-c2cncc(-c3ccc4c(n3)c3nc(-c5cncc(-c6ccccc6)c5)ccc3n4-c3ccc4c(c3)c3cnccc3n4-c3ccc4oc5ccccc5c4c3)c2)cc1 ; c1ccc([Si]2(c3ccccc3)c3ccc(-c4cc5sc(-c6ccc7c(c6)-c6cc(-n8c(-c9nccs9)ccc8-c8nccs8)ccc6[Si]7(c6ccccc6)c6ccccc6)cc5s4)cc3-c3cc(-n4c(-c5nccs5)ccc4-c4nccs4)ccc32)cc1 ; c1ccc(-c2cccc(-c3ccc4c(c3)c3ccccc3n4-c3ccc(C4(c5ccc(-n6c7ccccc7c7cc(-c8cccc(-c9ccccc9)n8)ccc76)cc5)CCCCC4)cc3)n2)cc1]

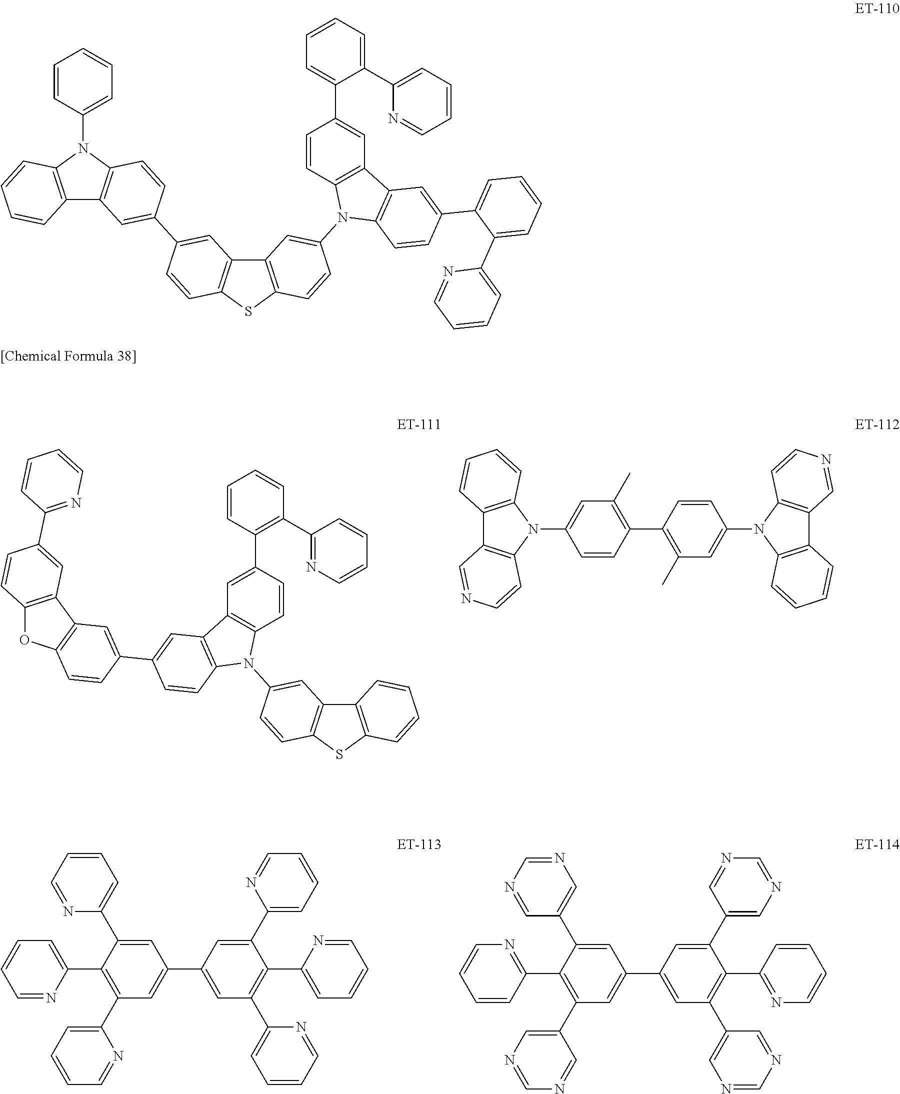 [SMILES: c1ccc(-n2c3ccccc3c3cc(-c4ccc5sc6ccc(-n7c8ccc(-c9ccccc9-c9ccccn9)cc8c8cc(-c9ccccc9-c9ccccn9)ccc87)cc6c5c4)ccc32)cc1 ; c1ccc(-c2ccc3oc4ccc(-c5ccc6c(c5)c5cc(-c7ccccc7-c7ccccn7)ccc5n6-c5ccc6sc7ccccc7c6c5)cc4c3c2)nc1 ; Cc1cc(-n2c3ccccc3c3cnccc32)ccc1-c1ccc(-n2c3ccccc3c3cnccc32)cc1C ; c1ccc(-c2cc(-c3cc(-c4ccccn4)c(-c4ccccn4)c(-c4ccccn4)c3)cc(-c3ccccn3)c2-c2ccccn2)nc1 ; c1ccc(-c2c(-c3cncnc3)cc(-c3cc(-c4cncnc4)c(-c4ccccn4)c(-c4cncnc4)c3)cc2-c2cncnc2)nc1]

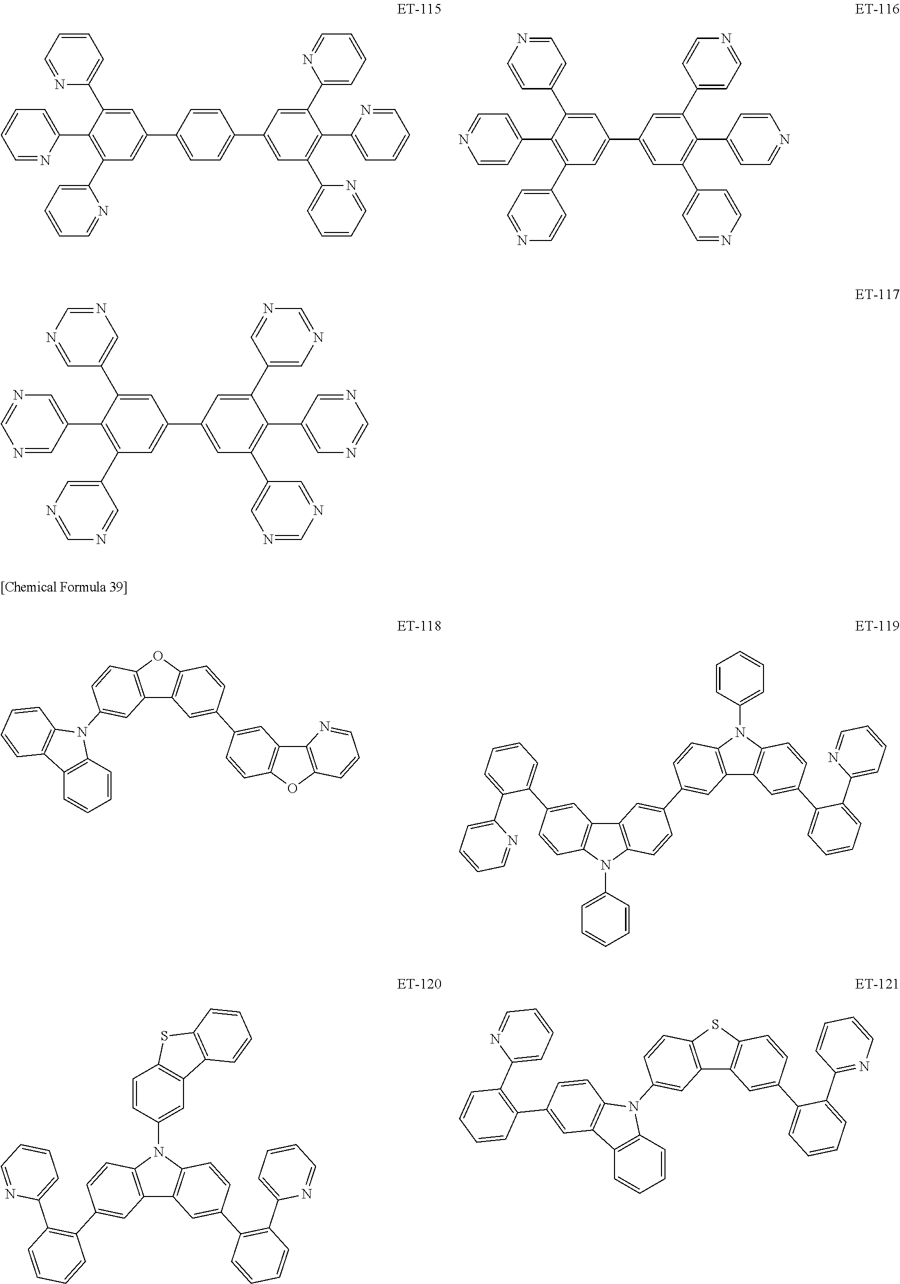

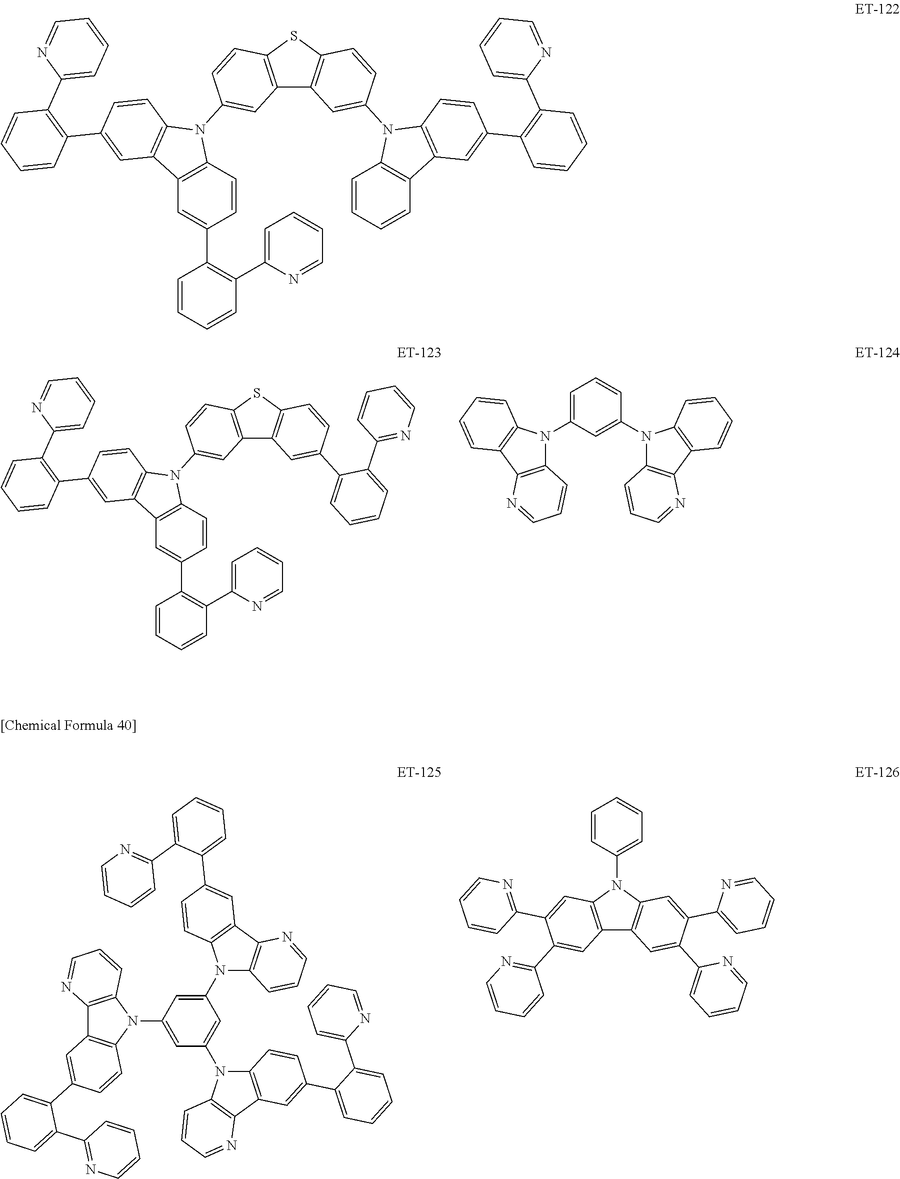 [SMILES: c1ccc(-c2ccccc2-c2ccc3c(c2)c2ccccc2n3-c2ccc3sc4ccc(-n5c6ccc(-c7ccccc7-c7ccccn7)cc6c6cc(-c7ccccc7-c7ccccn7)ccc65)cc4c3c2)nc1 ; c1ccc(-c2ccccc2-c2ccc3sc4ccc(-n5c6ccc(-c7ccccc7-c7ccccn7)cc6c6cc(-c7ccccc7-c7ccccn7)ccc65)cc4c3c2)nc1 ; c1cc(-n2c3ccccc3c3ncccc32)cc(-n2c3ccccc3c3ncccc32)c1 ; c1ccc(-c2ccccc2-c2ccc3c(c2)c2ncccc2n3-c2cc(-n3c4ccc(-c5ccccc5-c5ccccn5)cc4c4ncccc43)cc(-n3c4ccc(-c5ccccc5-c5ccccn5)cc4c4ncccc43)c2)nc1 ; c1ccc(-n2c3cc(-c4ccccn4)c(-c4ccccn4)cc3c3cc(-c4ccccn4)c(-c4ccccn4)cc32)cc1]

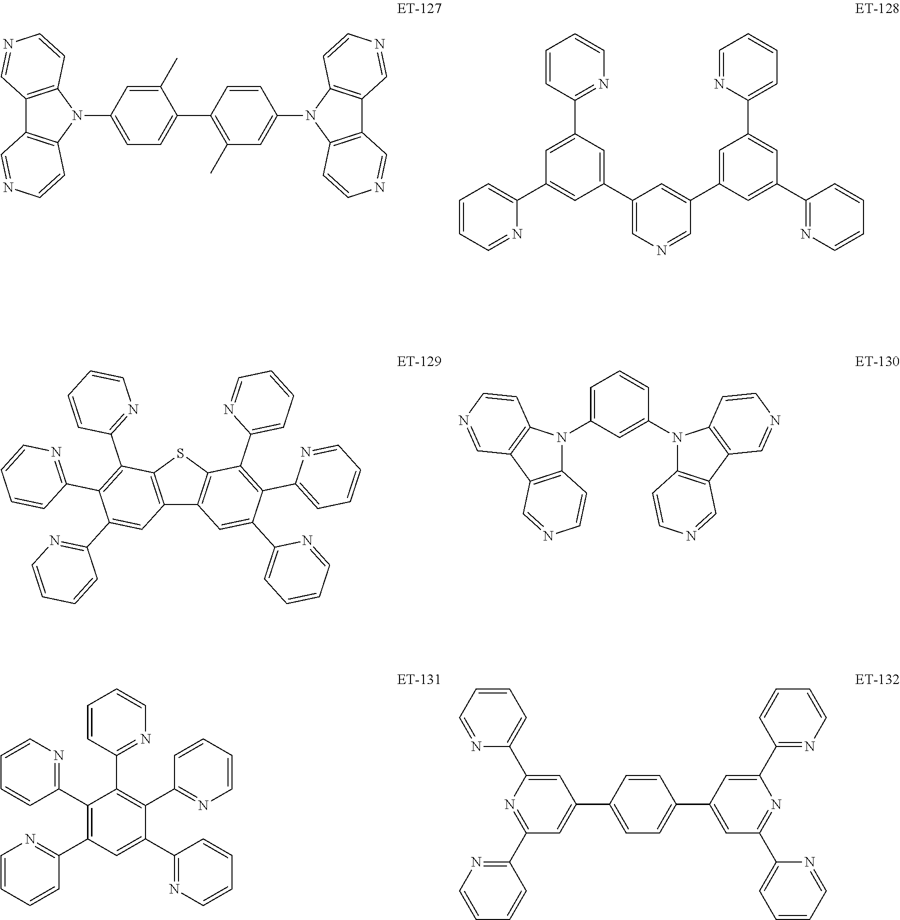 [SMILES: Cc1cc(-n2c3ccncc3c3cnccc32)ccc1-c1ccc(-n2c3ccncc3c3cnccc32)cc1C ; c1ccc(-c2cc(-c3cncc(-c4cc(-c5ccccn5)cc(-c5ccccn5)c4)c3)cc(-c3ccccn3)c2)nc1 ; c1ccc(-c2cc3c(sc4c(-c5ccccn5)c(-c5ccccn5)c(-c5ccccn5)cc43)c(-c3ccccn3)c2-c2ccccn2)nc1 ; c1cc(-n2c3ccncc3c3cnccc32)cc(-n2c3ccncc3c3cnccc32)c1 ; c1ccc(-c2cc(-c3ccccn3)c(-c3ccccn3)c(-c3ccccn3)c2-c2ccccn2)nc1 ; c1ccc(-c2cc(-c3ccc(-c4cc(-c5ccccn5)nc(-c5ccccn5)c4)cc3)cc(-c3ccccn3)n2)nc1]

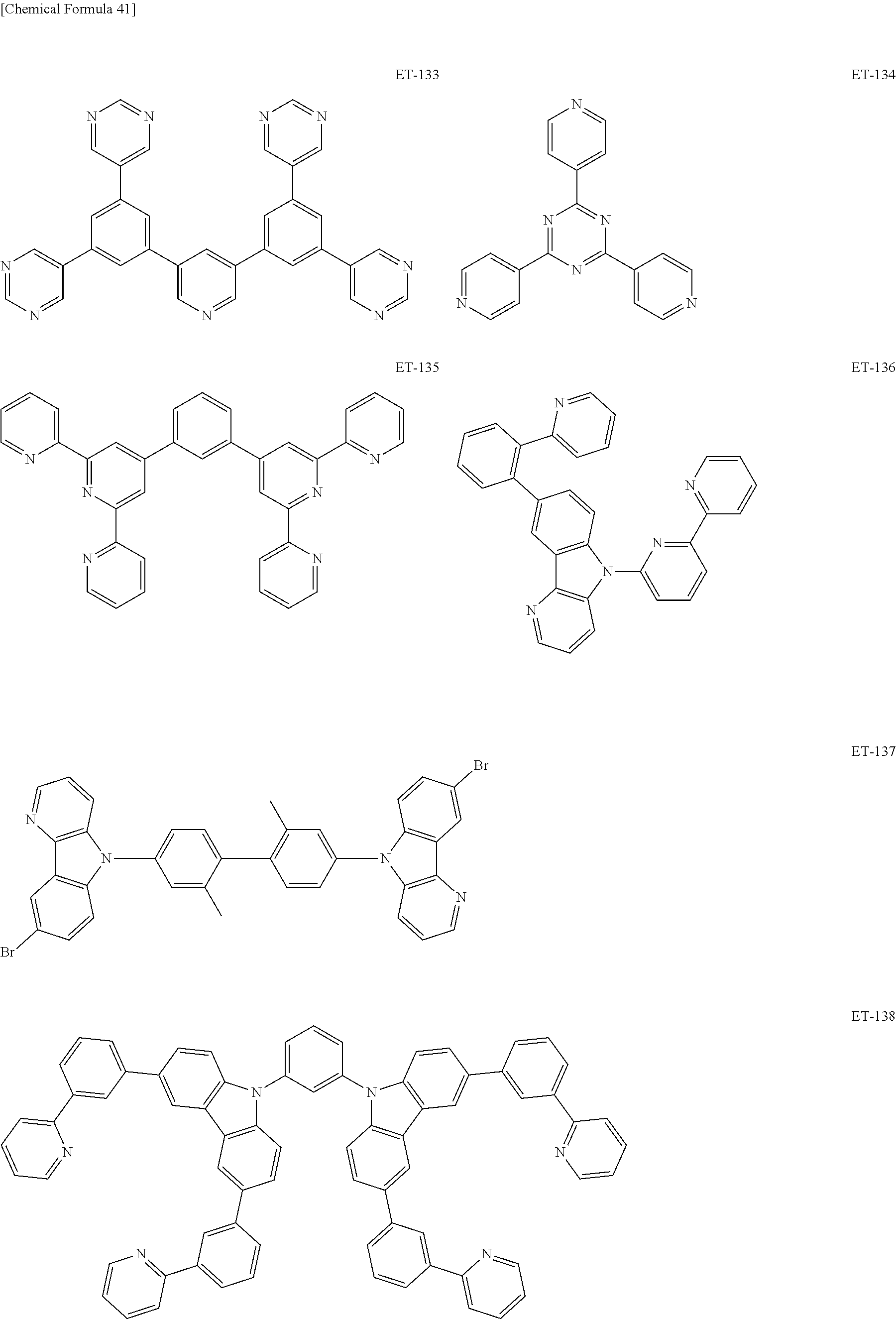 [SMILES: c1ncc(-c2cc(-c3cncnc3)cc(-c3cncc(-c4cc(-c5cncnc5)cc(-c5cncnc5)c4)c3)c2)cn1 ; c1cc(-c2nc(-c3ccncc3)nc(-c3ccncc3)n2)ccn1 ; c1ccc(-c2cc(-c3cccc(-c4cc(-c5ccccn5)nc(-c5ccccn5)c4)c3)cc(-c3ccccn3)n2)nc1 ; c1ccc(-c2cccc(-n3c4ccc(-c5ccccc5-c5ccccn5)cc4c4ncccc43)n2)nc1 ; Cc1cc(-n2c3ccc(Br)cc3c3ncccc32)ccc1-c1ccc(-n2c3ccc(Br)cc3c3ncccc32)cc1C ; c1ccc(-c2cccc(-c3ccc4c(c3)c3cc(-c5cccc(-c6ccccn6)c5)ccc3n4-c3cccc(-n4c5ccc(-c6cccc(-c7ccccn7)c6)cc5c5cc(-c6cccc(-c7ccccn7)c6)ccc54)c3)c2)nc1]

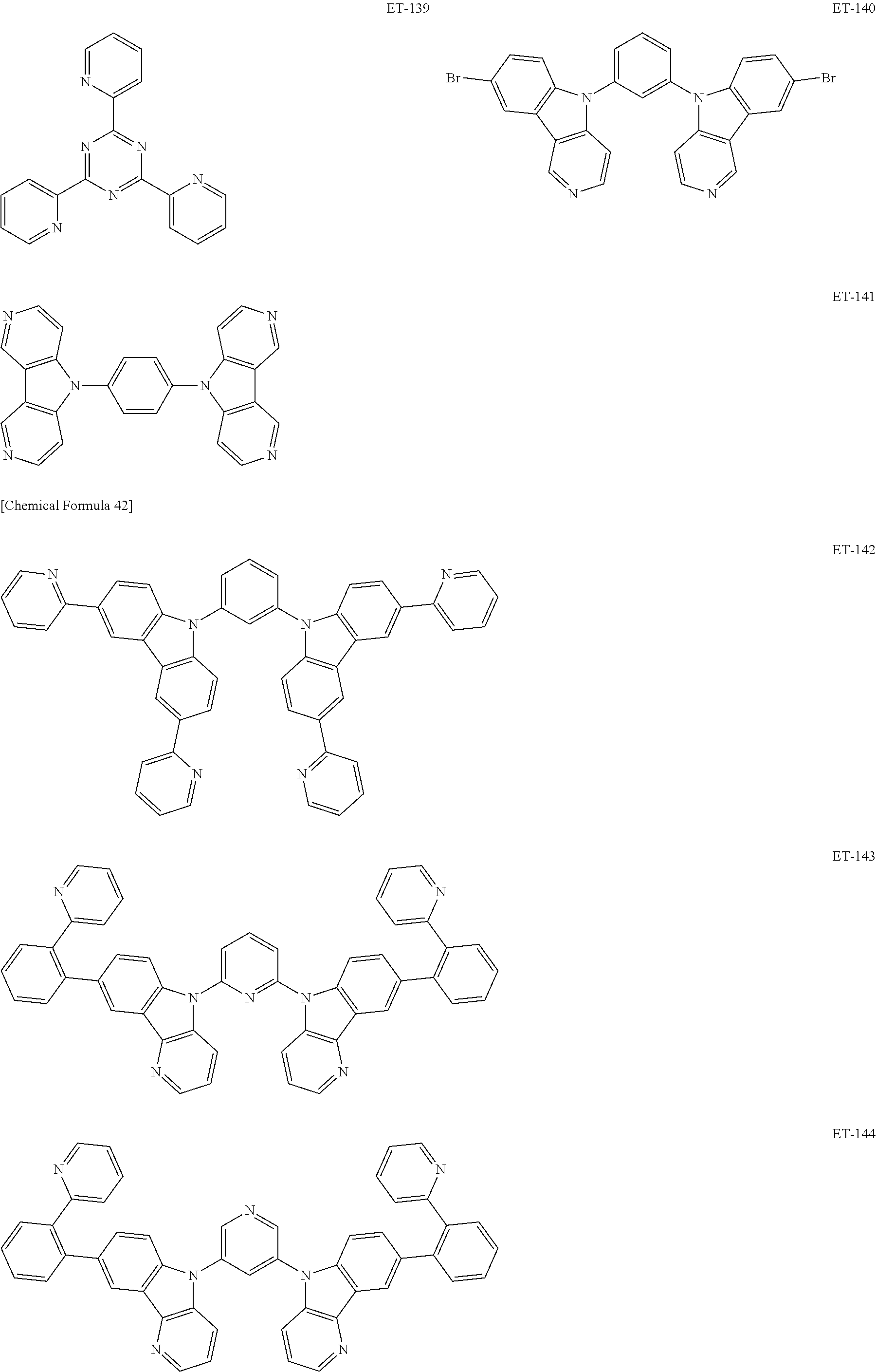 [SMILES: c1ccc(-c2nc(-c3ccccn3)nc(-c3ccccn3)n2)nc1 ; Brc1ccc2c(c1)c1cnccc1n2-c1cccc(-n2c3ccncc3c3cc(Br)ccc32)c1 ; c1cc2c(cn1)c1cnccc1n2-c1ccc(-n2c3ccncc3c3cnccc32)cc1 ; c1ccc(-c2ccc3c(c2)c2cc(-c4ccccn4)ccc2n3-c2cccc(-n3c4ccc(-c5ccccn5)cc4c4cc(-c5ccccn5)ccc43)c2)nc1 ; c1ccc(-c2ccccc2-c2ccc3c(c2)c2ncccc2n3-c2cccc(-n3c4ccc(-c5ccccc5-c5ccccn5)cc4c4ncccc43)n2)nc1 ; c1ccc(-c2ccccc2-c2ccc3c(c2)c2ncccc2n3-c2cncc(-n3c4ccc(-c5ccccc5-c5ccccn5)cc4c4ncccc43)c2)nc1]

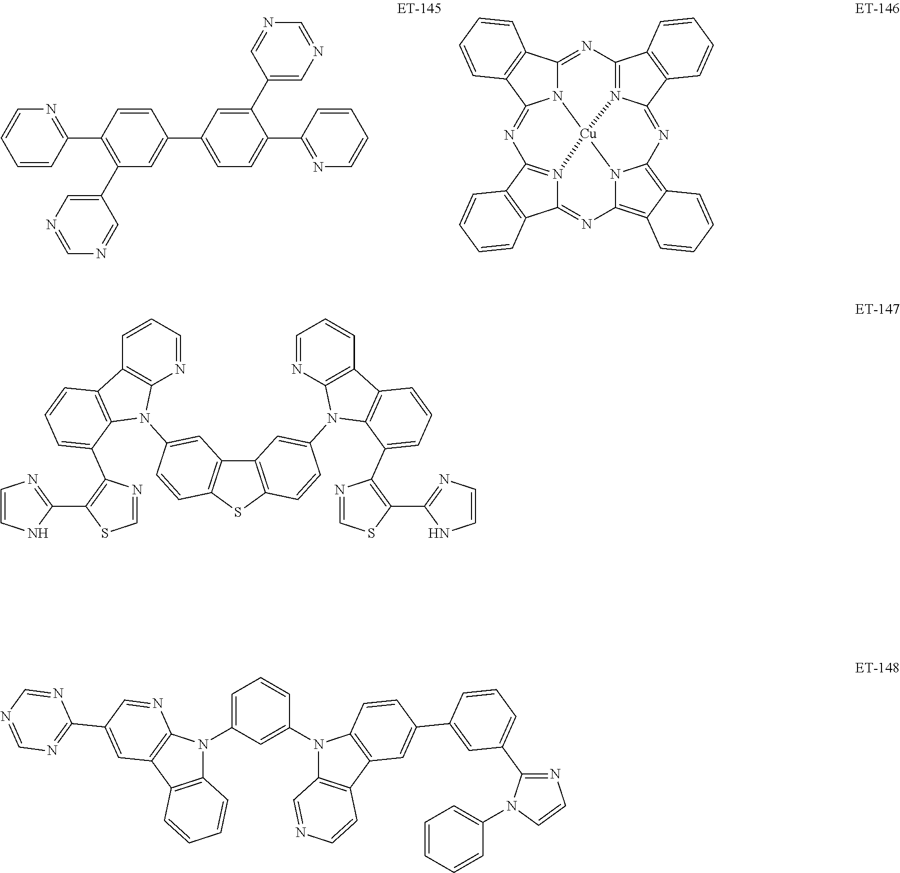

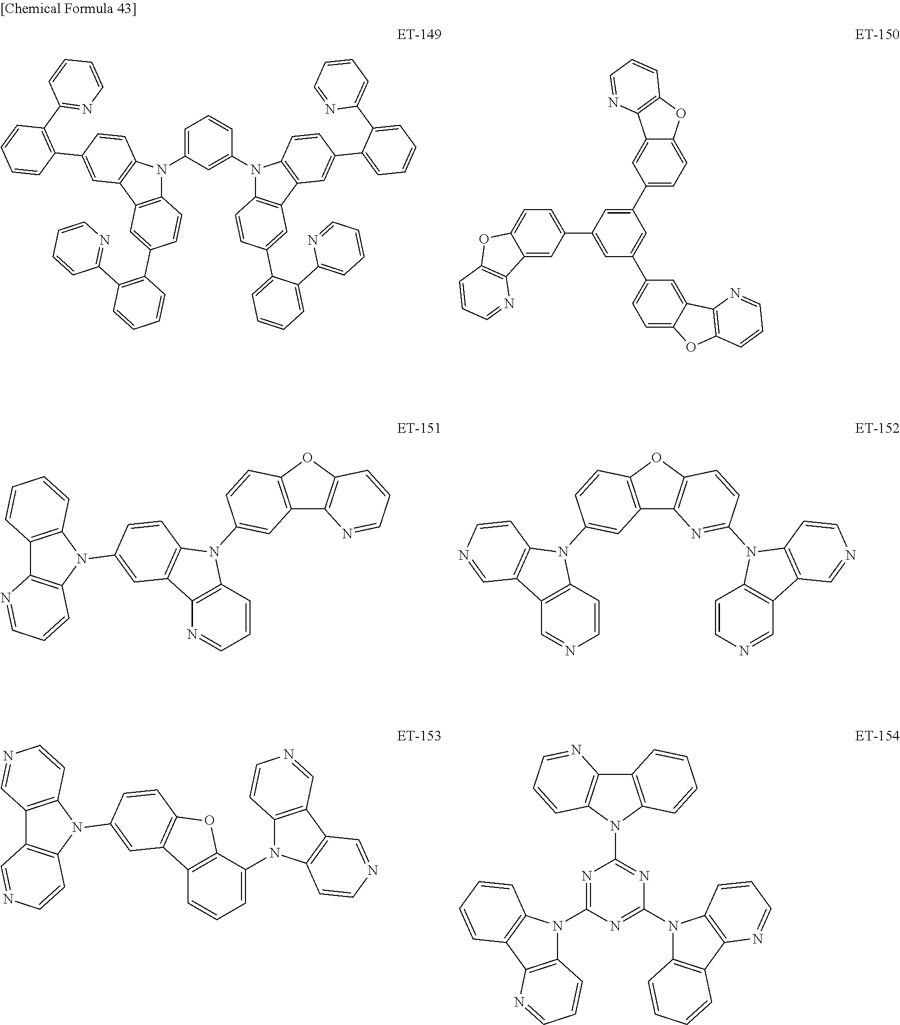 [SMILES: c1ccc(-c2ccccc2-c2ccc3c(c2)c2cc(-c4ccccc4-c4ccccn4)ccc2n3-c2cccc(-n3c4ccc(-c5ccccc5-c5ccccn5)cc4c4cc(-c5ccccc5-c5ccccn5)ccc43)c2)nc1 ; c1cnc2c(c1)oc1ccc(-c3cc(-c4ccc5oc6cccnc6c5c4)cc(-c4ccc5oc6cccnc6c5c4)c3)cc12 ; c1cnc2c(c1)oc1ccc(-n3c4ccc(-n5c6ccccc6c6ncccc65)cc4c4ncccc43)cc12 ; c1cc2c(cn1)c1cnccc1n2-c1ccc2oc3ccc(-n4c5ccncc5c5cnccc54)nc3c2c1 ; c1cc(-n2c3ccncc3c3cnccc32)c2oc3ccc(-n4c5ccncc5c5cnccc54)cc3c2c1 ; c1ccc2c(c1)c1ncccc1n2-c1nc(-n2c3ccccc3c3ncccc32)nc(-n2c3ccccc3c3ncccc32)n1]

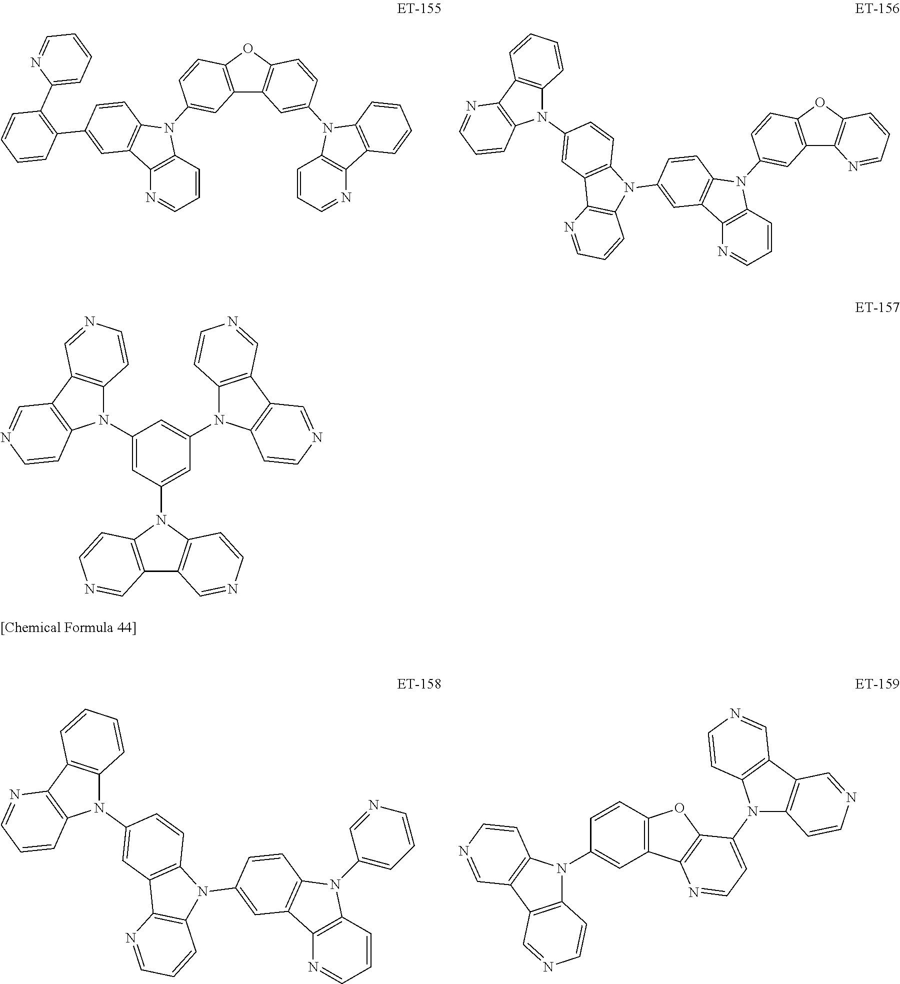 [SMILES: c1ccc(-c2ccccc2-c2ccc3c(c2)c2ncccc2n3-c2ccc3oc4ccc(-n5c6ccccc6c6ncccc65)cc4c3c2)nc1 ; c1cnc2c(c1)oc1ccc(-n3c4ccc(-n5c6ccc(-n7c8ccccc8c8ncccc87)cc6c6ncccc65)cc4c4ncccc43)cc12 ; c1cc2c(cn1)c1cnccc1n2-c1cc(-n2c3ccncc3c3cnccc32)cc(-n2c3ccncc3c3cnccc32)c1 ; c1cncc(-n2c3ccc(-n4c5ccc(-n6c7ccccc7c7ncccc76)cc5c5ncccc54)cc3c3ncccc32)c1 ; c1cc2c(cn1)c1cnccc1n2-c1ccc2oc3c(-n4c5ccncc5c5cnccc54)ccnc3c2c1]

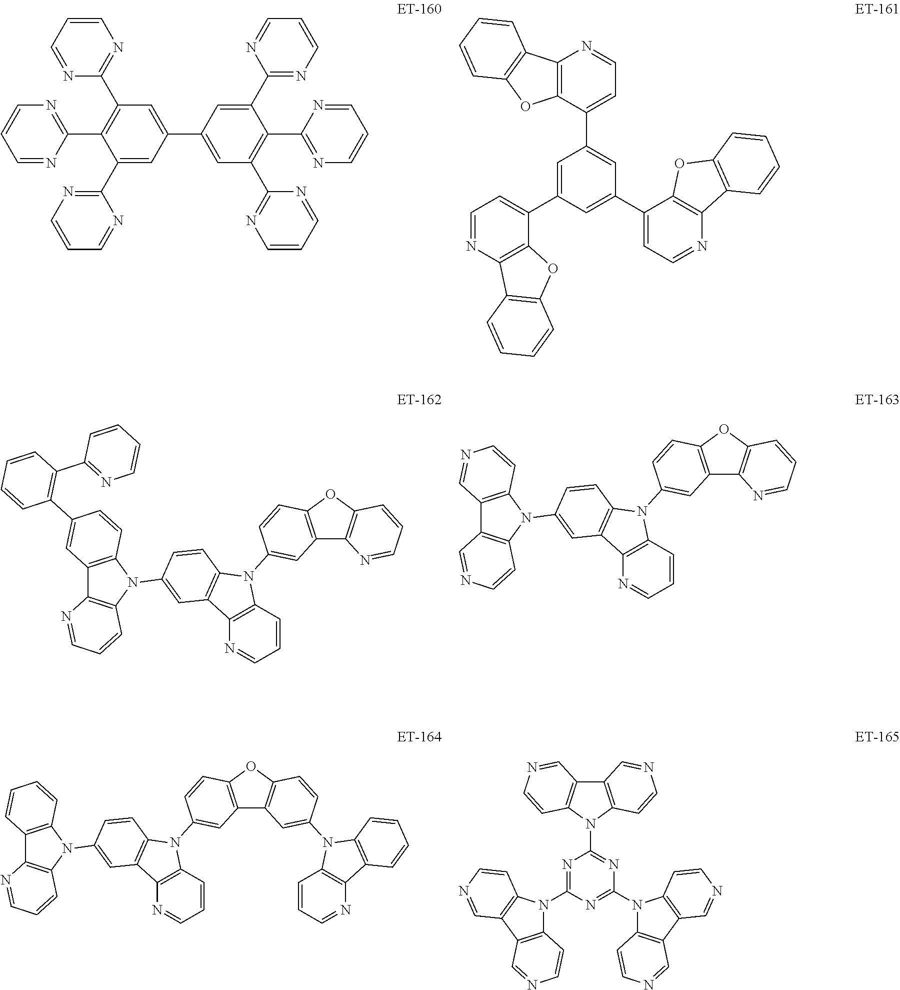 [SMILES: c1cnc(-c2cc(-c3cc(-c4ncccn4)c(-c4ncccn4)c(-c4ncccn4)c3)cc(-c3ncccn3)c2-c2ncccn2)nc1 ; c1ccc2c(c1)oc1c(-c3cc(-c4ccnc5c4oc4ccccc45)cc(-c4ccnc5c4oc4ccccc45)c3)ccnc12 ; c1ccc(-c2ccccc2-c2ccc3c(c2)c2ncccc2n3-c2ccc3c(c2)c2ncccc2n3-c2ccc3oc4cccnc4c3c2)nc1 ; c1cnc2c(c1)oc1ccc(-n3c4ccc(-n5c6ccncc6c6cnccc65)cc4c4ncccc43)cc12 ; c1ccc2c(c1)c1ncccc1n2-c1ccc2oc3ccc(-n4c5ccc(-n6c7ccccc7c7ncccc76)cc5c5ncccc54)cc3c2c1 ; c1cc2c(cn1)c1cnccc1n2-c1nc(-n2c3ccncc3c3cnccc32)nc(-n2c3ccncc3c3cnccc32)n1]

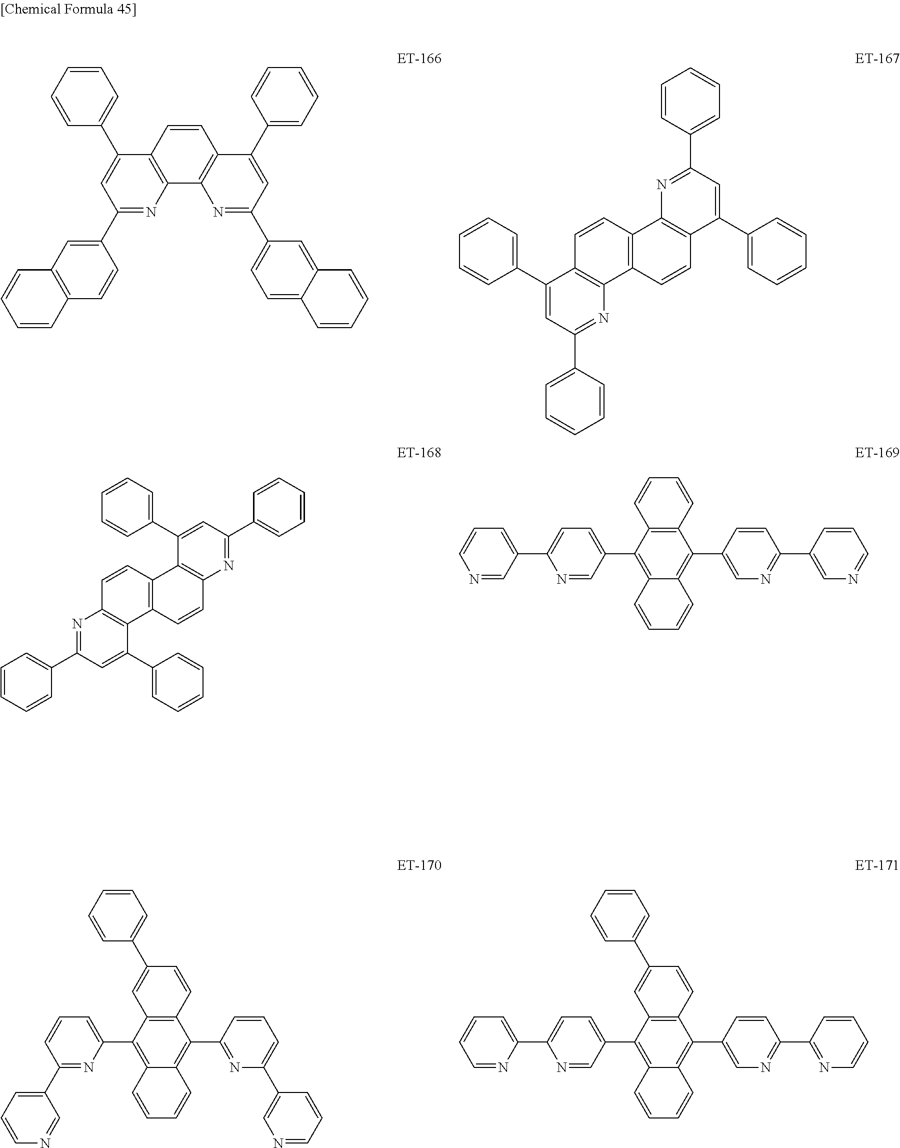 [SMILES: c1ccc(-c2cc(-c3ccc4ccccc4c3)nc3c2ccc2c(-c4ccccc4)cc(-c4ccc5ccccc5c4)nc23)cc1 ; c1ccc(-c2cc(-c3ccccc3)c3ccc4c(ccc5c(-c6ccccc6)cc(-c6ccccc6)nc54)c3n2)cc1 ; c1ccc(-c2cc(-c3ccccc3)c3c(ccc4c3ccc3nc(-c5ccccc5)cc(-c5ccccc5)c34)n2)cc1 ; c1cncc(-c2ccc(-c3c4ccccc4c(-c4ccc(-c5cccnc5)nc4)c4ccccc34)cn2)c1 ; c1ccc(-c2ccc3c(-c4cccc(-c5cccnc5)n4)c4ccccc4c(-c4cccc(-c5cccnc5)n4)c3c2)cc1 ; c1ccc(-c2ccc3c(-c4ccc(-c5ccccn5)nc4)c4ccccc4c(-c4ccc(-c5ccccn5)nc4)c3c2)cc1]

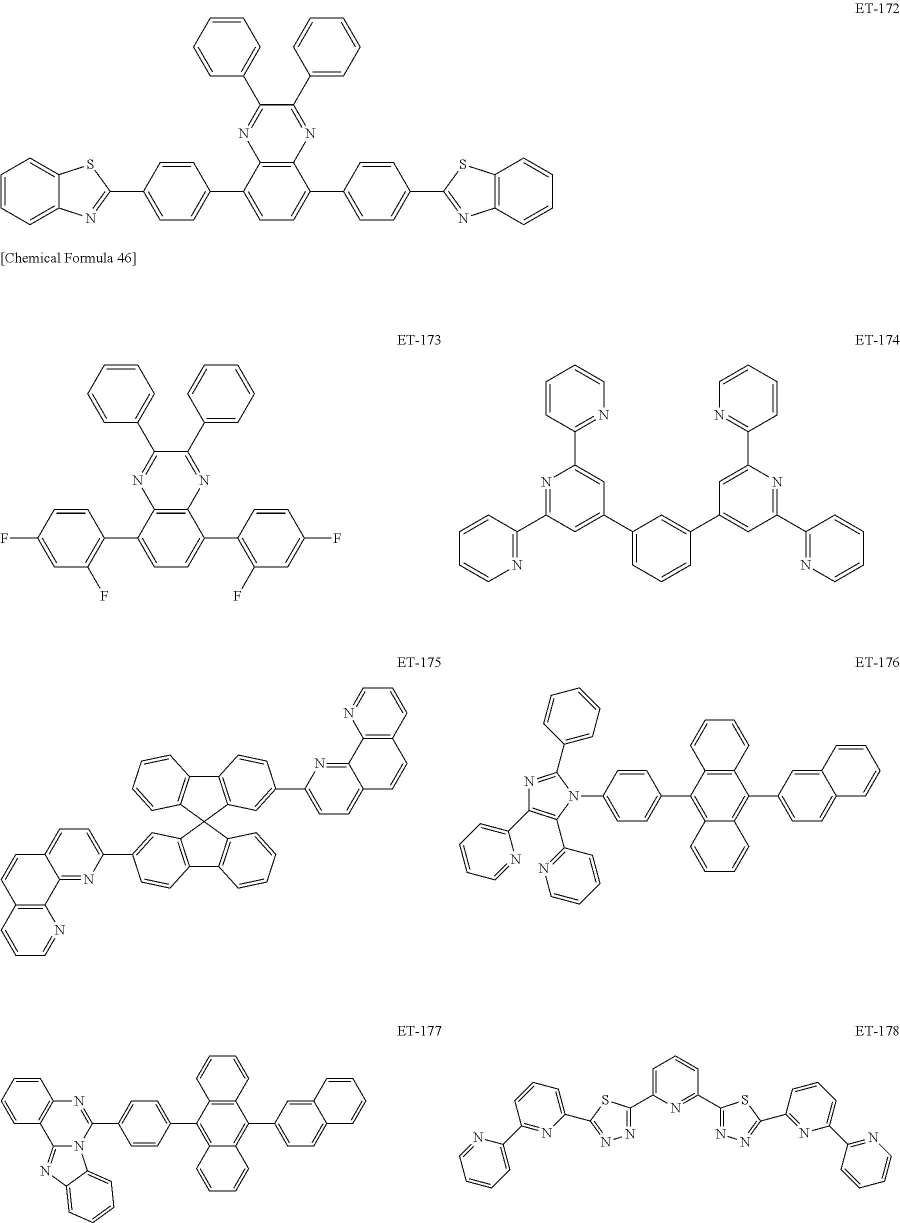

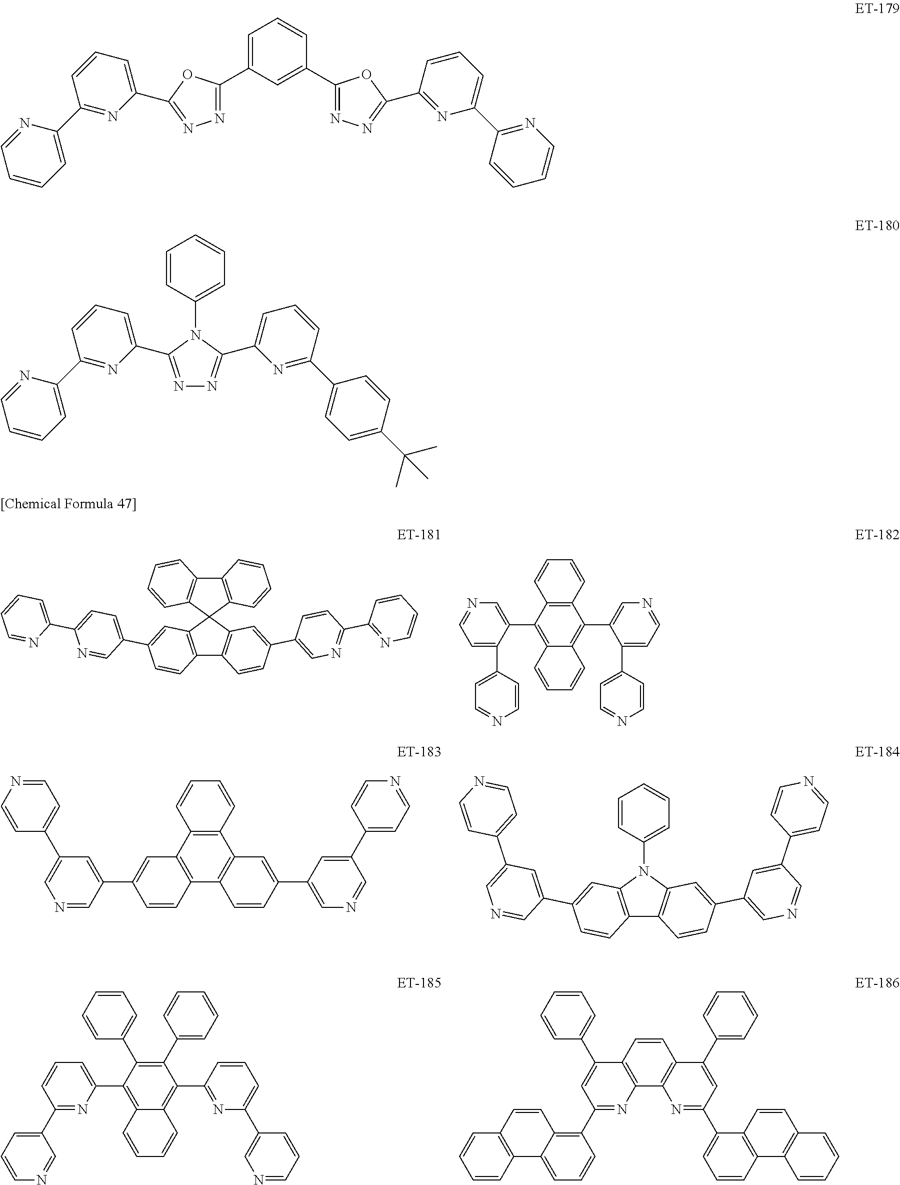

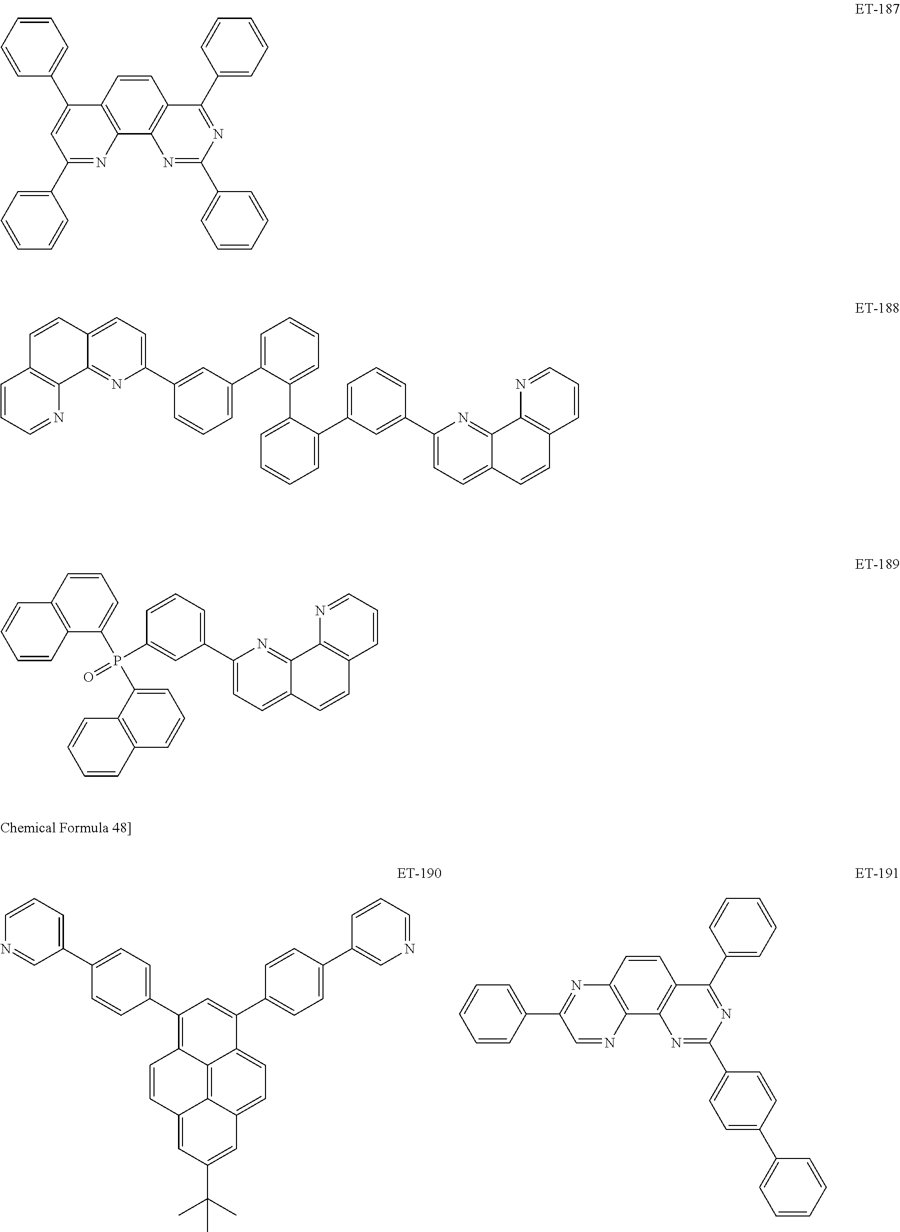 [SMILES: c1ccc(-c2cc(-c3ccccc3)c3ccc4c(-c5ccccc5)nc(-c5ccccc5)nc4c3n2)cc1 ; c1cc(-c2ccc3ccc4cccnc4c3n2)cc(-c2ccccc2-c2ccccc2-c2cccc(-c3ccc4ccc5cccnc5c4n3)c2)c1 ; O=P(c1cccc(-c2ccc3ccc4cccnc4c3n2)c1)(c1cccc2ccccc12)c1cccc2ccccc12 ; CC(C)(C)c1cc2ccc3c(-c4ccc(-c5cccnc5)cc4)cc(-c4ccc(-c5cccnc5)cc4)c4ccc(c1)c2c34 ; c1ccc(-c2ccc(-c3nc(-c4ccccc4)c4ccc5nc(-c6ccccc6)cnc5c4n3)cc2)cc1]

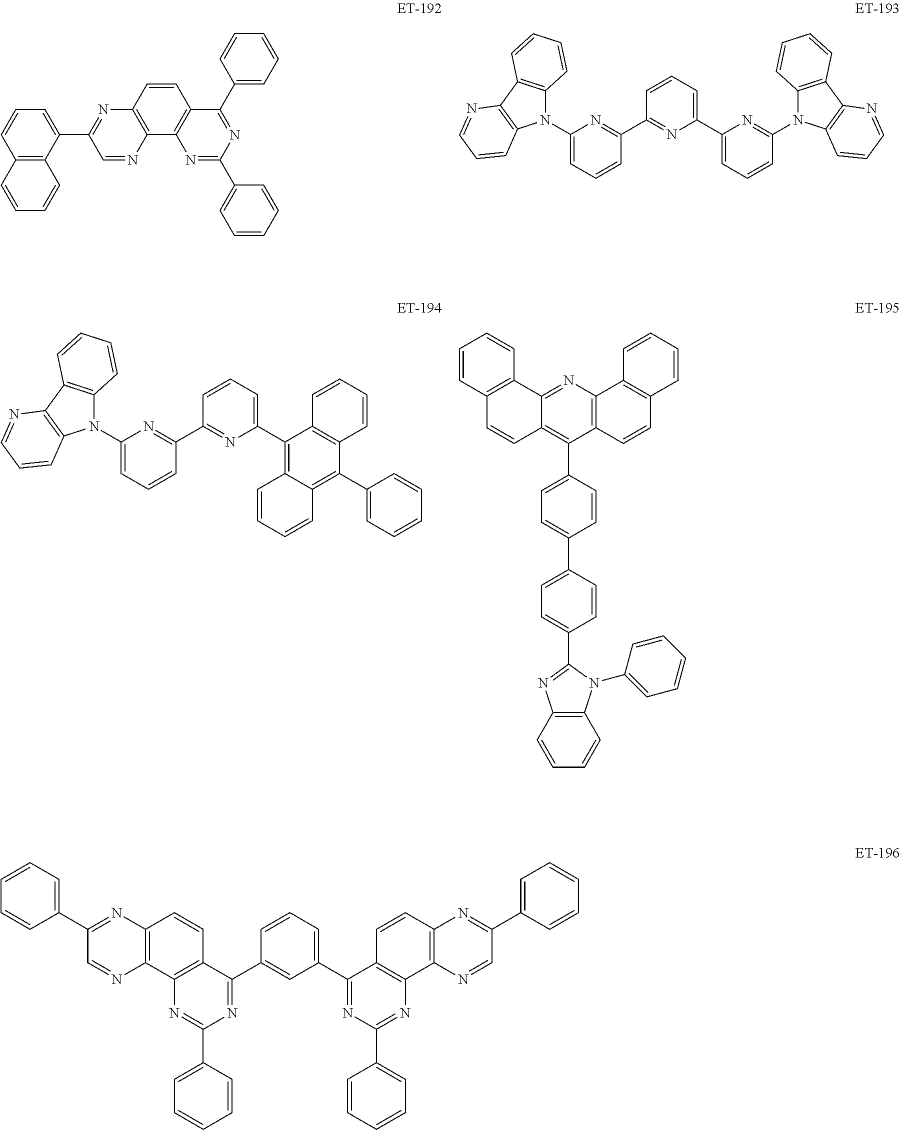 [SMILES: c1ccc(-c2nc(-c3ccccc3)c3ccc4nc(-c5cccc6ccccc56)cnc4c3n2)cc1 ; c1cc(-c2cccc(-n3c4ccccc4c4ncccc43)n2)nc(-c2cccc(-n3c4ccccc4c4ncccc43)n2)c1 ; c1ccc(-c2c3ccccc3c(-c3cccc(-c4cccc(-n5c6ccccc6c6ncccc65)n4)n3)c3ccccc23)cc1 ; c1ccc(-n2c(-c3ccc(-c4ccc(-c5c6ccc7ccccc7c6nc6c5ccc5ccccc56)cc4)cc3)nc3ccccc32)cc1 ; c1ccc(-c2cnc3c(ccc4c(-c5cccc(-c6nc(-c7ccccc7)nc7c6ccc6nc(-c8ccccc8)cnc67)c5)nc(-c5ccccc5)nc43)n2)cc1]

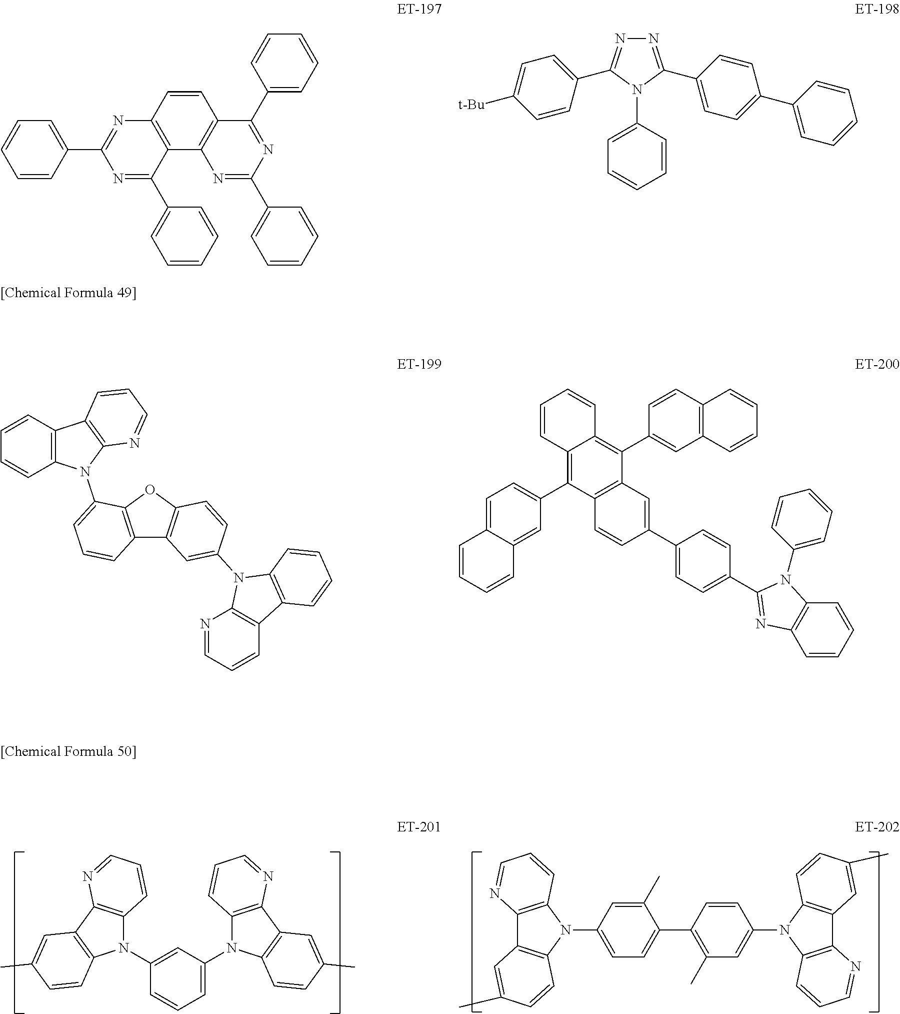

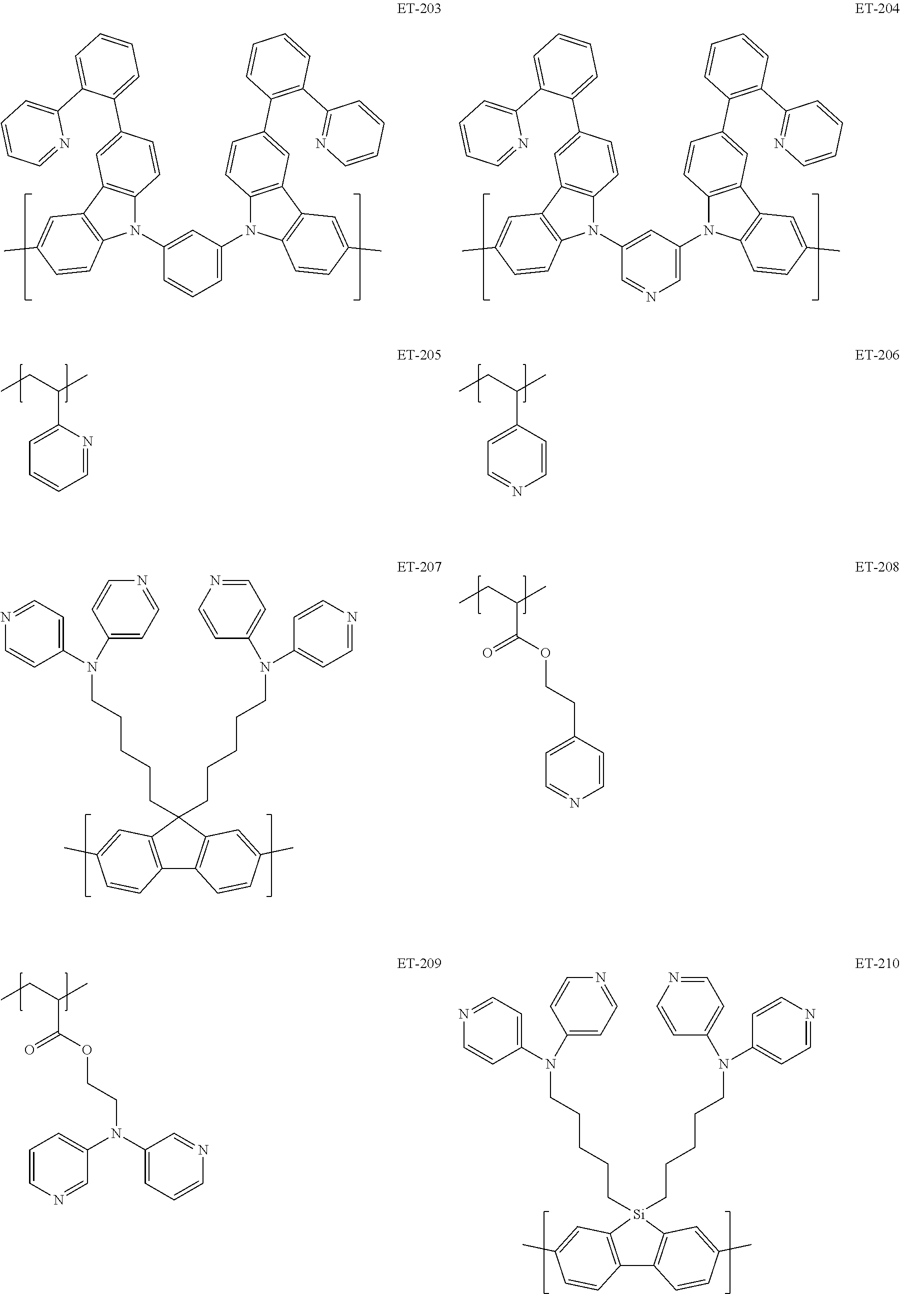

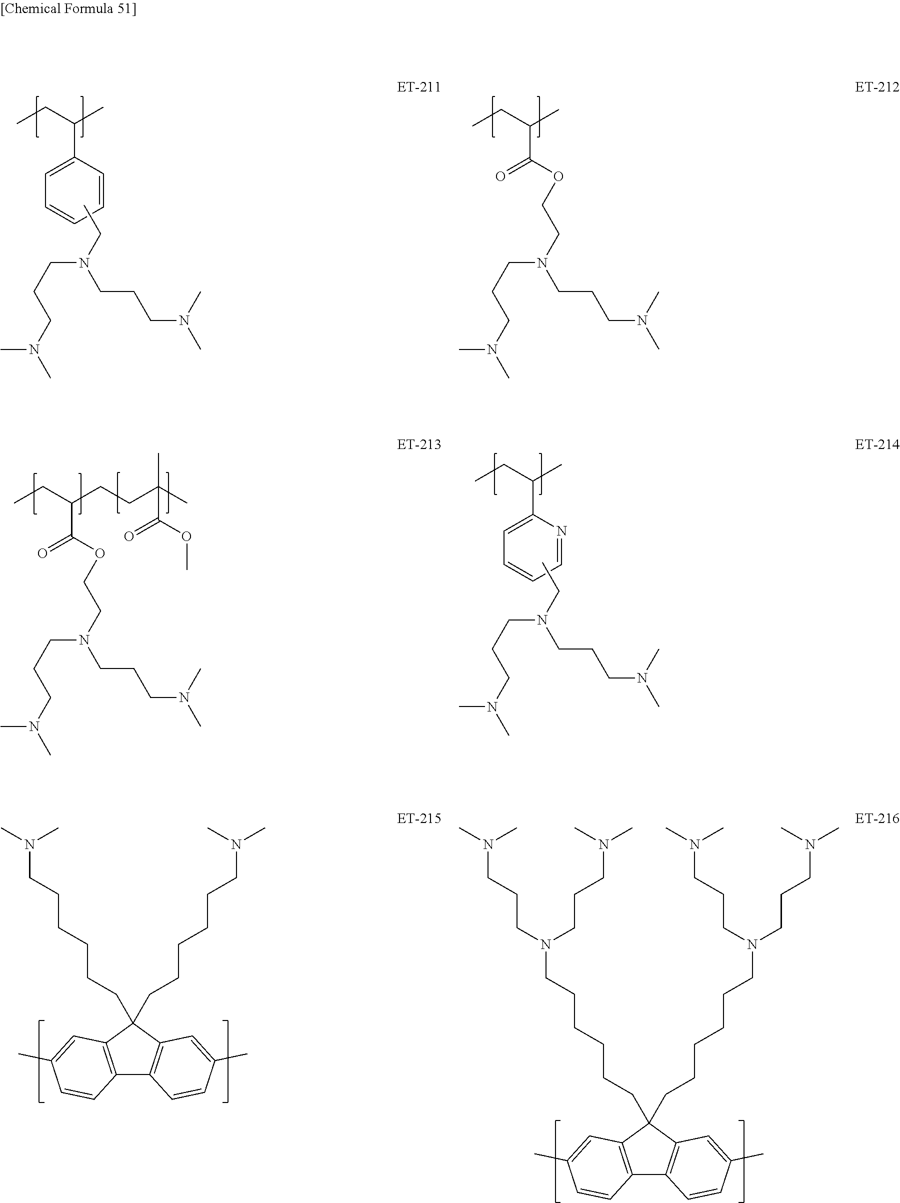

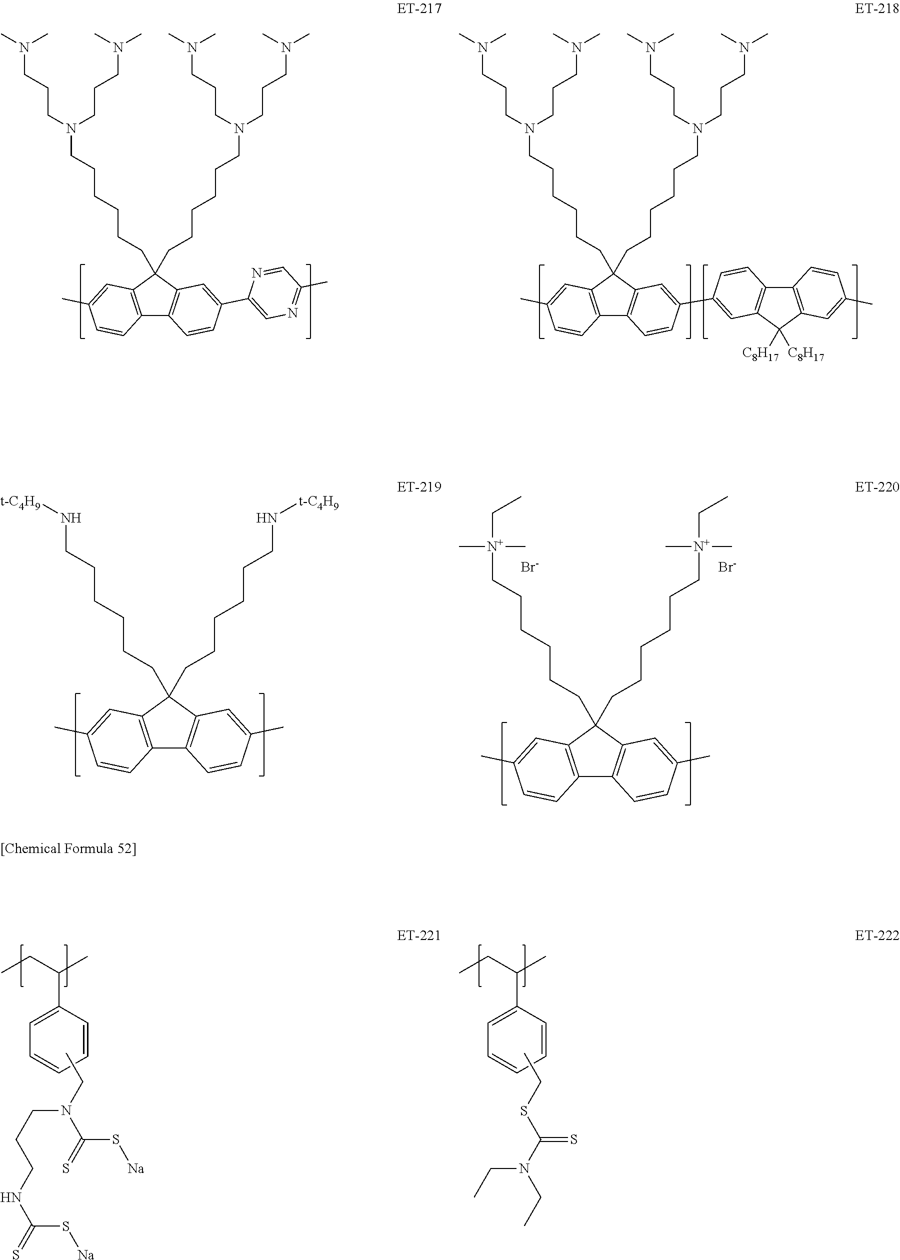

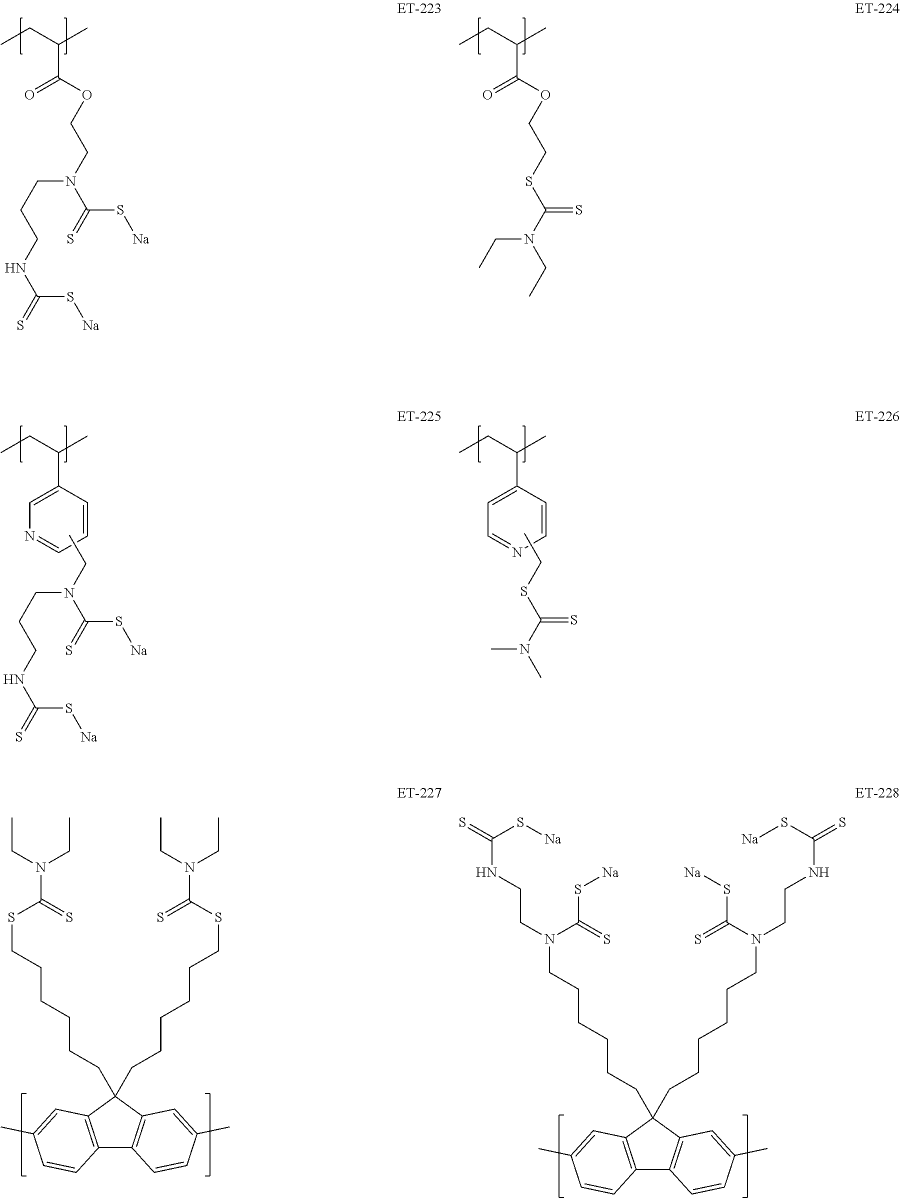

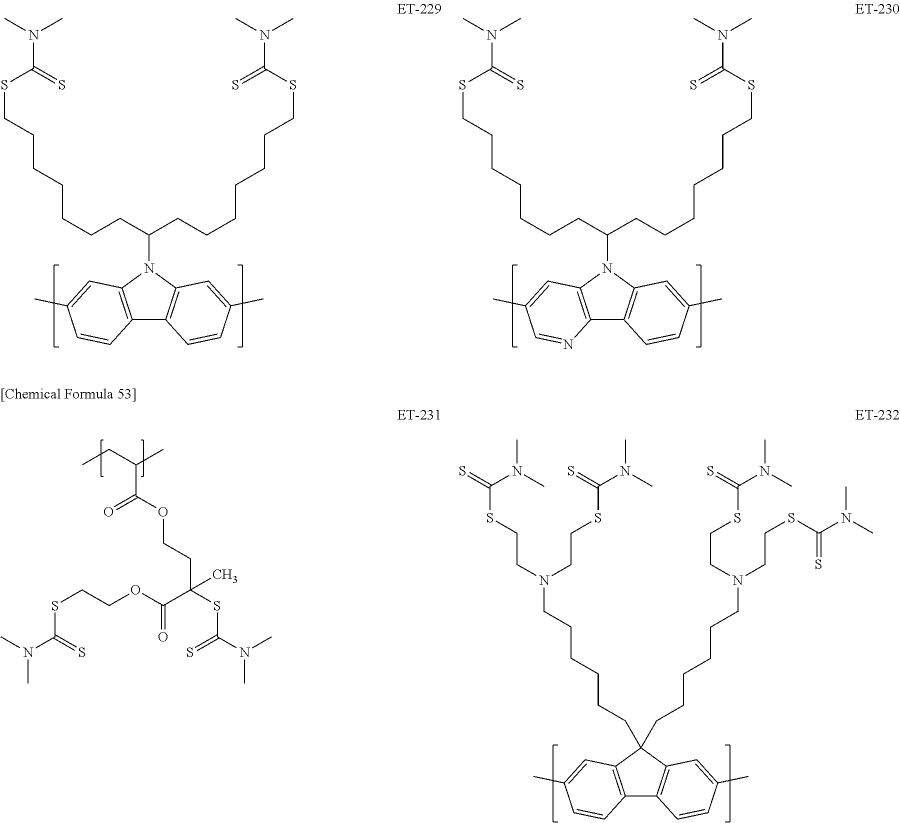

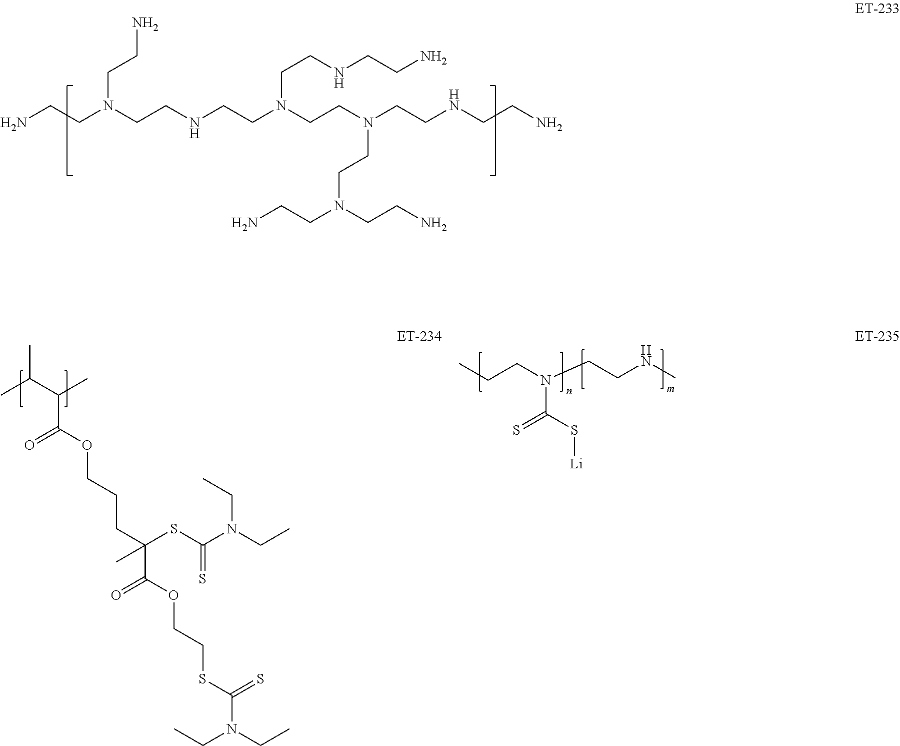

[Synthesis Example of Organic Compound Having Nitrogen Atom]

Synthesis Examples for some of the above exemplified compounds are presented.

(Synthesis of ET-10)

ET-10 was synthesized with reference to JP 2010-235575 A.

(Synthesis of ET-113)

ET-113 was synthesized with reference to JP 2008-222687 A.

(Synthesis of ET-127)

ET-127 was synthesized with reference to JP 2008-69122 A.

(Synthesis of ET-132)

ET-132 was synthesized with reference to JP 2003-336043 A.

(Synthesis of ET-167)

ET-167 was synthesized with reference to WO 2012/082593 A.

(Synthesis of ET-184)

ET-184 was synthesized with reference to JP 2008-247895 A.

(Synthesis of ET-175)

ET-175 was synthesized with reference to JP 2003-59669 A.

(Synthesis of ET-193)

ET-193 was synthesized with reference to WO 2008/020611 A.

(Synthesis of ET-199)

ET-199 was synthesized with reference to WO 2011/004639 A.

(Synthesis of ET-201)

ET-201 was synthesized with reference to JP 2012-104536 A.

(Synthesis of ET-22)

ET-22 was synthesized according to the following Synthetic Formula.

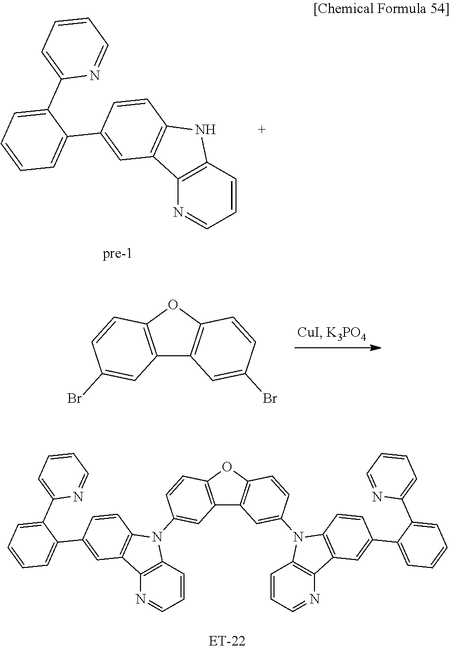 [SMILES: c1ccc(-c2ccccc2-c2ccc3[nH]c4cccnc4c3c2)nc1 ; Brc1ccc2oc3ccc(Br)cc3c2c1 ; c1ccc(-c2ccccc2-c2ccc3c(c2)c2ncccc2n3-c2ccc3oc4ccc(-n5c6ccc(-c7ccccc7-c7ccccn7)cc6c6ncccc65)cc4c3c2)nc1]

First, a solution was prepared by mixing 2,8-dibromodibenzofuran (0.46 g, 1.4 mmol) manufactured by Sigma-Aldrich Co., LLC., a precursor (pre-1: 0.90 g, 2.8 mmol) of ET-22, 15 ml of dimethyl sulfoxide (DMSO), and potassium phosphate (0.89 g, 4.2 mmol) under a nitrogen stream, and this solution was stirred for 10 minutes.

Incidentally, as the pre-1, one synthesized with reference to JP 2010-235575 A was used.

Next, CuI (53 mg, 0.28 mmol) and 6-methylpicolinic acid (0.56 mmol) were mixed with the stirred solution, and the mixed solution was heated at 125° C. for 7 hours. Thereafter, the solution was cooled with water, and while cooling with water, 5 ml of water was added thereto and the solution was stirred for 1 hour.

Subsequently, the crude product precipitated in the solution was filtered, further purified through a column with a mixed solution of heptane:toluene=4:1 to 1:1, and recrystallized in o-dichlorobenzene/acetonitrile, thereby obtaining 0.80 g (yield: 71%) of ET-22.

(Synthesis of ET-124)

ET-124 was synthesized according to the following Synthetic Formula.

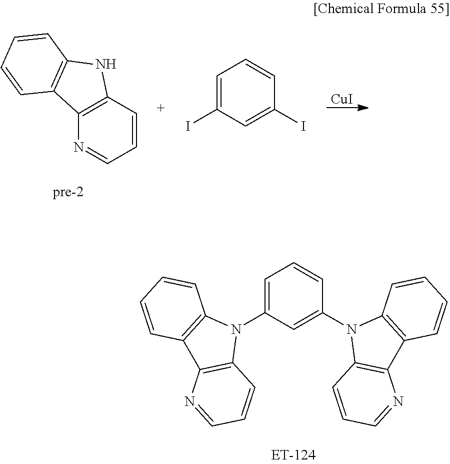

ET-124 was synthesized with reference to JP 2010-235575 A.

Under a nitrogen stream 1,3-diiodobenzene (460 mg, 1.4 mmol) manufactured by Sigma-Aldrich Co., LLC., a precursor (pre-2: 470 mg, 2.8 mmol) of ET-124, 15 ml of DMSO, and potassium phosphate (0.89 g, 4.2 mmol) were mixed, and the mixture was stirred for 10 minutes. CuI (53 mg, 0.28 mmol) and 6-methylpicolinic acid (0.56 mmol) were added thereto, and the mixed solution was heated at 125° C. for 7 hours. While cooling with water, 5 ml of water was added thereto, and the solution was stirred for 1 hour. The crude product precipitated in the solution was filtered, further purified through a column. The resultant product was recrystallized in o-dichlorobenzene/acetonitrile, thereby obtaining 470 mg (yield: 82%) of ET-124.

(Synthesis of ET-144)

ET-144 was synthesized according to the following Synthetic Formula.

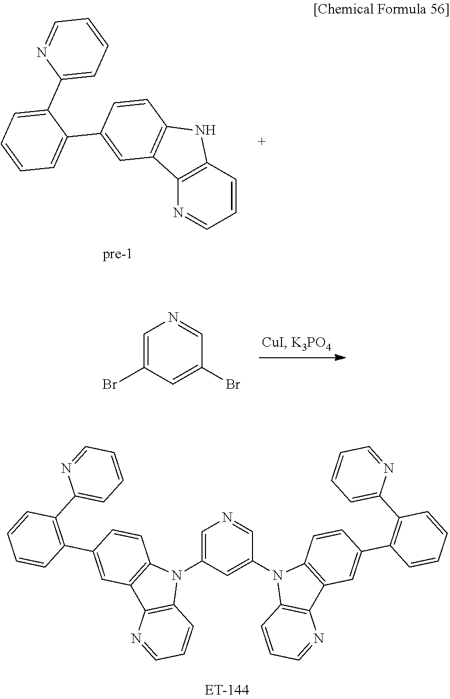 [SMILES: c1ccc(-c2ccccc2-c2ccc3[nH]c4cccnc4c3c2)nc1 ; Brc1cncc(Br)c1 ; c1ccc(-c2ccccc2-c2ccc3c(c2)c2ncccc2n3-c2cncc(-n3c4ccc(-c5ccccc5-c5ccccn5)cc4c4ncccc43)c2)nc1]

ET-144 was synthesized with reference to JP 2010-235575 A.

Under a nitrogen stream, 3,5-dibromopyridine (0.33 g, 1.4 mmol) manufactured by Sigma-Aldrich Co., LLC., a precursor (pre-1: 0.90 g, 2.8 mmol) of ET-144, 15 ml of DMSO, and potassium phosphate (0.89 g, 4.2 mmol) were mixed, and the mixture was stirred for 10 minutes. CuI (53 mg, 0.28 mmol) and 6-methylpicolinic acid (0.56 mmol) were added thereto, and the mixed solution was heated at about 125° C. for 7 hours. While cooling with water, 5 ml of water was added thereto, and the solution was stirred for 1 hour. The crude product precipitated in the solution was filtered, further purified through a column. The resultant product was recrystallized in o-dichlorobenzene/acetonitrile, thereby obtaining 0.75 g (yield: 75%) of ET-144.

(Synthesis of ET-216)

First, a precursor (pre-3) of ET-216 was synthesized with reference to Adv. Mater., VOL.19 (2007), p 2010. The weight average molecular weight of the pre-3 was 4400.

Next, ET-216 was synthesized according to the following Synthetic Formula.

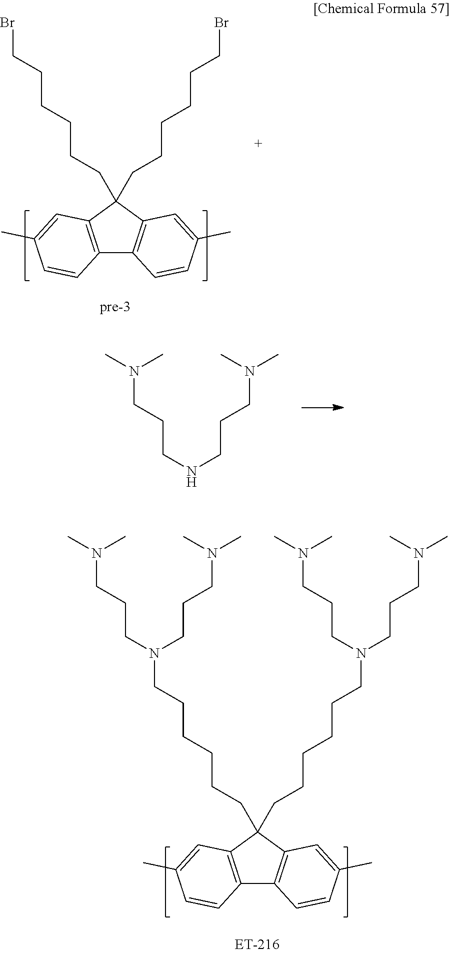

First, a solution was prepared by dissolving the pre-3 (1.0 g) and 3,3′-iminobis(N,N-dimethylpropylamine) (9.0 g manufactured by Sigma-Aldrich Co., LLC.) in a mixed solvent of tetrahydrofuran of 100 ml and N,N-dimethylformamide of 100 ml. The prepared solution was stirred at room temperature (25° C.) for 48 hours to conduct the reaction.

After the reaction was completed, the solvent was distilled off under reduced pressure, and the reprecipitation of the resultant was further conducted in water, thereby obtaining 1.3 g (yield: 90%) of ET-216.

The structure of the compound thus obtained was identified by ¹H-NMR, and the results are presented below. 7.6 to 8.0 ppm (br), 2.88 ppm (br), 2.18 ppm (m), 2.08 ppm (s), 1.50 ppm (m), and 1.05 ppm (br). From this result, it has been confirmed that the compound thus obtained is ET-216.

[Compound Concurrently Usable with Organic Compound Having Nitrogen Atom]

A compound that is used in the electron transport layer and known in the prior art may be concurrently used with the organic compound having a nitrogen atom described above.

As the material that may be concurrently used in the electron transport layer with the organic compound having a nitrogen atom, it is possible to use a compound which exhibits any of electron injection or transport properties and hole barrier properties.

Examples thereof may include a nitrogen-containing aromatic heterocyclic derivative (a carbazole derivative, an azacarbazole derivative (one obtained by substituting one or more of the carbon atoms constituting the carbazole ring with a nitrogen atom), a pyridine derivative, a pyrimidine derivative, a pyrazine derivative, a pyridazine derivative, a triazine derivative, a quinoline derivative, a quinoxaline derivative, a phenanthroline derivative, an azatriphenylene derivative, an oxazole derivative, a thiazole derivative, an oxadiazole derivative, a thiadiazole derivative, a triazole derivative, a benzimidazole derivative, a benzoxazole derivative, a benzothiazole derivatives, or the like), a dibenzofuran derivative, a dibenzothiophene derivative, a silole derivative, and an aromatic hydrocarbon ring derivative (a naphthalene derivative, an anthracene derivative, a triphenylene, or the like).

In addition, a metal complex having a quinolinol backbone or dibenzo-quinolinol backbone as a ligand, for example, tris(8-quinolinol)aluminum (Alq), tris(5,7-dichloro-8-quinolinol)aluminum, tris(5,7-dibromo-8-quinolinol)aluminum, tris(2-methyl-8-quinolinol)aluminum, tris(5-methyl-8-quinolinol)aluminum, or bis(8-quinolinol)zinc (Znq) and a metal complex obtained by substituting the central metal of these metal complexes with In, Mg, Cu, Ca, Sn, Ga or Pb can also be concurrently used as an electron transporting material.

In addition to these, metal-free or metal phthalocyanine or those obtained by substituting the end of them with an alkyl group or a sulfonic acid group is also preferably concurrently used as an electron transporting material. In addition, a distyryl pyrazine derivative exemplified as a material for the luminous layer can also be concurrently used as an electron transporting material, and an inorganic semiconductor such as n-type Si or n-type SiC can also be concurrently used as an electron transporting material in the same manner as in the hole injection layer and the hole transport layer.

In addition, it is also possible to concurrently use a polymer material obtained by introducing these materials into a chain of the polymer or by using these materials as a main chain of the polymer.

In the electron transport layer according to the present invention, it is preferable that the organic compound having a nitrogen atom contains an electron donating dopant.

In other words, it is preferable to form the electron transport layer exhibiting high n-properties (electron-rich) by doping the electron transport layer with a doping material as a guest material. This is because it is possible to enhance the conductivity of the electron transport layer and to obtain an electron transport layer having a thicker layer thickness when an electron donating dopant is contained.

Examples of the n-type dopant material may include an n-type dopant such as an alkali metal such as lithium or cesium, an alkaline earth metal such as magnesium or calcium, a metal complex described in J. Am. Chem. Soc., 2003, 125, 16040 or JP 2007-526640 W, and a metal compound such as lithium fluoride or cesium carbonate, and an organic substance described in JP 2007-273978 A. Specific examples of the electron transport layer having such a constitution may include those described in the literatures such as JP 4-297076 A, JP 10-270172 A, JP 2000-196140 A, JP 2001-102175 A, and J. Appl. Phys., 2004, 95, 5773.

These n-type dopant materials may be selected depending on the purpose although the driving voltage lowering effect thereof and the durability and process handleability (handling at the time of production, for example, at the time of loading into the vacuum deposition apparatus) are traded off in some cases, and an alkali metal, an alkaline earth metal, and a metal complex are preferable from the viewpoint of lowering the driving voltage.

<<Luminous Layer>>

The luminous layer according to the present invention is a layer that provides a place at which the electron and the hole that are injected from the electrodes or the adjacent layers are rebound to emit light via an exciton, and the part at which light emits may be in the layer of the luminous layer or at the interface between the luminous layer and the adjacent layer. The constitution of the luminous layer according to the present invention is not particularly limited as long as it meets the requirements specified in the present invention.

The sum of the layer thicknesses of the luminous layer is not particularly limited, but it is preferably adjusted to be in a range of from 2 nm to 5 μm, and it is more preferably adjusted to be in a range of from 2 to 500 nm, and it is even more preferably adjusted to be in a range of from 5 to 200 nm from the viewpoint of uniformity of the film to be formed, the prevention of application of an unrequired high voltage at the time of emitting light, and the improvement in stability of the luminous color with respect to the driving current.

In addition, the layer thickness of the individual luminous layers of the present invention is preferably adjusted to be in a range of from 2 nm to 1 μm, and it is more preferably adjusted to be in a range of from 2 to 200 nm, and it is even more preferably adjusted to be in a range of from 3 to 150 nm.

It is preferable that the luminous layer of the present invention contains a luminescent dopant (also referred to as a luminescent dopant compound, a dopant compound, or simply a dopant) and a host compound (referred to as a matrix material, a luminescent host compound, or simply a host).

(1) Luminescent Dopant

The luminescent dopant used in the present invention will be described.

As the luminescent dopant, a fluorescence emitting dopant (also referred to as a fluorescent dopant or a fluorescent compound) and a phosphorescence emitting dopant (also referred to as a phosphorescent dopant or a phosphorescent compound) are preferably used. In the present invention, it is preferable that at least one layer of the luminous layers contains a phosphorescence emitting dopant.

The concentration of the luminescent dopant in the luminous layer can be arbitrarily determined based on the requirements of the particular dopant and device to be used, and the luminescent dopant may be contained at a uniform concentration in the layer thickness direction of the luminous layer or may have arbitrary concentration distribution.

In addition, as the luminescent dopant used in the present invention, plural kinds may be concurrently used, or a combination of dopants having different structures or a combination of a fluorescence emitting dopant and a phosphorescence emitting dopant may be used. This makes it possible to obtain an arbitrary luminous color.

The color of light emitted by the organic EL element of the present invention or the compound used in the present invention is determined by the color at the time of applying the results measured using a spectroradiometer CS-1000 (manufactured by Konica Minolta, Inc.) to the CIE chromaticity coordinate in FIG. 4.16 on page 108 of the “New Version Color Science Handbook” (edited by Color Science Association of Japan, University of Tokyo Press, 1985).

In the present invention, it is also preferable that one layer or plural layers of the luminous layers contain plural luminescent dopants exhibiting different luminous colors so as to emit white light.

The combination of the luminescent dopants exhibiting white is not particularly limited, but examples thereof may include a combination of blue and orange or a combination of blue, green, and red.

It is preferable that the white in the organic EL element of the present invention has a chromaticity in a region of x=0.39±0.09 and y=0.38±0.08 in the CIE1931 color system at 1000 cd/m² when 2-degree viewing angle front brightness is measured by the method described above.

(1.1) Phosphorescence Emitting Dopant

The phosphorescence emitting dopant (hereinafter, also referred to as the “phosphorescent dopant”) used in the present invention will be described.

The phosphorescent dopant used in the present invention is a compound from which luminescence from the excited triplet state is observed, and specifically it is a compound that exhibits phosphorescence at room temperature (25° C.). It is defined as a compound which has a phosphorescence quantum yield of 0.01 or more at 25° C., but a preferred phosphorescence quantum yield is 0.1 or more.

The phosphorescence quantum yield can be measured by the method described in the Experimental Chemistry 7, Fourth Edition, Spectroscopy II, page 398 (1992, published by Maruzen). The phosphorescence quantum yield in a solution can be measured using various solvents, but the phosphorescent dopant used in the present invention is only desired to achieve the phosphorescence quantum yield (0.01 or more) described above in any of arbitrary solvents.

The luminescence of the phosphorescent dopant is divided into two types in principle, and one is an energy transfer type in which the recombination of carriers occurs on the host compound to which the carrier is transported so as to generate the excited state of the host compound, and luminescence from the phosphorescent dopant is obtained by transferring this energy to the phosphorescent dopant. The other one is a carrier trap type in which the phosphorescent dopant is a carrier trap and the recombination of carriers occurs on the phosphorescent dopant so as to obtain luminescence from the phosphorescent dopant. In either case, it is a condition that the energy of the phosphorescent dopant in the excited state is lower than the energy of the host compound in the excited state.

Specific examples of known phosphorescent dopant which can be used in the present invention may include the compounds that are described in the following literatures.

Nature, 395, 151 (1998), Appl. Phys. Lett., 78, 1622 (2001), Adv. Mater., 19, 739 (2007), Chem. Mater., 17, 3532 (2005), Adv. Mater., 17, 1059 (2005), WO 2009/100991 A, WO 2008/101842 A, WO 2003/040257 A, US 2006/835,469, US 2006/0,202,194, US 2007/0,087,321, US 2005/0,244,673, Inorg. Chem., 40, 1704 (2001), Chem. Mater., 16, 2480 (2004), Adv. Mater., 16, 2003 (2004), Angew. Chem. Int. Ed., 2006, 45, 7800, Appl. Phys. Lett., 86, 153505 (2005), Chem. Lett., 34, 592 (2005), Chem. Commun., 2906 (2005), Inorg. Chem., 42, 1248 (2003), WO 2009/050290 A, WO 2002/015645 A, WO 2009/000673 A, US 2002/0,034,656, U.S. Pat. No. 7,332,232, US 2009/0,108,737, US 2009/0,039, 776, U.S. Pat. No. 6,921,915, U.S. Pat. No. 6,687,266, US 2007/0,190,359, US 2006/0,008,670, US 2009/0,165,846, US 2008/0,015,355, U.S. Pat. No. 7,250,226, U.S. Pat. No. 7,396,598, US 2006/0,263,635, US 2003/0,138,657, US 2003/0,152,802, U.S. Pat. No. 7,090,928, Angew. Chem. Int. Ed., 47, 1 (2008), Chem. Mater., 18, 5119 (2006), Inorg. Chem., 46, 4308 (2007), Organometallics, 23, 3745 (2004), Appl. Phys. Lett., 74, 1361 (1999), WO 2002/002714 A, WO 2006/009024 A, WO 2006/056418 A, WO 2005/019373 A, WO 2005/123873 A, WO 2005/123873 A, WO 2007/004380 A, WO 2006/082742 A, US 2006/0,251,923, US 2005/0,260,441, U.S. Pat. No. 7,393,599, U.S. Pat. No. 7,534,505, U.S. Pat. No. 7,445,855, US 2007/0,190,359, US 2008/0,297,033, U.S. Pat. No. 7,338,722, US 2002/0,134,984, U.S. Pat. No. 7,279,704, US 2006/098,120, US 2006/103,874, WO 2005/076380 A, WO 2010/032663 A, WO 2008/140115 A, WO 2007/052431 A, WO 2011/134013 A, WO 2011/157339 A, WO 2010/086089 A, WO 2009/113646 A, WO 2012/020327 A, WO 2011/051404 A, WO 2011/004639 A, WO 2011/073149A, US 2012/228,583, US 2012/212,126, JP2012-069737 A, JP 2011-181303, JP 2009-114086 A, JP 2003-81988 A, JP 2002-302671 A, JP 2002-363552 A, and the like.

Among them, examples of the preferred phosphorescent dopant may include an organometallic complex having Ir (iridium) as the central metal. Even more preferably, a complex containing at least one coordination mode of a metal-carbon bond, a metal-nitrogen bond, a metal-oxygen bond, or a metal-sulfur bond is preferable.

(1.2) Fluorescence Emitting Dopant

The fluorescence emitting dopant (hereinafter, also referred to as the “fluorescent dopant”) used in the present invention will be described.

The fluorescent dopant used in the present invention is a compound capable of emitting light from the excited singlet state, and it is not particularly limited as long as luminescence from the excited singlet state is observed.

Examples of the fluorescent dopant used in the present invention may include an anthracene derivative, a pyrene derivative, a chrysene derivative, a fluoranthene derivative, a perylene derivative, a fluorene derivative, an arylacetylene derivative, a styrylarylene derivative, a styrylamine derivative, an arylamine derivative, a boron complex, a coumarin derivative, a pyran derivative, a cyanine derivative, a croconium derivative, a squarylium derivative, an oxobenzanthracene derivative, a fluorescein derivative, a rhodamine derivative, a pyrylium derivative, a perylene derivative, a polythiophene derivative, or a rare earth complex-based compound.

In addition, a luminescent dopant utilizing delayed fluorescence has also been developed in recent years, and these may be used.

Specific examples of the luminescent dopant utilizing delayed fluorescence may include the compounds described in WO 2011/156793 A, JP 2011-213643 A, JP 2010-93181 A, and the like, but the present invention is not limited thereto.

(2) Host Compound

The host compound used in the present invention is a compound that is mainly responsible for the injection and transport of a charge in the luminous layer, and luminescence of the host compound itself is not substantially observed in the organic EL element.

The host compound is preferably a compound which has a phosphorescence quantum yield of phosphorescence at room temperature (25° C.) of less than 0.1, and it is even more preferably a compound which has a phosphorescence quantum yield of less than 0.01. In addition, it is preferable that the mass ratio of the host compound in the luminous layer is 20% or more among the compounds contained in the luminous layer.

In addition, it is preferable that the energy of the host compound in the excited state is higher than the energy of the luminescent dopant contained in the same layer in the excited state.

The host compound may be used singly or plural kinds thereof may be used concurrently. It is possible to adjust the transfer of a charge and to increase the efficiency of the organic EL element by using plural kinds of host compounds.

The host compound which can be used in the present invention is not particularly limited, and it is possible to use a compound that is used in an organic EL element of the prior art. It may be a low molecular weight compound or a polymer compound having a repeating unit, and it may be a compound having a reactive group such as a vinyl group or an epoxy group.

It is preferable that a known host compound has a high glass transition temperature (Tg) from the viewpoint of preventing luminescence from shifting to a longer wavelength while having hole transporting ability or electron transporting ability and further of stably operating the organic EL element against heat generation at the time of driving the element at a high temperature or during driving the element. A host compound having a Tg of 90° C. or higher is preferable, and the Tg is more preferably 120° C. or higher.

Here, the glass transition point (Tg) is a value determined by a method conforming to JIS K 7121-2012 using differential scanning calorimetry (DSC).

Specific examples of the known host compound that is used in the organic EL element of the present invention may include the compounds described in the following literatures, but the present invention is not limited thereto.

JP 2001-257076 A, JP 2002-308855 A, JP 2001-313179 A, JP 2002-319491 A, JP 2001-357977 A, JP 2002-334786 A, JP 2002-8860 A, JP 2002-334787 A, JP 2002-15871 A, JP 2002-334788 A, JP 2002-43056 A, JP 2002-334789 A, JP 2002-75645 A, JP 2002-338579 A, JP 2002-105445 A, JP 2002-343568 A, JP 2002-141173 A, JP 2002-352957 A, JP 2002-203683 A, JP 2002-363227 A, JP 2002-231453 A, JP 2003-3165 A, JP 2002-234888 A, JP 2003-27048 A, JP 2002-255934 A, JP 2002-260861 A, JP 2002-280183 A, JP 2002-299060 A, JP 2002-302516 A, JP 2002-305083 A, JP 2002-305084 A, JP 2002-308837 A, US 2003/0,175,553, US 2006/0,280,965, US 2005/0,112,407, US 2009/0,017,330, US 2009/0,030,202, US 2005/0,238,919, WO 2001/039234 A, WO 2009/021126 A, WO 2008/056746 A, WO 2004/093207 A, WO 2005/089025 A, WO 2007/063796 A, WO 2007/063754 A, WO 2004/107822 A, WO 2005/030900 A, WO 2006/114966 A, WO 2009/086028 A, WO 2009/003898 A, WO 2012/023947 A, JP 2008-074939 A, JP 2007-254297 A, EP 2034538 B, and the like.

<<Hole Blocking Layer>>

The hole blocking layer is a layer having the function of an electron transport layer in a broad sense, and it is preferably composed of a material which has small ability of transporting a hole while having a function of transporting an electron, and it is possible to increase the probability of recombination of an electron with a hole by blocking a hole while transporting an electron.

In addition, it is possible to use the constitution of the electron transport layer described above as the hole blocking layer if necessary.

It is preferable that the hole blocking layer provided in the organic EL element of the present invention is provided so as to be adjacent to the negative electrode side of the luminous layer.

The layer thickness of the hole blocking layer used in the present invention is preferably in a range of from 3 to 100 nm and even more preferably in a range of from 5 to 30 nm.

As the material used for the hole blocking layer, the material used for the electron transport layer described above is preferably used, and also the material used as the host compound described above is also preferably used in the hole blocking layer.

<<Hole Transport Layer>>

In the present invention, the hole transport layer is only desired to be composed of a material having a function of transporting a hole and to have a function of delivering the hole injected from the positive electrode to the luminous layer.

The total layer thickness of the hole transport layer used in the present invention is not particularly limited, but it is usually in a range of from 5 nm to 5 μm, more preferably from 2 to 500 nm, and even more preferably from 5 to 200 nm.

The material (hereinafter, referred to as the hole transporting material) used for the hole transport layer is only desired to have any of hole injection or transport properties or electron barrier properties, and it is possible to select and use an arbitrary one among the compounds known in the prior art.

Examples thereof may include a porphyrin derivative, a phthalocyanine derivative, an oxazole derivative, an oxadiazole derivative, a triazole derivative, an imidazole derivative, a pyrazoline derivative, a pyrazolone derivative, a phenylenediamine derivative, a hydrazone derivative, a stilbene derivative, a polyarylalkane derivative, a triarylamine derivative, a carbazole derivative, an indolocarbazole derivative, an isoindole derivative, an acene-based derivative such as anthracene or naphthalene, a fluorene derivative, a fluorenone derivative, polyvinyl carbazole, a polymer material or oligomer obtained by introducing an aromatic amine into the main chain or a side chain, a polysilane, and a conductive polymer or oligomer (for example, PEDOT/PSS, an aniline-based copolymer, polyaniline, and polythiophene, or the like).

Examples of the triarylamine derivative may include a benzidine type represented by α-NPD, a starburst type represented by MTDATA, and a compound having a fluorene or anthracene at the triarylamine connecting core portion.

In addition, it is also possible to use a hexaazatriphenylene derivative as described in JP 2003-519432 W or JP 2006-135145 A as a hole transporting material in the same manner.

Furthermore, it is also possible to use a hole transport layer that is doped with impurities and exhibits high p-properties. Examples thereof may include those described in JP 4-297076A, JP 2000-196140 A, JP 2001-102175 A, J. Appl. Phys. 95, 5773 (2004), and the like.

In addition, it is also possible to use the so-called p-type hole transporting material as described in JP 11-251067 A and a literature written by J. Huang et al. (Applied Physics Letters 80 (2002), p. 139) or an inorganic compound such as p-type Si or p-type SiC. Furthermore, an ortho-metalated organometallic complex having Ir or Pt as the central metal as represented by Ir(ppy)₃ is also preferably used.

It is possible to use those described above as the hole transporting material, but a polymer material or oligomer obtained by introducing a triarylamine derivative, a carbazole derivative, an indolocarbazole derivative, an azatriphenylene derivative, an organometallic complex, or an aromatic amine in the main chain or a side chain is preferably used.

Specific examples of the known preferred hole transporting material that is used in the organic EL element of the present invention may include the compounds described in the following literatures in addition to the literatures listed above, but the present invention is not limited thereto.

For example, Appl. Phys. Lett., 69, 2160 (1996), J. Lumin., 72-74, 985 (1997), Appl. Phys. Lett., 78, 673 (2001), Appl. Phys. Lett., 90, 183503 (2007), Appl. Phys. Lett., 90, 183503 (2007), Appl. Phys. Lett., 51, 913 (1987), Synth. Met., 87, 171 (1997), Synth. Met., 91, 209 (1997), Synth. Met., 111, 421 (2000), SID Symposium Digest, 37, 923 (2006), J. Mater. Chem., 3, 319 (1993), Adv. Mater., 6, 677 (1994), Chem. Mater., 15, 3148 (2003), US 2003/0,162,053, US 2002/0,158,242, US 2006/0,240,279, US 2008/0,220,265, U.S. Pat. No. 5,061,569, WO 2007/002683A, WO2009/018009A, EP 650955 B, US 2008/0,124,572, US 2007/0,278,938, US 2008/0,106,190, US 2008/0,018,221, WO 2012/115034 A, JP 2003-519432 W, JP 2006-135145 A, U.S. Ser. No. 13/585,981, and the like.

The hole transporting material may be used singly, or plural kinds thereof may be used concurrently.

<<Electron Blocking Layer>>

The electron blocking layer is a layer having the function of a hole transport layer in a broad sense, and it is preferably composed of a material which has small ability of transporting an electron while having a function of transporting a hole, and it is possible to increase the probability of recombination of an electron with a hole by blocking an electron while transporting a hole.

In addition, it is possible to use the constitution of the hole transport layer described above as the electron blocking layer if necessary.

It is preferable that the electron blocking layer provided in the organic EL element of the present invention is provided so as to be adjacent to the positive electrode side of the luminous layer.

The layer thickness of the electron blocking layer used in the present invention is preferably in a range of from 3 to 100 nm and even more preferably in a range of from 5 to 30 nm.

As the material used for the electron blocking layer, the material used for the hole transport layer described above is preferably used, and also the material used as the host compound described above is also preferably used in the electron blocking layer.

<<Hole Injection Layer>>

The hole injection layer (also referred to as the “positive electrode buffer layer”) used in the present invention is a layer that is provided between the positive electrode and the luminous layer in order to lower the driving voltage and to improve the brightness, and it is described in detail in the “Organic EL Element and its Industrialization Front (Nov. 30, 1998 published by (C) NTS, Inc.)”, Part II, Chapter 2 “Electrode Material” (pp. 123-166).

In the present invention, the hole injection layer is provided if necessary, and it may be present between the positive electrode and the luminous layer as described above or between the positive electrode and the hole transport layer.

The hole injection layer is described in detail in JP 9-45479 A, JP 9-260062 A, JP 8-288069 A, and the like as well, and examples of the material used for the hole injection layer may include the materials used for the hole transport layer described above.

Among them, a phthalocyanine derivative represented by copper phthalocyanine, a hexaazatriphenylene derivative as described in JP 2003-519432 W, JP 2006-135145 A, or the like, a metal oxide represented by vanadium oxide, a conductive polymer such as amorphous carbon, polyaniline (emeraldine), or polythiophene, an ortho-metalated complex represented by tris(2-phenylpyridine)iridium complex, a triarylamine derivative, and the like are preferable.

The materials used for the hole injection layer may be used singly, or plural kinds thereof may be used concurrently.

<<Other Additives>>

The organic layer that is used in the present invention and described above may further contain other additives.

Examples of the additive may include a halogen such as bromine, iodine, or chlorine or a halogenated compound, an alkali metal or an alkaline earth metal such as Pd, Ca, or Na, a compound or complex of a transition metal, a salt, and the like.

The content of the additive can be arbitrarily determined, but it is preferably 1000 ppm or less, more preferably 500 ppm or less, and even more preferably is 50 ppm or less with respect to the total mass % of the layer which contains the additive.

Provided that, it is not in this range depending on the purpose that the transportability of an electron or a hole is improved, the energy transfer of the exciton is facilitated, or the like.

<<Method for Forming Organic Layer>>

The method for forming the organic layer (the hole injection layer, the hole transport layer, the luminous layer, the hole blocking layer, the electron transport layer, the electron injection layer, or the like) used in the present invention will be described.

The method for forming the organic layer is not particularly limited, and it is possible to use a forming method known in the prior art by a vacuum deposition method, a wet method (also referred to as the wet process.), or the like.

As the wet method, there are a spin coating method, a casting method, an ink-jet method, a printing method, a die coating method, a blade coating method, a roll coating method, a spray coating method, a curtain coating method, an LB method (Langmuir-Blodgett method), and the like, but a method that is highly suitable for the roll-to-roll method, such as a die coating method, a roll coating method, an ink-jet method, or a spray coating method is preferable from the viewpoint of being likely to obtain a uniform thin film and having high productivity.

As a liquid medium for dissolving or dispersing the materials used in the organic EL element of the present invention, it is possible to use, for example, a ketone such as methyl ethyl ketone or cyclohexanone, a fatty acid ester such as ethyl acetate, a halogenated hydrocarbon such as dichlorobenzene, an aromatic hydrocarbon such as toluene, xylene, mesitylene, or cyclohexylbenzene, an aliphatic hydrocarbon such as cyclohexane, decalin, or dodecane, and an organic solvent such as N,N-dimethylformamide (DMF) or DMSO.

In addition, as the dispersing method, it is possible to disperse the materials by a dispersing method such as ultrasonic waves, high shear dispersion, or media dispersion.

Furthermore, different film forming methods may be applied for each layer. In the case of employing a vapor deposition method for film formation, the vapor deposition conditions vary depending on the kinds of the compounds to be used, but in general, it is desirable to appropriately select in a range in which the boat heating temperature is from 50 to 450° C., the vacuum degree is from 1×10⁻⁶ to 1×10⁻² Pa, the deposition rate is from 0.01 to 50 nm/sec, the substrate temperature is from −50 to 300° C., the layer thickness is from 0.1 nm to 5 μm and preferably from 5 to 200 nm.

For the formation of the organic layer used in the present invention, it is preferable to consistently fabricate from the hole injection layer to the negative electrode by vacuum drawing of one time, but the transparent substrate may be taken out in the middle of the fabrication so as to be subjected to a different film forming method. In that case, it is preferable to conduct the operation in a dry inert gas atmosphere.

<<Positive Electrode>>

As the positive electrode (hereinafter, also referred to as the anode), those are preferably used which contain a metal, an alloy, an electrically conductive compound, or a mixture thereof which has a great work function (4 eV or more, preferably 4.5 eV or more) as an electrode substance. Specific examples of such an electrode substance are constituted by a metal, an alloy, and an organic or inorganic conductive compound, or a mixture thereof. Specifically, a metal such as silver (Ag) or gold (Au), an oxide semiconductor such as copper iodide (CuI), ITO, ZnO, TiO₂, or SnO₂ are exemplified.

In addition, a material that is amorphous and able to be fabricated into a transparent conductive film such as IDIXO (In₂O₃—ZnO) may be used.

As the positive electrode, these electrode substances are formed into a thin film by a method such as vapor deposition or sputtering, and a pattern having a desired shape may be formed by photolithography or a pattern may be formed via a mask having a desired shape at the time of vapor deposition or sputtering of the electrode substance in a case in which the accuracy of pattern is not much required (about 100 μm or more).

Alternatively, it is also possible to use a wet film forming method such as a printing method or a coating method in the case of using a coatable substance such as an organic conductive compound. It is desirable to set the transmittance to be greater than 10% in the case of extracting the luminescence through this positive electrode, and the sheet resistance as a positive electrode is preferably several hundreds Ω/□ or less.

The film thickness of the positive electrode also depends on the material, but it is usually selected in a range of from 10 nm to 1 μm and preferably from 10 to 200 nm.

<<Negative Electrode>>

As the negative electrode (hereinafter, also referred to as the cathode), those are used which contain a metal (also referred to as an electron injecting metal), an alloy, an electrically conductive compound, and a mixture thereof which have a small work function (4 eV or less) as an electrode substance. Specific examples of such an electrode substance may include sodium, a sodium-potassium alloy, magnesium, lithium, a magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, aluminum, and a rare earth metal. Among these, a mixture of an electron injecting metal and a second metal of a metal which has a greater work function value than the electron injecting metal and is stable, for example, a magnesium/silver mixture, a magnesium/aluminum mixture, a magnesium/indium mixture, an aluminum/aluminum oxide (Al₂O₃) mixture, a lithium/aluminum mixture, or aluminum is suitable from the viewpoint of electron injection properties and durability against oxidation or the like. In addition, it is also possible to allow ITO to function as a negative electrode.

The negative electrode can be fabricated by forming these electrode substances into a thin film by a method such as vapor deposition or sputtering. In addition, the sheet resistance as a negative electrode is preferably several hundreds Ω/□ or less, and the film thickness is usually selected in a range of from 10 nm to 5 μm and preferably from 50 to 200 nm.

Incidentally, it is favorable that either of the positive electrode or negative electrode of the organic EL element is transparent or translucent in order to transmit the emitted light since the brightness is improved.

In addition, it is possible to fabricate a transparent or translucent negative electrode by fabricating a film of the metal on the negative electrode in a thickness of from 1 to 20 nm and then fabricating a film of the conductive transparent material exemplified in the description of the positive electrode thereon, and it is possible to fabricate an element in which both the positive electrode and the negative electrode exhibit transparency by applying this.

<<Supporting Substrate>>

As the supporting substrate (hereinafter, referred to as the base body, the substrate, the base material, the support, or the like) which can be used in the organic EL element of the present invention, the kind of the glass, plastic, or the like is not particularly limited, and it may be transparent or opaque. It is preferable that the supporting substrate is transparent in the case of extracting the light from the supporting substrate side. Examples of the preferably used transparent support substrate may include glass, quartz, and a transparent resin film. A particularly preferred supporting substrate is a resin film capable of imparting flexibility to the organic EL element.

Examples of the resin film may include a polyester such as polyethylene terephthalate (PET) or polyethylene naphthalate (PEN), polyethylene, polypropylene, a cellulose ester such as cellophane, cellulose diacetate, cellulose triacetate (TAC), cellulose acetate butyrate, cellulose acetate propionate (CAP), cellulose acetate phthalate, or cellulose nitrate or a derivative thereof, polyvinylidene chloride, polyvinyl alcohol, polyethylene vinyl alcohol, syndiotactic polystyrene, a polycarbonate, a norbornene resin, polymethylpentene, a polyether ketone, a polyimide, a polyether sulfone (PES), a polyphenylene sulfide, a polysulfone, a polyether imide, a polyether ketone imide, a polyamide, a fluorine resin, nylon, polymethyl methacrylate, an acrylate or a polyarylate, and a cycloolefin-based resin such as ARTON (trade name, manufactured by JSR Corporation) or APEL (trade name, manufactured by Mitsui Chemicals, Inc.).

A coat of an inorganic substance or an organic substance or a hybrid coat of the two may be formed on the surface of the resin film, and the resin film is preferably a barrier film which has a moisture vapor transmission rate (25±0.5° C., relative humidity of (90±2)% RH) measured by a method conforming to JIS K 7129-1992 of 0.01 g/m²·24 h or less, and further, it is preferably a high barrier film which has an oxygen transmission rate measured by a method conforming to JIS K 7126-1987 of 1×10⁻³ ml/m²·24 h·atm or less and a moisture vapor transmission rate of 1×10⁻⁵ g/m²·24 h or less.

As the material for forming a barrier film, a material having a function of suppressing the penetration of those that cause the deterioration of the element such as water or oxygen is desired, and it is possible to use, for example, silicon oxide, silicon dioxide, or silicon nitride. Furthermore, it is more preferable to have a stacked structure of these inorganic layers and a layer composed of an organic material in order to improve the brittleness of the film. The stacking order of the inorganic layer and the organic layer is not particularly limited, but it is preferable to alternately stack both layers plural times.

The method for forming the barrier film is not particularly limited, and it is possible to use, for example, a vacuum deposition method, a sputtering method, a reactive sputtering method, a molecular beam epitaxy method, a cluster ion beam method, an ion plating method, a plasma polymerization method, an atmospheric pressure plasma polymerization method, a plasma CVD method, a laser CVD method, a thermal CVD method, and a coating method, and it is even more preferable to form the barrier film by an atmospheric pressure plasma polymerization method as disclosed in JP 2004-68143 A.

Examples of the opaque supporting substrate may include a metal plate such as aluminum or stainless steel, a film or opaque resin substrate, a substrate made of ceramics.

The external extraction quantum efficiency of light emitted from the organic EL element of the present invention at room temperature is preferably 1% or more and more preferably 5% or more.

Here, it is external extraction quantum efficiency (%)=number of photon emitted outside organic EL element/number of electron flowed into organic EL element×100

In addition, a hue improving filter such as a color filter may be concurrently used, or a color conversion filter to convert the color of light emitted from the organic EL element into multiple colors by using a phosphor may be concurrently used.

<<Sealing>>

Examples of the sealing means used for sealing the organic EL element of the present invention may include a method in which the sealing member, the electrode, and the supporting substrate are bonded with an adhesive. The sealing member may be disposed so as to cover the display region of the organic EL element, and it may have a recessed plate shape or a flat plate shape. In addition, the transparency and electrical insulating properties thereof are not particularly limited.

Specifically, examples thereof may include a glass plate, a polymer plate or film, a metal plate or film. Examples of the glass plate may include particularly soda-lime glass, barium-strontium-containing glass, lead glass, aluminosilicate glass, borosilicate glass, barium borosilicate glass, and quartz. In addition, examples of the polymer plate may include a polycarbonate, an acrylate, polyethylene terephthalate, a polyether sulfide, and a polysulfone. Examples of the metal plate may include those composed of one or more kinds of metals selected from the group consisting of stainless steel, iron, copper, aluminum, magnesium, nickel, zinc, chromium, titanium, molybdenum, silicon, germanium, and tantalum or alloys thereof.

In the present invention, it is possible to preferably use a polymer film and a metal film for the fact that the organic EL element can be thinned. Furthermore, the polymer film is preferably one that has an oxygen transmission rate measured by a method conforming to JIS K 7126-1987 of 1×10⁻³ ml/m²·24 h or less and a moisture vapor transmission rate (25±0.5° C., relative humidity of (90±2)% RH) measured by a method conforming to JIS K 7129-1992 of 1×10⁻³ g/m²·24 h or less.

A sand blasting process, a chemical etching process, or the like is used in order to process the sealing member into a recessed shape.

Specific examples of the adhesive may include a photocuring and thermal curing type adhesive having a reactive vinyl group of an acrylic acid-based oligomer and a methacrylic acid-based oligomer, a moisture curing type adhesive such as an ester of 2-cyanoacrylic acid.

In addition, examples thereof may include a thermal and chemical curing type (two-liquid mixing) adhesive such as an epoxy-based one. In addition, examples thereof may include a polyamide, a polyester, and polyolefin of a hot-melt type. In addition, examples thereof may include a cationic curing type ultraviolet curable epoxy resin adhesive.

Incidentally, those which can be bonded and cured at from room temperature to 80° C. are preferable since the organic EL element is deteriorated by heat treatment in some cases. In addition, a drying agent may be dispersed in the adhesive. A commercially available dispenser may be used for coating the adhesive on the sealing portion, or the adhesive may be printed by screen printing.

In addition, it can be also suitable to form a layer of an inorganic substance or an organic substance in the form of being in contact with the support substrate by covering the electrode and the organic layer on the outside of the electrode on the side to sandwich the organic layer and to face the support substrate and to adopt the layer as the sealing film. In this case, as the material for forming the film, a material having a function of suppressing the penetration of those that cause the deterioration of the element such as water or oxygen is desired, and it is possible to use, for example, silicon oxide, silicon dioxide, or silicon nitride.

Furthermore, it is preferable to have a stacked structure of these inorganic layers and a layer composed of an organic material in order to improve the brittleness of the film. The method for forming these films is not particularly limited, and it is possible to use, for example, a vacuum deposition method, a sputtering method, a reactive sputtering method, a molecular beam epitaxy method, a cluster ion beam method, an ion plating method, a plasma polymerization method, an atmospheric pressure plasma polymerization method, a plasma CVD method, a laser CVD method, a thermal CVD method, and a coating method.

It is preferable to inject an inert gas such as nitrogen or argon or an inert liquid such as a fluorinated hydrocarbon or silicone oil as a gas phase and a liquid phase into the gap between the sealing member and the display region of the organic EL element. In addition, it is also possible to vacuum the gap. In addition, it is also possible to encapsulate a hygroscopic compound in the inside.

Examples of the hygroscopic compound may include a metal oxide (for example, sodium oxide, potassium oxide, calcium oxide, barium oxide, magnesium oxide, or aluminum oxide), a sulfate (for example, sodium sulfate, calcium sulfate, magnesium sulfate, or cobalt sulfate), a metal halide (for example, calcium chloride, magnesium chloride, cesium fluoride, tantalum fluoride, cerium bromide, magnesium bromide, barium iodide, or magnesium iodide), and a perchlorate (for example, barium perchlorate or magnesium perchlorate), and an anhydrous salt is suitably used as the sulfate, the metal halide, and the perchlorate.

<<Protective Film and Protective Plate>>

The outside of the sealing film or the film for sealing on the side to sandwich the organic layer and to face the support substrate may be provided with a protective film or a protective plate in order to increase the mechanical strength of the element. The mechanical strength is not always high particularly in a case in which the sealing is conducted using the sealing film, and thus it is preferable to provide such a protective film or a protective plate.

As the material can be used for this, it is possible to use a glass plate, a polymer plate or film, and a metal plate or film which are the same as those used in the sealing, but it is preferable to use a polymer film from the viewpoint of light weight and thinning.

<<Light Extraction Improving Technique>>

An organic EL element is generally said that light is emitted in the inside of a layer which has a higher refractive index (refractive index in a range of about from 1.6 to 2.1) than the air and only light to be about from 15 to 20% of the light generated in the luminous layer is extracted.

This is because the light incident on the interface (interface between the transparent substrate and the air) at an angle θ to be equal to or higher than the critical angle cannot be extracted to the outside of the element due to the total reflection or the total reflection of light is caused between the transparent electrode or the luminous layer and the transparent substrate so that the light is guided through the transparent electrode or the luminous layer, and as a result, the light escapes in the side direction of the element.

Examples of the method for improving the this light extraction efficiency may include a method in which a concave and a convex are formed on the transparent substrate surface to prevent the total reflection at the interface between the transparent substrate and the air (for example, U.S. Pat. No. 4,774,435), a method in which light collecting properties are imparted to the substrate to improve the efficiency (for example, JP 63-314795 A), a method in which a reflective surface is formed on the side surface or the like of the element (for example, JP 1-220394 A), a method in which a flat layer having an intermediate refractive index is introduced between the substrate and the luminous body to form an antireflective film (for example, JP 62-172691 A), a method in which a flat layer having a refractive index lower than that of the substrate is introduced between the substrate and the luminous body (for example, JP 2001-202827 A), and a method in which a diffraction grating is formed between the substrate and any layer of the transparent electrode layer or the luminous layer (including, between the substrate and the outside) (JP 11-283751 A).

In the present invention, it is possible to use these methods in combination with the organic electroluminescent element of the present invention, but it is possible to suitably use a method in which a flat layer having a refractive index lower than that of the substrate is introduced between the substrate and the luminous body and a method in which a diffraction grating is formed between the substrate and either of the transparent electrode layer or the luminous layer (including, between the substrate and the outside).

In the present invention, it is possible to obtain an element which has a higher brightness or exhibits excellent durability by combining these means.

The extraction efficiency of the light come out of the transparent electrode to the outside is higher as the refractive index of the medium is lower when a medium having a low refractive index is formed between the transparent electrode and the transparent substrate in a thickness longer than the wavelength of light.

Examples of the low refractive index layer may include aerogel, porous silica, magnesium fluoride, and a fluorine-based polymer. The refractive index of the transparent substrate is generally in a range of about from 1.5 to 1.7, and thus it is preferable that the low refractive index layer has a refractive index of about 1.5 or less. In addition, it is even more preferably 1.35 or less.

In addition, it is desirable that the thickness of the low refractive index medium is two or more times the wavelength in the medium. This is because the effect of the low refractive index layer is decreased when the thickness of the low refractive index medium is about the wavelength of light to be a layer thickness in which the electromagnetic waves oozed via evanescence enter into the substrate.

The method to introduce a diffraction grating into the interface to cause total reflection or into any medium has a feature that an effect of improving the light extraction efficiency is high. This method is intended to diffract the light that cannot be extracted to the outside due to the total reflection between the layers or the like and to extract the light to the outside by introducing a diffraction grating into between any layers or into a medium (inside the transparent substrate or inside the transparent electrode) so as to utilize the properties of the diffraction grating that can change the direction of light into a specific direction that is different from the refraction by the first order diffraction or the so-called Bragg diffraction of the second-order diffraction.

It is desirable that the diffraction grating to be introduced has a two-dimensional periodic refractive index. This is because the light emitted from the luminous layer is randomly generated in all directions, and thus only the light proceeding in a specific direction is diffracted and the extraction efficiency of light is not sufficiently increased when a general one-dimensional diffraction grating having a periodic refractive index distribution only in a certain direction is used.

However, the light proceeding in all directions is diffracted and the extraction efficiency of light is increased by changing the refractive index distribution to a two-dimensional distribution.

As the position into which the diffraction grating is introduced, the vicinity of the luminous layer where light is generated is desirable although the position may be between any layers or in a medium (inside the transparent substrate or inside the transparent electrode). At this time, the period of the diffraction grating is preferably in a range of about from ½ to 3 times the wavelength of light in the medium. As the arrangement of the diffraction grating, it is preferable that a two-dimensionally arrangement such as a square lattice shape, a triangular lattice shape, or a honeycomb lattice shape is repeated.

<<Light Collecting Sheet>>

It is possible to enhance the brightness in a specific direction as light is collected in a specific direction, for example, the front direction with respect to the luminous surface of the element by processing the organic EL element of the present invention so as to provide, for example, a microlens-arrayed structure on the light extracting side of the supporting substrate (substrate).

As an example of the microlens array, a quadrangular pyramid of which one side is 30 μm and the apex angle is 90 degrees is two-dimensionally arranged on the light extracting side of the substrate. One side is preferably in a range of 10 to 100 μm. It is preferable to set one side to be in this range since the effect of diffraction is generated so as to suppress coloring and the thickness does not also increase.

As the light collecting sheet, for example, it is possible to use those which have been put to practical use in an LED backlight of a liquid crystal display device. As such a sheet, for example, it is possible to use a brightness enhancing film (BEF) manufactured by 3M Japan Limited. The shape of the prism sheet may be, for example, those obtained by forming a Δ-shaped stripe having an apex angle of 90 degrees and a pitch 50 μm on the substrate, or it may be a shape in which the apex angle is rounded, a shape in which the pitch is randomly changed, or another shape.

In addition, the light diffusing plate or film may be concurrently used with the light collecting sheet in order to control the angle of light radiated from the organic EL element. For example, it is possible to use a diffusion film (light-up) manufactured by KIMOTO CO., LTD.

<Sequentially Layered Bottom Emission Type Organic EL Element 100>

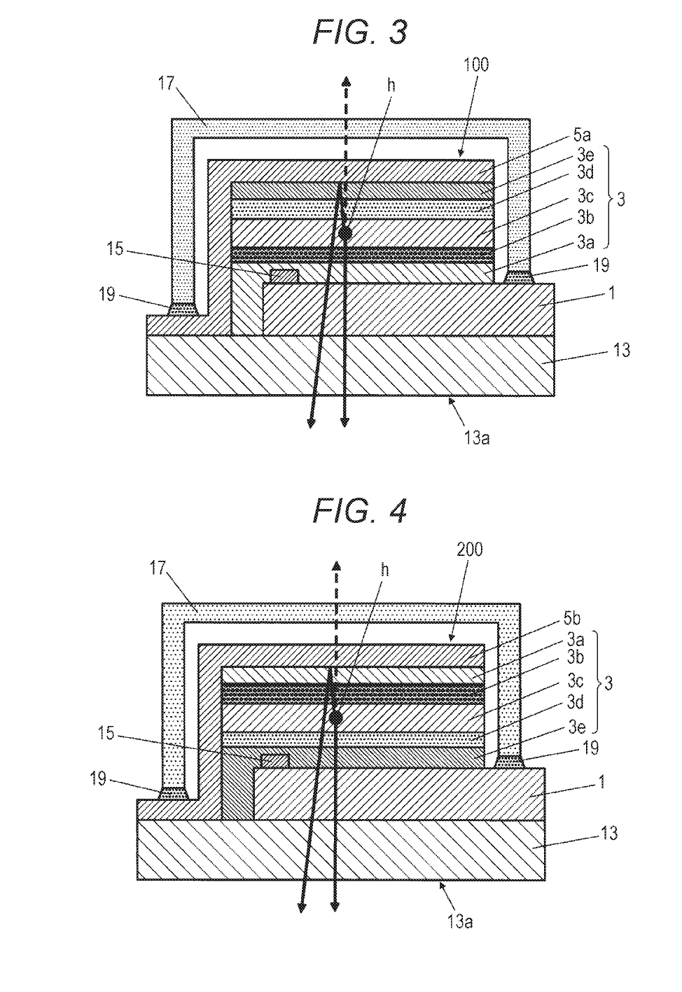

Here, as an example, a method for manufacturing a sequentially layered bottom emission type organic EL element 100 illustrated in FIG. 3 will be described.

First, a transparent electrode 1 composed of ITO as an anode (positive electrode) is fabricated on a transparent substrate 13.

Next, the films of a hole injection layer 3a, a hole transport layer 3b, a luminous layer 3c, an electron transport layer 3d, and an electron injection layer 3e are formed thereon in this order to form an organic layer 3. For the film formation of these layers, there are a spin coating method, a casting method, an ink-jet method, a vapor deposition method, and a printing method, but a vacuum deposition method or a spin coating method is even more preferable from the viewpoint of being likely to obtain a uniform film and of hardly generating pinholes. Furthermore, different film forming methods may be applied for each layer.

In the case of employing the vapor deposition method for the film formation of these layers, the vapor deposition conditions vary depending on the kinds of the compounds to be used, but in general, it is desirable to appropriately select in a range in which the boat heating temperature is from 50 to 450° C., the vacuum degree is from 1×10⁻⁶ to 1×10⁻² Pa, the deposition rate is from 0.01 to 50 nm/sec, the substrate temperature is from −50 to 300° C., the layer thickness is from 0.1 to 5 m.

After the organic layer 3 is formed as described above, a counter electrode 5a to be a cathode (negative electrode) is formed on the upper part thereof by a suitable film forming method such as a vapor deposition method or a sputtering method. In this case, the counter electrode 5a is patterned into a shape in which the terminal portion is pulled out from above the organic layer 3 to the periphery of the transparent substrate 13 while maintaining an insulating state with respect to the transparent electrode 1 by the organic layer 3. By this, the organic EL element 100 is obtained. Thereafter, the organic EL element 100 is provided with a sealing member 17 to cover at least the organic layer 3 in a state of exposing the transparent electrode 1 and the terminal portion of the counter electrode 5a.

Hence, a desired organic EL element is obtained on the transparent substrate 13. In the fabrication of such an organic EL element 100, it is preferable to consistently fabricate from the organic layer 3 to the counter electrode 5a by vacuum drawing of one time, but the transparent substrate 13 may be taken out from the vacuum atmosphere in the middle of the fabrication so as to be subjected to a different film forming method. In that case, it is required to conduct the operation in a dry inert gas atmosphere.

In a case in which a DC voltage is applied to the organic EL element 100 thus obtained, it is possible to observe luminescence when the polarity of the transparent electrode 1 that is the anode is set to + and the polarity of the counter electrode 5a that is the cathode is set to − and a voltage of about from 2 to 40 V is applied. In addition, an AC voltage may be applied. Incidentally, the waveform of the alternating current to be applied may be arbitrary.

Here, the transparent electrode 1 is composed of a metal, an alloy, an organic or inorganic conductive compound, a mixture thereof, or the like which is used as the anode (positive electrode) described above. Specifically, a metal thin film (thickness of from 1 to 50 nm) of silver (Ag) or gold (Au), and an oxide semiconductor such as ITO, ZnO, TiO₂, or SnO₂ are exemplified.

Here, the counter electrode 5a can be composed of a metal, an alloy, an organic or inorganic conductive compound, and a mixture thereof which are used as the cathode (negative electrode). Specifically, it is possible to use aluminum, silver, magnesium, lithium, a magnesium/copper mixture, a magnesium/silver mixture, a magnesium/aluminum mixture, a magnesium/indium mixture, indium, a lithium/aluminum mixture, a rare earth metal, and an oxide semiconductor such as indium-doped tin oxide, ZnO, TiO₂, or SnO₂.

<Effect of Organic EL Element>

The organic EL element 100 described above has a constitution in which the transparent electrode 1 equipped with both optical transparency and conductivity is used as the anode and the organic layer 3 and the counter electrode 5a to be the cathode are provided on the upper part thereof. Hence, it is possible to achieve a higher brightness due to the improvement in extraction efficiency of emitted light h from the transparent electrode 1 side while realizing highly bright luminescence of the organic EL element 100 by applying a sufficient voltage to between the transparent electrode 1 and the counter electrode 5a. Furthermore, it is also possible to achieve the improvement in luminous lifespan due to a decrease in driving voltage for obtaining a predetermined brightness.

<Reversely Layered Bottom Emission Type Organic EL Element 200>

FIG. 4 is a schematic cross-sectional diagram illustrating an example of a reversely layered bottom emission type organic EL element. An organic EL element 200 illustrated in FIG. 4 differs from the organic EL element 100 of a sequentially layered constitution illustrated in FIG. 3 in that the transparent electrode 1 is used as the cathode (negative electrode).

Hereinafter, the characteristic constitution of the reversely layered type organic EL element 200 will be described, and the overlapping detailed description on the same constituents as those in the sequentially layered constitution will be omitted.

As illustrated in FIG. 4, the organic EL element 200 is provided on the transparent substrate 13, and the transparent electrode 1 described previously is used as the transparent electrode 1 on the transparent substrate 13 in the same manner as in the organic EL element 100. Hence, the organic EL element 200 is constituted so as to extract the emitted light h at least from the transparent substrate 13 side. However, this transparent electrode 1 is used as the cathode (negative electrode). Hence, a counter electrode 5b is used as the anode.

It is the same as in the organic EL element 100 that the layer structure of the organic EL element 200 thus constituted is not limited to the example to be described below and it may have a general layer structure.

As an example in the case of the organic EL element 200, a constitution in which an electron injection layer 3e/an electron transport layer 3d/a luminous layer 3c/a hole transport layer 3b/a hole injection layer 3a are stacked on the upper part of the transparent electrode 1 which functions as the cathode in this order is exemplified. Provided that, it is essential to have at least the luminous layer 3c composed of an organic material among these.

Incidentally, various constitutions are employed in the organic layer 3 if necessary in addition to these layers in the same manner as that described in the organic EL element 100. It is also the same as in the organic EL element 100 that only the portion where the organic layer 3 is sandwiched between the transparent electrode 1 and the counter electrode 5b is the luminous region in the organic EL element 200 in such a constitution.

In addition, it is also the same as in the organic EL element 100 that an auxiliary electrode 15 may be provided so as to be in contact with the transparent electrode 1 for the purpose of achieving a low resistance of the transparent electrode 1 in the layer constitution as described above.

Here, it is possible to appropriately use the material that is used as the anode and described above in the counter electrode 5b. In addition, it is possible to appropriately use the material that is used as the cathode and described above in the transparent electrode 1 as well.

The counter electrode 5b that is constituted as described above can be fabricated by forming these conductive materials into a thin film by a method such as vapor deposition or sputtering.

In addition, the sheet resistance as the counter electrode 5b is preferably several hundreds Ω/□ or less, and the film thickness is usually from 1 nm to 5 μm and preferably from 5 to 200 nm.

Incidentally, in a case in which the organic EL element 200 is constituted so as to extract the emitted light h from the counter electrode 5b side, a conductive material exhibiting favorable optical transparency is selected among the conductive materials described above and used as the material constituting the counter electrode 5b.

The organic EL element 200 having a constitution as described above is sealed with a sealing member 17 in the same manner as in the organic EL element 100 for the purpose of preventing the organic layer 3 from deteriorating.

Among the major layers constituting the organic EL element 200 described above, the detailed constitution of the constituents other than the counter electrode 5b used as the anode and the method for manufacturing the organic EL element 200 are the same as those in the organic EL element 100. Hence, the detailed description thereon is omitted.

<Effect of Organic EL Element>

The organic EL element 200 described above has a constitution in which the transparent electrode 1 equipped with both optical transparency and conductivity is used as the cathode and the organic layer 3 and the counter electrode 5b to be the anode are provided on the upper part thereof. Hence, it is possible to achieve a higher brightness due to the improvement in extraction efficiency of emitted light h from the transparent electrode 1 side while realizing highly bright luminescence of the organic EL element 200 by applying a sufficient voltage to between the transparent electrode 1 and the counter electrode 5b in the same manner as in the organic EL element 100. Furthermore, it is also possible to achieve the improvement in luminous lifespan due to a decrease in driving voltage for obtaining a predetermined brightness.

<Sequentially Layered Top Emission Type Organic EL Element 300>

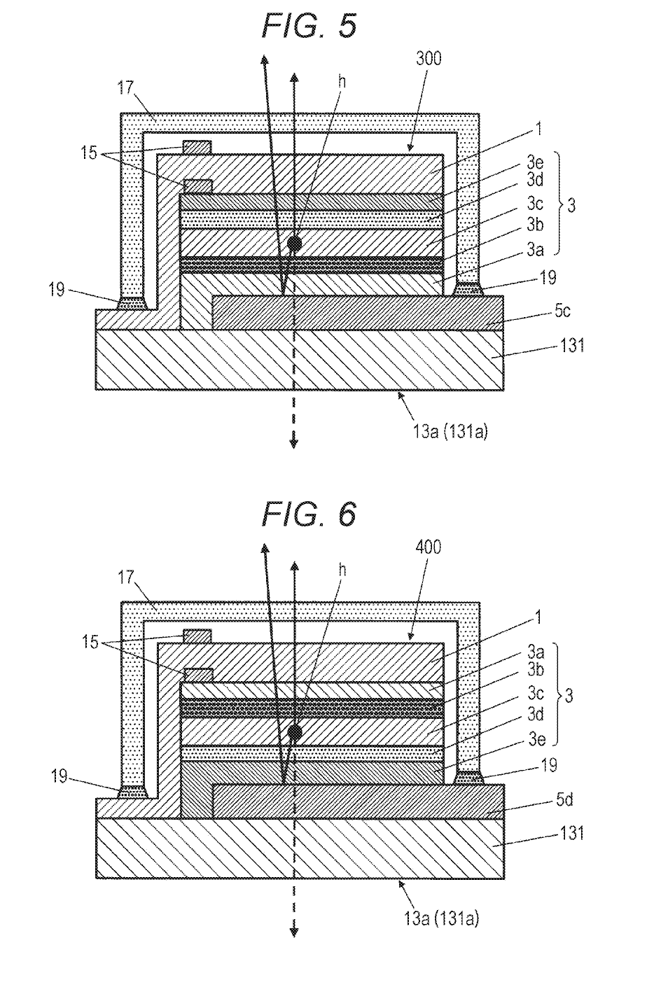

FIG. 5 is a schematic cross-sectional diagram illustrating a sequentially layered top emission type organic EL element 300 as an example of the organic EL element of the present invention. The organic EL element 300 illustrated in FIG. 5 differs from the sequentially layered bottom emission type organic EL element 100 illustrated in FIG. 3 in that a counter electrode 5c is provided on the substrate 131 side and the organic layer 3 and the transparent electrode 1 are stacked on the upper part thereof in this order.

Hereinafter, the characteristic constitution of the sequentially layered top emission type organic EL element 300 will be described, and the overlapping detailed description on the same constituents as those in the organic EL element 100 will be omitted.

The organic EL element 300 illustrated in FIG. 5 is provided on a substrate 131, and the counter electrode 5c to be the anode, the organic layer 3, and the transparent electrode 1 to be the cathode are stacked in this order from the substrate 131 side. Among them, the transparent electrode 1 described previously is used as the transparent electrode 1. Hence, the organic EL element 300 is constituted so as to extract the emitted light h from the transparent electrode 1 side opposite to at least the substrate 131.

It is the same as in the organic EL element 100 that the layer structure of the organic EL element 300 thus constituted is not limited to the example to be described below and it may have a general layer structure.

As an example in the case of the organic EL element 300, a constitution in which a hole injection layer 3a/a hole transport layer 3b/a luminous layer 3c/an electron transport layer 3d/an electron injection layer 3e are stacked on the upper part of the counter electrode 5c which functions as the anode in this order is exemplified.

Incidentally, various constitutions are employed in the organic layer 3 if necessary in addition to these layers in the same manner as that described in the organic EL element 100. It is the same as in the organic EL element 100 that only the portion where the organic layer 3 is sandwiched between the transparent electrode 1 and the counter electrode 5c is to be the luminous region in the organic EL element 300 in such a constitution.

In addition, it is also the same as in the organic EL element 100 that an auxiliary electrode 15 may be provided so as to be in contact with the transparent electrode 1 for the purpose of achieving a low resistance of the transparent electrode 1 in the layer constitution as described above.

Here, it is possible to appropriately use the material that is used as the anode and described above in the counter electrode 5c. In addition, it is possible to appropriately use the material that is used as the cathode and described above in the transparent electrode 1 as well.

The counter electrode 5c that is constituted as described above can be fabricated by forming these conductive materials into a thin film by a method such as vapor deposition or sputtering.

In addition, the sheet resistance as the counter electrode 5c is preferably several hundreds Ω/□ or less, and the film thickness is usually from 1 nm to 5 μm and preferably from 5 to 200 nm.

Incidentally, in a case in which the organic EL element 300 is constituted so as to extract the emitted light h from the counter electrode 5c side as well, a conductive material exhibiting favorable optical transparency is selected among the conductive materials described above and used as the material constituting the counter electrode 5c. In addition, in this case, one that is the same as the transparent substrate 13 described in the organic EL element 100 is used as the substrate 131, and the surface facing the outside of the substrate 131 is to be a light extracting surface 131a.

<Effect of Organic EL Element>

The organic EL element 300 described above has a constitution in which the transparent electrode 1 is provided on the upper part of the electron injection layer 3e constituting the uppermost part of the organic layer 3 as the cathode (negative electrode). Hence, it is possible to achieve a higher brightness due to the improvement in extraction efficiency of emitted light h from the transparent electrode 1 side while realizing highly bright luminescence of the organic EL element 300 by applying a sufficient voltage to between the transparent electrode 1 and the counter electrode 5c in the same manner as in the organic EL element 100 and the organic EL element 200. Furthermore, it is also possible to achieve the improvement in luminous lifespan due to a decrease in driving voltage for obtaining a predetermined brightness. In addition, it is possible to extract the emitted light h from the counter electrode 5c as well in a case in which the counter electrode 5c exhibits optical transparency. Incidentally, it is also possible to extract the luminescence with improved color purity by the microcavity effect in a case in which the counter electrode 5c is translucent.

<Reversely Layered Top Emission Type Organic EL Element 400>

FIG. 6 is a schematic cross-sectional diagram illustrating a reversely layered top emission type organic EL element 400 as an example of the organic EL element of the present invention. The organic EL element 400 illustrated in FIG. 6 differs from the reversely layered bottom emission type organic EL element 100 illustrated in FIG. 4 in that a counter electrode 5d is provided on the substrate 131 side and the organic layer 3 and the transparent electrode 1 are stacked on the upper part thereof in this order.

Hereinafter, the characteristic constitution of the reversely layered top emission type organic EL element 400 will be described, and the overlapping detailed description on the same constituents as those in the organic EL element 100 will be omitted.

The organic EL element 400 illustrated in FIG. 5 is provided on a substrate 131, and the counter electrode 5d to be the cathode, the organic layer 3, and the transparent electrode 1 to be the anode are stacked in this order from the substrate 131 side. Among these, the transparent electrode 1 described previously is used as the transparent electrode 1. Hence, the organic EL element 400 is constituted so as to extract the emitted light h from the transparent electrode 1 side opposite to at least the substrate 131.

It is the same as in the organic EL element 100 that the layer structure of the organic EL element 400 thus constituted is not limited to the example to be described below and it may have a general layer structure.

As an example in the case of the organic EL element 400, a constitution in which an electron injection layer 3e/an electron transport layer 3d/a luminous layer 3c/a hole transport layer 3b/a hole injection layer 3a are stacked on the upper part of the counter electrode 5d which functions as the cathode in this order is exemplified.

Incidentally, various constitutions are employed in the organic layer 3 if necessary in addition to these layers in the same manner as that described in the organic EL element 100. It is the same as in the organic EL element 100 that only the portion where the organic layer 3 is sandwiched between the transparent electrode 1 and the counter electrode 5d is to be the luminous region in the organic EL element 400 in such a constitution.

In addition, it is also the same as in the organic EL element 100 that an auxiliary electrode 15 may be provided so as to be in contact with the transparent electrode 1 for the purpose of achieving a low resistance of the transparent electrode 1 in the layer constitution as described above.

Furthermore, it is possible to appropriately use the material that is used as the cathode in the counter electrode 5d. In addition, it is possible to appropriately use the material that is used as the anode and described above in the transparent electrode 1 as well.

The counter electrode 5d that is constituted as described above can be fabricated by forming these conductive materials into a thin film by a method such as vapor deposition or sputtering.

In addition, the sheet resistance as the counter electrode 5d is preferably several hundreds Ω/□ or less, and the film thickness is usually from 5 nm to 5 μm and preferably from 5 to 200 nm.

Incidentally, in a case in which the organic EL element 400 is constituted so as to extract the emitted light h from the counter electrode 5d side as well, a conductive material exhibiting favorable optical transparency is selected among the conductive materials described above and used as the material constituting the counter electrode 5d. In addition, in this case, one that is the same as the transparent substrate 13 described in the organic EL element 100 is used as the substrate 131, and the surface facing the outside of the substrate 131 is to be a light extracting surface 131a.

<Effect of Organic EL Element>

The organic EL element 400 described above has a constitution in which the transparent electrode 1 is provided on the upper part of the hole injection layer 3a constituting the uppermost part of the organic layer 3 as the anode. Hence, it is possible to achieve a higher brightness due to the improvement in extraction efficiency of emitted light h from the transparent electrode 1 side while realizing highly bright luminescence of the organic EL element 400 by applying a sufficient voltage to between the transparent electrode 1 and the counter electrode 5d in the same manner as in the organic EL elements 100 to 300. Furthermore, it is also possible to achieve the improvement in luminous lifespan due to a decrease in driving voltage for obtaining a predetermined brightness. In addition, it is possible to extract the emitted light h from the counter electrode 5d as well in a case in which the counter electrode 5d exhibits optical transparency. Incidentally, it is also possible to extract the luminescence with improved color purity by the microcavity effect in a case in which the counter electrode 5d is translucent.

<<Applications>>

The organic EL element of the present invention is preferably equipped in a display device. In addition, it is possible to use the organic EL element as a display and a light source for various kinds of luminescence as well.

Examples of the light source for luminescence may include a lighting device (home lighting, vehicle interior lighting), a watch, or a backlight for liquid crystal, a billboard, a traffic light, a light source for an optical storage medium, a light source for an electrophotographic copying machine, a light source for an optical communication processing machine, and a light source for an optical sensor. Although it is not limited to these, the organic EL element of the present invention can be effectively used particularly in the applications as a backlight for a liquid crystal display device and a light source for lighting.

In the organic EL element of the present invention, the patterning may be conducted using a metal mask or an ink-jet printing method at the time of film formation if necessary. In the case of conducting the patterning, only the electrode may be patterned, the electrode and the luminous layer may be patterned, or all the layers of the element may be patterned, and it is possible to use a method known in the prior art in the fabrication of the element.

<<Display Device>>

The display device of the present invention will be described. The display device of the present invention is equipped with the organic EL element of the present invention. The display device of the present invention may be in a single color or multiple colors, but a multicolor display device will be described herein.

In the case of a multicolor display device, it is possible to form a film on the entire surface by a vapor deposition method, a casting method, a spin coating method, an ink-jet method, a printing method, or the like by providing the shadow mask only at the time of forming the luminous layer.

In the case of patterning only the luminous layer, although the method therefor is not limited, but a vapor deposition method, an ink-jet method, a spin coating method, and a printing method are preferable.

The constitution of the organic EL element to be provided in a display device is selected from the constitutional examples of the organic EL element described above if necessary.

In addition, the manufacturing method of the organic EL element is as presented in an aspect of the manufacture of the organic EL element of the present invention described above.

In the case of applying a DC voltage to the multicolor display device thus obtained, it is possible to observe luminescence as the polarity of the anode and the cathode is set to + and −, respectively, and a voltage of about from 2 to 40 V is applied. In addition, the current does not flow and luminescence does not occur at all even when a voltage is applied if the polarity is reversed. Furthermore, luminescence is observed only in a state in which the anode is + and the cathode is − in the case of applying an AC voltage. Incidentally, the waveform of the alternating current to be applied may be arbitrary.

The multicolor display device can be used as a display device, a display, and a light source for various kinds of luminescence. In the display device and the display, it is possible to display full color by using three kinds of organic EL elements that emit blue light, red light, and green light, respectively.

Examples of the display device and the display may include a television, a personal computer, mobile equipment, AV equipment, a display for text broadcasting, and an information display used in a vehicle. It may be used as a display device particularly for reproducing a still image or a moving image, and the driving system in the case of being used as a display device for reproducing a moving image may be either of a simple matrix (passive matrix) system or an active matrix system.

Examples of the light source for luminescence may include home lighting, vehicle interior lighting, a watch, or a backlight for liquid crystal, a billboard, a traffic light, a light source for an optical storage medium, a light source for an electrophotographic copying machine, a light source for an optical communication processing machine, and a light source for an optical sensor, but the present invention is not limited thereto.

<<Lighting Device>>

The lighting device of the present invention will be described. The lighting device of the present invention has the organic EL element described above.

It may be used as an organic EL element obtained by equipping a resonator structure to the organic EL element of the present invention, and examples of the intended use of such an organic EL element having a resonator structure may include a light source for an optical storage medium, a light source for an electrophotographic copying machine, a light source for an optical communication processing machine, and a light source for an optical sensor, but it is not limited thereto. The organic EL element of the present invention may also be used in the above applications after being subjected to the laser oscillation.

In addition, the organic EL element of the present invention may be used as a kind of lamp such as a light source for lighting or exposure, or it may be used as a projection device of a type to project an image or a display device (display) of a type to directly view a still image or a moving image.

The driving system in the case of being used as a display device for reproducing a moving image may be either of a simple matrix (passive matrix) system or an active matrix system. Alternatively, it is possible to fabricate a full-color display device by using two or more kinds of the organic EL elements of the present invention having different luminous colors.

In addition, an iridium complex which can be used as the phosphorescence emitting compound in the present invention can be applied to an organic EL element which emits substantially white light as a lighting device. Light of a plurality of luminous colors is emitted at the same time by a plurality of luminescent materials so as to obtain white luminescence by color mixing. The combination of a plurality of luminous colors may be one that contains three maximum luminescent wavelengths of the three primary colors of red, green, and blue or one that contains two maximum luminescent wavelengths utilizing the complementary color relation between blue and yellow, blue-green and orange, or the like.

In addition, the combination of the luminescent materials for obtaining a plurality of luminous colors may be either of the combination of a plurality of materials which emit light in a plurality of phosphorescence or fluorescence or the combination of a luminescent material which emits light in fluorescence or phosphorescence with a coloring material which emits the light from the luminescent material as excitation light.

The mask is provided only at the time of forming the luminous layer, the hole transport layer, the electron transport layer, or the like, it may be simply disposed so as to coat and separate using the mask, the other layers are common so that the patterning of the mask or the like is not required, for example an electrode film can be formed on the entire surface by a vapor deposition method, a casting method, a spin coating method, an ink-jet method, a printing method, and the like, and the productivity is also improved.

According to this method, the element itself emits white light unlike a white organic EL device having luminescent elements of a plurality of colors disposed parallel in an array shape.

The luminescent material used for the luminous layer is not particularly limited, and for example, in the case of the backlight for a liquid crystal display element, a white color may be obtained by combining the metal complex used in the present invention with an arbitrary one selected among known luminescent materials so as to fall in the wavelength range corresponding to the CF (color filter) characteristics.

<<One Aspect of Lighting Device of Present Invention>>

An aspect of the lighting device of the present invention that is equipped with the organic EL element of the present invention will be described.

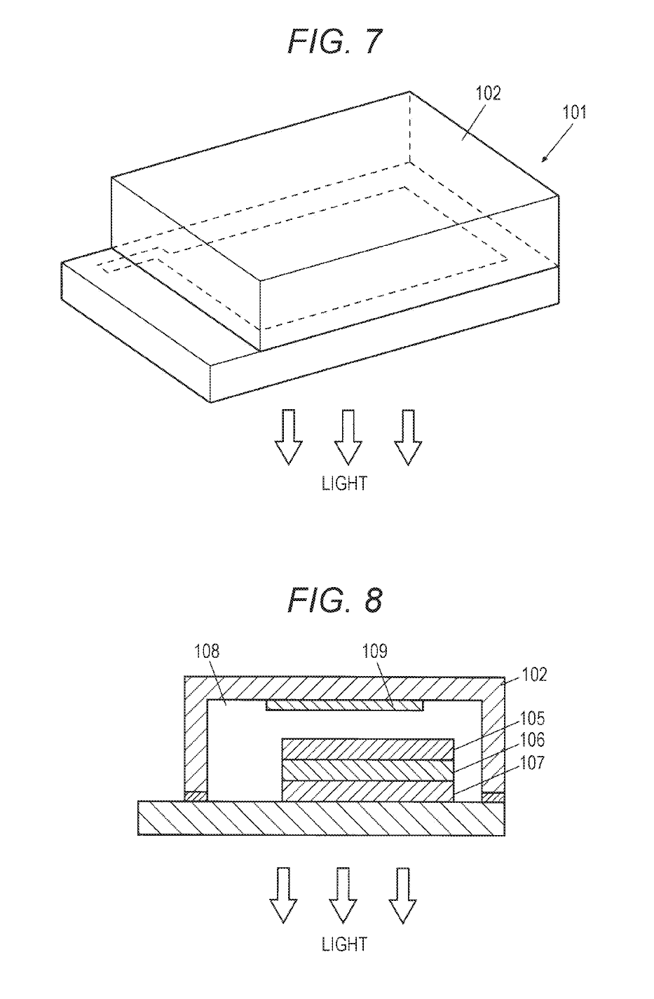

The nonluminescent surface of the organic EL element of the present invention is covered with a glass case, an epoxy-based photocurable adhesive (LUXTRACK LC0629B manufactured by TOAGOSEI CO., LTD.) as a sealing material is applied on the circumference of a glass substrate having a thickness of 300 μm used as a substrate for sealing, this is superimposed on the negative electrode and brought into close contact with the transparent support substrate, the epoxy-based photocurable adhesive is cured by being irradiated with UV light from the glass substrate side to seal the organic EL element, whereby it is possible to form the lighting device as illustrated in FIG. 7 and FIG. 8.

FIG. 7 illustrates an outline diagram of a lighting device, and the organic EL element of the present invention (an organic EL element 101 in the lighting device) is covered with a glass cover 102 (incidentally, the sealing operation using the glass cover was conducted in a glove box in a nitrogen atmosphere (in an atmosphere of highly pure nitrogen gas having a purity of 99.999% or higher) without allowing the organic EL element 101 in the lighting device to contact with the air).

FIG. 8 illustrates a cross-sectional diagram of a lighting device, and in FIG. 8, 105 denotes a counter electrode, 106 denotes an organic layer, 107 denotes a glass substrate with transparent electrode. As described above, it is determined by the stacking order of the organic which of the transparent electrode 107 and the counter electrode 105 serves as the negative electrode and the positive electrode, respectively. Incidentally, in a glass cover 102, a nitrogen gas 108 is filled and a water trapping agent 109 is provided.

Hence, the organic EL element of the present invention is suitably equipped in a lighting device.

### EXAMPLES

Hereinafter, the present invention will be specifically described with reference to Examples, but the present invention is not limited thereto. Incidentally, the term “parts” or “%” used in Examples indicates the term “parts by mass” or “% by mass” unless otherwise stated.

### Example 1

### Fabrication of Reversely Layered Type Blue Phosphorescent Organic EL Element

(1) Fabrication of Organic EL Element 1-1

A substrate (NA45 manufactured by NH Techno Glass Corp.) obtained by forming a film of ITO as the negative electrode on a glass substrate of 100 mm×100 mm×1.1 mm in 100 nm was patterned. Thereafter, this transparent substrate provided with an ITO transparent electrode was subjected to the ultrasonic cleaning with isopropyl alcohol, dried with a dry nitrogen gas, and subjected to the UV ozone cleaning for 5 minutes.

This transparent substrate was fixed to the substrate holder of a commercially available RF sputtering apparatus. An n-type amorphous oxide semiconductor obtained by molding C12A7 into a flat plate with reference to the method described in JP 2013-40088 A was used as the sputtering target.

For the sputtering, a film was formed in an atmosphere of argon gas at 500 mPa at a substrate temperature of room temperature (25° C.) and an input power of 100 W, thereby obtaining an electron injection layer (EIL layer) composed of a C12A7 thin film having a film thickness of 10 nm.

Incidentally, it has been confirmed that the C12A7 thin film is a C12A7 film which is in an amorphous state and has a broad spectrum by the XRD measurement of the C12A7 thin film for analysis formed at the same time.

The concentration of electrons of this C12A7 thin film measured by the method described in Patent Literature 1 was 1.0×10²¹/cm³. In addition, the work function measured by UPS was 3.0 eV.

Subsequently, the electron injection layer was transferred to a vacuum deposition apparatus without being exposed to the air and fixed to the substrate holder of the vacuum deposition apparatus.

The materials constituting the respective layers were filled in the respective crucibles for vapor deposition in the vacuum deposition apparatus in the respective optimum amounts for the fabrication of the element. One that was fabricated with a material for resistance heating made of molybdenum or tungsten was used as the crucible for vapor deposition.

After the pressure of the vacuum deposition apparatus was decreased to a vacuum degree of 1×10⁻⁴ Pa, and Alq₃ was heated by energizing the crucible for vapor deposition containing Alq₃ and deposited on the electron injection layer composed of C12A7 at a deposition rate of 0.1 nm/sec, thereby forming an electron transport layer having a layer thickness of 20 nm.

Subsequently, Compound H-1 and Compound BD-1 were co-deposited on the electron transport layer at a deposition rate of 0.1 nm/sec and 0.006 nm/sec, respectively, to provide a luminous layer having a layer thickness of 40 nm.

Subsequently, α-NPD was deposited thereon at a deposition rate of 0.1 nm/sec to form a hole transport layer having a layer thickness of 70 nm.

Thereafter, HAT was deposited thereon at a deposition rate of 0.1 nm/sec to form a hole injection layer having a layer thickness of 10 nm.

Furthermore, aluminum was deposited thereon in 100 nm to form a positive electrode.

The nonluminescent surface side of the element was covered with a can-shaped glass case in an atmosphere of highly pure nitrogen gas having a purity of 99.999% or higher, and the electrode lead-out wiring was installed thereto, thereby fabricating the organic EL element 1-1.

The compounds used in the present Example are those having the following Chemical Structural Formulas.

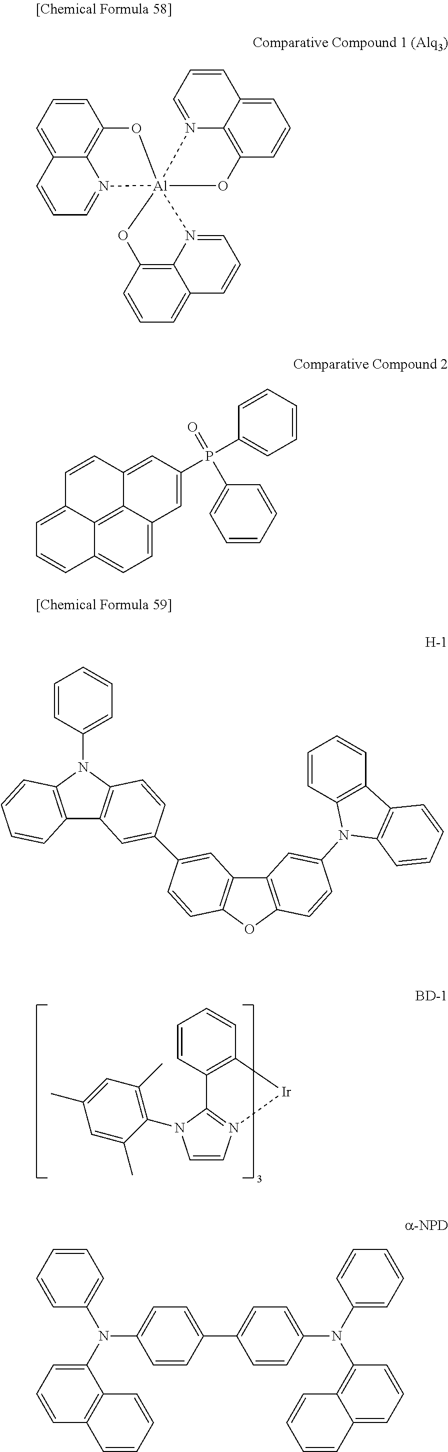

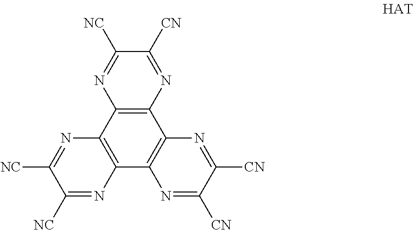 [SMILES: N#Cc1nc2c3nc(C#N)c(C#N)nc3c3nc(C#N)c(C#N)nc3c2nc1C#N]

(2) Fabrication of Organic EL Elements 1-2 to 1-26

The organic EL elements 1-2 to 1-24 were fabricated in the same manner except that the compounds presented in Table 1 were used instead of the compound Alq₃ of the electron transport layer in the fabrication of the organic EL element 1-1.

Incidentally, the electron transport layer used in the organic EL elements 1-25 and 1-26 was a polymer material, and thus it was formed by spin coating the polymer material on the electron injection layer composed of C12A7 under the following conditions in a glove box under the following conditions.

<Electron Transport Layer Coating Solution of EL Element 1-25>

ET-201: 15 mg

Dehydrated 1,1,1,3,3,3-hexafluoroisopropanol: 3 ml

The dissolved solution was formed into a film by a spin coating method under conditions of 1000 rpm and 30 seconds, and the film was dried by heating at 120° C. for 1 hour in a glove box, thereby providing an electron transport layer having a layer thickness of 20 nm.

The organic EL element 1-26 was fabricated in the same manner as in the organic EL element 1-25 except that ET-201 was changed to ET-216.

(3) Evaluation of Organic EL Elements 1-1 to 1-26

(3-1) Luminous Efficiency (Relative Value)

The organic EL element was lighted up at room temperature (25° C.) under a constant current condition of 2.5 mA/cm², and the luminescent brightness (L) [cd/m²] was measured immediately after the start of lighting up to calculate the external extraction quantum efficiency (i). Here, the measurement of the luminescent brightness was conducted using the CS-1000 (manufactured by Konica Minolta, Inc.), and the external extraction quantum efficiency was expressed as a relative value to 100 for the organic EL element 1-1. It indicates that the luminous efficiency is higher than Comparative Example and preferable as the relative value of the external extraction quantum efficiency is greater.

(3-2) Initial Driving Voltage

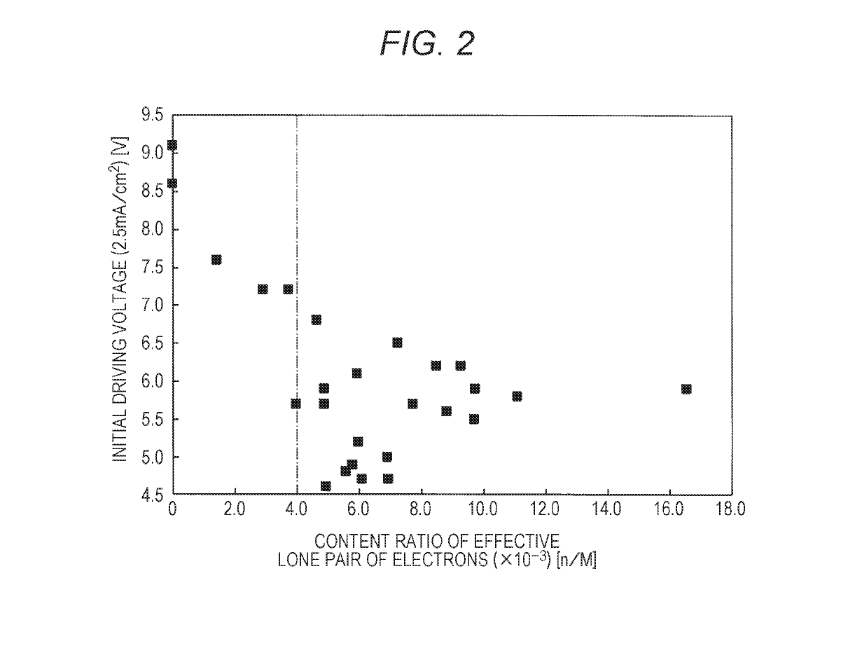

The initial voltage when the organic EL element was driven at room temperature (25° C.) under a constant current condition of 2.5 mA/cm² was measured and adopted as the initial driving voltage. In addition, the relation between the content ratio [n/M] of effective lone pair of electrons and the driving voltage is illustrated in FIG. 2.

(3-3) Half-Life (Relative Value)

The half-life was evaluated according to the following measurement method.

The respective organic EL elements were subjected to constant current driving at a current to provide an initial brightness of 1000 cd/m², the time until the brightness reached ½ (500 cd/m²) of the initial brightness was determined, and this was adopted as a measure of the half-life.

Incidentally, the half-life was expressed as a relative value to 100 for the organic EL element 1-1. It indicates that the durability is higher than Comparison and preferable as the relative value of the half-life is greater.

TABLE 1

| | | | | Content | | | | |
|---|---|---|---|---|---|---|---|---|
| | | | | ratio of | | | | |
| | | Number of | | effective | | | | |
| | | effective | | lone pair of | Luminous | Initial | | |
| Organic | Electron | lone pair of | Molecular | electrons | efficiency | driving | Half-life | |
| EL | transporting | electrons | weight | (×10⁻³) | (relative | voltage | (relative | |
| element | material | [n] | [M] | [n/M] | value) | (V) | value) | Remarks |
| 1-1 | Comparative | 0 | 459 | 0 | 100 | 9.1 | 100 | Comparative |
| | Compound 1 | | | | | | | Example |
| 1-2 | Comparative | 0 | 402 | 0 | 94 | 8.6 | 73 | Comparative |
| | Compound 2 | | | | | | | Example |
| 1-3 | ET-10 | 4 | 717 | 5.6 | 166 | 4.8 | 412 | Present invention |
| 1-4 | ET-22 | 4 | 807 | 5.0 | 167 | 4.6 | 366 | Present invention |
| 1-5 | ET-113 | 6 | 617 | 9.7 | 138 | 5.9 | 186 | Present invention |
| 1-6 | ET-124 | 2 | 410 | 4.9 | 144 | 5.9 | 229 | Present invention |
| 1-7 | ET-125 | 6 | 1036 | 5.8 | 161 | 4.9 | 394 | Present invention |
| 1-8 | ET-127 | 4 | 517 | 7.7 | 142 | 5.7 | 214 | Present invention |
| 1-9 | ET-129 | 6 | 647 | 9.3 | 137 | 6.2 | 197 | Present invention |
| 1-10 | ET-130 | 4 | 412 | 9.7 | 147 | 5.5 | 226 | Present invention |
| 1-11 | ET-132 | 6 | 541 | 11.0 | 133 | 5.8 | 192 | Present invention |
| 1-12 | ET-133 | 9 | 544 | 17.0 | 135 | 5.9 | 181 | Present invention |
| 1-13 | ET-144 | 5 | 718 | 7.0 | 162 | 4.7 | 353 | Present invention |
| 1-14 | ET-151 | 3 | 502 | 6.0 | 159 | 5.2 | 281 | Present invention |
| 1-15 | ET-158 | 4 | 578 | 6.9 | 154 | 5.0 | 273 | Present invention |
| 1-16 | ET-162 | 4 | 655 | 6.1 | 165 | 4.7 | 404 | Present invention |
| 1-17 | ET-167 | 2 | 535 | 3.7 | 123 | 7.2 | 166 | Present invention |
| 1-18 | ET-175 | 4 | 673 | 5.9 | 154 | 6.1 | 190 | Present invention |
| 1-19 | ET-184 | 4 | 552 | 7.2 | 129 | 6.5 | 174 | Present invention |
| 1-20 | ET-186 | 2 | 685 | 2.9 | 129 | 7.2 | 172 | Present invention |
| 1-21 | ET-193 | 5 | 566 | 8.8 | 156 | 5.6 | 255 | Present invention |
| 1-22 | ET-198 | 2 | 430 | 4.7 | 121 | 6.8 | 153 | Present invention |
| 1-23 | ET-199 | 2 | 501 | 4.0 | 143 | 5.7 | 203 | Present invention |
| 1-24 | ET-200 | 1 | 699 | 1.4 | 127 | 7.6 | 152 | Present invention |
| 1-25 | ET-201 | 2n | (408)n | 4.9 | 141 | 5.7 | 193 | Present invention |
| 1-26 | ET-216 | 6n | (703)n | 8.5 | 153 | 6.2 | 147 | Present invention |

As presented in Table 1, it has been found that the injection properties of a charge is improved by using a material containing a nitrogen-containing atom having a lone pair of electrons that does not participate in aromaticity. In particular, those which have the content ratio [n/M] of effective lone pair of electrons in a range of from 5.0×10⁻³ to 1.0×10⁻² are able to achieve a low driving voltage even in a reversely layered constitution.

In addition, it has been indicated that a compound having a specific structure (structure of Formula (5)) has a favorable luminous efficiency and a favorable half-life.

Incidentally, for the compounds exemplified as the specific examples of an organic compound having a nitrogen atom, the number n of effective lone pair of electrons and the content ratio [n/M] of effective lone pair of electrons can be determined in the same manner as in the compound presented in Table 1.

In addition, there is a possibility of obtaining an element having a sequentially layered constitution but the same lifespan in a case in which the sputtering technique is progressed and the film formation conditions of an electride having low energy and little damage is established in the future.

### Example 2

### Fabrication of Reversely Layered Type Blue Phosphorescent Organic EL Element: n-Doped ETL Type

(1) Fabrication of Organic EL Element 2-1

In the fabrication of the organic EL element 1-1 of Example 1, Alq₃ and metal lithium as the electron transport layer were co-deposited on the electron injection layer composed of C12A7 at a deposition rate of 0.1 nm/sec and 0.006 nm/sec, respectively, to form an n-doped electron transport layer having a layer thickness of 100 nm.

Subsequently, the organic EL element 2-1 was fabricated in the same manner as in the organic EL element 1-1 except that an electron transport layer composed of only Alq₃ was co-deposited on the n-doped electron transport layer at a deposition rate of 0.1 nm/sec in the same manner to form an electron transport layer having a layer thickness of 10 nm.

(2) Fabrication of Organic EL Elements 2-3, 2-5, 2-8, 2-15, 2-18, 2-19, and 2-22

The organic EL elements 2-3, 2-5, 2-8, 2-15, 2-18, 2-19, and 2-22 having an n-doped electron transport layer were fabricated by replacing Alq₃ used in the organic EL element 2-1 with the respective electron transporting materials and subjected to the same evaluation as in Example 1.

TABLE 2

| | | | | Content | | | | |
|---|---|---|---|---|---|---|---|---|
| | | | | ratio of | | | | |
| | | Number of | | effective | | | | |
| | | effective | | lone pair of | Luminous | Initial | | |
| Organic | Electron | lone pair of | Molecular | electrons | efficiency | driving | Half-life | |
| EL | transporting | electrons | weight | (×10⁻³) | (relative | voltage | (relative | |
| element | material | [n] | [M] | [n/M] | value) | (V) | value) | Remarks |
| 1-1 | Comparative | 0 | 459 | 0 | 100 | 9.1 | 100 | Comparative |
| | Compound 1 | | | | | | | Example |
| | | | | | | | | (non-doped) |
| 2-1 | Comparative | 0 | 459 | 0 | 122 | 7.3 | 64 | Comparative |
| | Compound 1 | | | | | | | Example |
| 1-3 | ET-10 | 4 | 717 | 5.6 | 166 | 4.8 | 326 | Present |
| | | | | | | | | invention |
| | | | | | | | | (non-doped) |
| 2-3 | ET-10 | 4 | 717 | 5.6 | 245 | 3.9 | 471 | Present |
| | | | | | | | | invention |
| 1-5 | ET-113 | 6 | 617 | 9.7 | 138 | 5.9 | 186 | Present |
| | | | | | | | | invention |
| | | | | | | | | (non-doped) |
| 2-5 | ET-113 | 6 | 617 | 9.7 | 155 | 4.4 | 228 | Present |
| | | | | | | | | invention |
| 1-8 | ET-127 | 4 | 517 | 7.7 | 142 | 5.7 | 214 | Present |
| | | | | | | | | invention |
| | | | | | | | | (non-doped) |
| 2-8 | ET-127 | 4 | 517 | 7.7 | 212 | 4.4 | 253 | Present |
| | | | | | | | | invention |
| 1-15 | ET-158 | 4 | 578 | 6.9 | 154 | 5.0 | 273 | Present |
| | | | | | | | | invention |
| | | | | | | | | (non-doped) |
| 2-15 | ET-158 | 4 | 578 | 6.9 | 188 | 4.1 | 371 | Present |
| | | | | | | | | invention |
| 1-18 | ET-175 | 4 | 673 | 5.9 | 154 | 6.1 | 190 | Present |
| | | | | | | | | invention |
| | | | | | | | | (non-doped) |
| 2-18 | ET-175 | 4 | 673 | 5.9 | 177 | 4.8 | 236 | Present |
| | | | | | | | | invention |
| 1-19 | ET-184 | 4 | 552 | 7.2 | 129 | 6.5 | 174 | Present |
| | | | | | | | | invention |
| | | | | | | | | (non-doped) |
| 2-19 | ET-184 | 4 | 552 | 7.2 | 148 | 5.7 | 196 | Present |
| | | | | | | | | invention |
| 1-22 | ET-198 | 2 | 430 | 4.7 | 121 | 6.8 | 153 | Present |
| | | | | | | | | invention |
| | | | | | | | | (non-doped) |
| 2-22 | ET-198 | 2 | 430 | 4.7 | 154 | 5.5 | 155 | Present |
| | | | | | | | | invention |

As presented in Table 2, it has been found that the organic EL element of the present invention is superior to the organic EL element of Comparative Example in the luminous efficiency, the initial driving voltage, and the half-life. In addition, it has been found that the organic EL element is able to significantly decrease the initial driving voltage as compared to the element used in Example 1. In addition, it has been found that it is possible to improve the initial driving voltage and the luminous efficiency while maintaining the half-life at that time.

### Example 3

### Fabrication of Green Phosphorescent Organic EL Element Having Sequentially Layered Constitution

(1) Fabrication of Organic EL Element 3-1

The ITO substrate which was patterned and cleaned in the same manner as in Example 1 was set in a vacuum deposition apparatus, and the materials constituting the respective layers were filled in the respective crucibles for vapor deposition in the respective optimum amounts for the fabrication of the element. One that was fabricated with a material for resistance heating made of molybdenum or tungsten was used as the crucible for vapor deposition.

After the pressure of the vacuum deposition apparatus was decreased to a vacuum degree of 1×10⁻⁴ Pa, and HAT was heated by energizing the crucible for vapor deposition containing HAT and deposited on the ITO transparent electrode at a deposition rate of 0.1 nm/sec to form a hole injection layer having a layer thickness of 20 nm.

Subsequently, α-NPD was deposited thereon in the same manner to form a hole transport layer having a layer thickness of 20 nm.

Subsequently, CBP and GD-1 were co-deposited thereon at a deposition rate of 0.1 nm/sec and 0.0064 nm/sec, respectively, to form a first luminous layer having a layer thickness of 40 nm.

Subsequently, BAlq was deposited thereon in the same manner to form a hole blocking layer having a layer thickness of 10 nm.

Thereafter, Alq3 was deposited thereon at a deposition rate of 0.1 nm/sec to form an electron transport layer having a layer thickness of 30 nm.

Subsequently, this element was transferred to a sputtering apparatus without being exposed to the air, and a C12A7 thin film was formed thereon by sputtering in 10 nm. As the sputtering conditions, the atmosphere was an argon gas at 500 mPa, the substrate temperature was room temperature, and the input power was 100 W.

The resultant element was returned to the deposition chamber again without being exposed to the air, and aluminum was deposited thereon to form a negative electrode having a film thickness of 110 nm, thereby fabricating the organic EL element 3-1. The evaluation of the respective organic EL elements was conducted in the same manner as in Example 1. Incidentally, the substrate temperature at the time of the deposition was room temperature (25° C.).

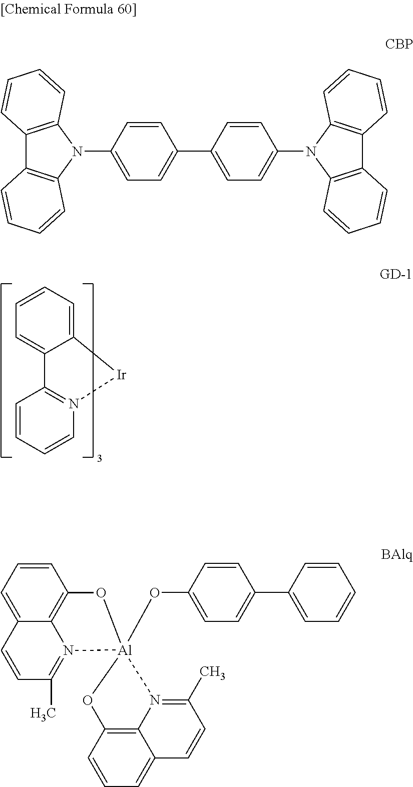

(2) Fabrication of Organic EL Elements 3-3, 3-5, 3-8, 3-15, 3-18, 3-19, and 3-22

The organic EL elements 3-3, 3-5, 3-8, 3-15, 3-18, 3-19, and 3-22 having an electron transport layer were fabricated in the same manner as in the organic EL element 3-1 and subjected to the same evaluation as in Example 1.

TABLE 3

| | | | | Content | | | | |
|---|---|---|---|---|---|---|---|---|
| | | | | ratio of | | | | |
| | | Number of | | effective | | | | |
| | | effective | | lone pair of | Luminous | Initial | | |
| Organic | Electron | lone pair of | Molecular | electrons | efficiency | driving | Half-life | |
| EL | transporting | electrons | weight | (×10⁻³) | (relative | voltage | (relative | |
| element | material | [n] | [M] | [n/M] | value) | (V) | value) | Remarks |
| 3-1 | Comparative | 0 | 459 | 0 | 100 | 7.5 | 100 | Comparative |
| | Compound 1 | | | | | | | Example |
| 3-3 | ET-10 | 4 | 717 | 5.6 | 144 | 3.9 | 192 | Present invention |
| 3-5 | ET-113 | 6 | 617 | 9.7 | 122 | 4.8 | 141 | Present invention |
| 3-8 | ET-127 | 4 | 517 | 7.7 | 124 | 4.4 | 166 | Present invention |
| 3-15 | ET-158 | 4 | 578 | 6.9 | 138 | 4.2 | 173 | Present invention |
| 3-18 | ET-175 | 4 | 673 | 5.9 | 121 | 6.1 | 148 | Present invention |
| 3-19 | ET-184 | 4 | 552 | 7.2 | 119 | 5.6 | 174 | Present invention |
| 3-22 | ET-198 | 2 | 430 | 4.7 | 117 | 6.1 | 133 | Present invention |

As presented in Table 3, it has been found that the organic EL element of the present invention has a higher luminous efficiency than the organic EL element of Comparative Example, and the organic EL element of the present invention is superior to the organic EL element of Comparative Example in the initial driving voltage and the half-life. In other words, it has been confirmed that the combination of the electron transport layer with the electron injection layer is useful even in an organic EL element having a sequentially layered constitution that has been general so far.

### Example 4

### Fabrication 1 of Reversely Layered Type White Phosphorescent Organic EL Element

(1) Fabrication of Organic EL Element 4-1

A substrate (NA45 manufactured by NH Techno Glass Corp.) obtained by forming a film of ITO as the negative electrode on a glass substrate of 100 mm×100 mm×1.1 mm in 100 nm was patterned. Thereafter, this transparent substrate provided with an ITO transparent electrode was subjected to the ultrasonic cleaning with isopropyl alcohol, dried with a dry nitrogen gas, and subjected to the UV ozone cleaning for 5 minutes.

This transparent substrate was fixed to the substrate holder of a commercially available RF sputtering apparatus. An n-type amorphous oxide semiconductor obtained by molding C12A7 on a flat plate with reference to the method described in JP 2013-40088 A was used as the sputtering target.

For the sputtering, a film was formed in an atmosphere of argon gas at 500 mPa, at a substrate temperature of room temperature and an input power of 100 W, thereby obtaining an electron injection layer composed of a C12A7 thin film having a film thickness of 10 nm.

Incidentally, it has been confirmed that the C12A7 thin film is a C12A7 film which is in an amorphous state and has a broad spectrum by the XRD measurement of the C12A7 thin film for analysis formed at the same time.

Subsequently, the electron injection layer was transferred to a vacuum deposition apparatus without being exposed to the air and fixed to the substrate holder of the vacuum deposition apparatus.

The materials constituting the respective layers were filled in the respective crucibles for vapor deposition in the vacuum deposition apparatus in the respective optimum amounts for the fabrication of the element. One that was fabricated with a material for resistance heating made of molybdenum or tungsten was used as the crucible for vapor deposition.

After the pressure of the vacuum deposition apparatus was decreased to a vacuum degree of 1×10⁻⁴ Pa, and ET-10 was heated by energizing the crucible for vapor deposition containing ET-10 and deposited on the electron injection layer composed of C12A7 at a deposition rate of 0.1 nm/sec, thereby forming an electron transport layer having a layer thickness of 45 nm.

Subsequently, ET-127 was deposited thereon at a deposition rate of 0.1 nm/sec to form a hole blocking layer having a layer thickness of 4.0 nm.

Subsequently, H-1 and BD-1 were co-deposited thereon at a deposition rate of 0.09 nm/sec and 0.01 nm/sec, respectively, to form a first luminous layer having a layer thickness of 15 nm.

Subsequently, H-1, GD-1, and RD-1 were co-deposited thereon at a deposition rate of 0.088 nm/sec, 0.01 nm/sec, and 0.002 nm/sec, respectively, to form a second luminous layer having a layer thickness of 10 nm.

Subsequently, HTD-1 was deposited thereon at a deposition rate of 0.1 nm/sec to form a hole transport layer having a layer thickness of 70 nm.

Thereafter, HAT was deposited thereon at a deposition rate of 0.1 nm/sec to form a hole injection layer having a layer thickness of 10 nm.

Furthermore, aluminum was deposited thereon in 100 nm to form a positive electrode.

The nonluminescent surface side of the element was covered with a can-shaped glass case in an atmosphere of highly pure nitrogen gas having a purity of 99.999% or higher, the electrode lead-out wiring was installed thereto, and a light extraction sheet is adhered to the glass surface, thereby fabricating the organic EL element 4-1.

Incidentally, the compounds which are newly used in the present Example are those having the following Chemical Structural Formulas.

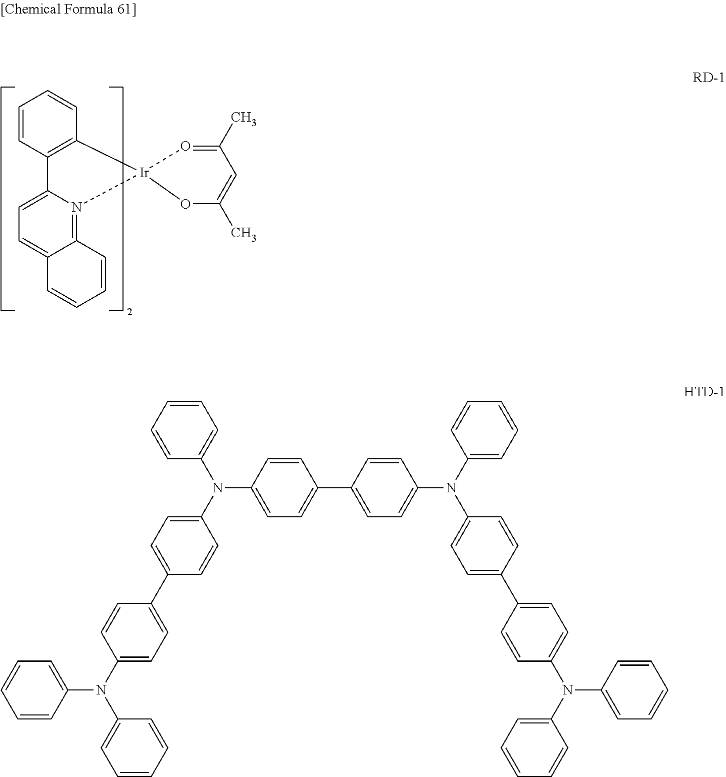

It has been confirmed that luminescence of white (CIEx, y=0.45, 0.41) at 1000 cd/m² is obtained from the organic EL element 4-1 thus obtained at an applied voltage of 4.5 V.

### Example 5

### Fabrication 2 of Reversely Layered Type White Phosphorescent Organic EL Element

(1) Fabrication of Organic EL Element 5-1

The organic EL element 5-1 was formed in the same manner except that an electron transport layer having the following bilayer constitution was formed on the electron injection layer composed of C12A7 in the fabrication of the reversely layered type white phosphorescent organic EL element of Example 4.

The electron transport layer was formed on the electron injection layer composed of C12A7 by spin coating under the following conditions in a glove box under the following conditions.

<Electron Transport Layer Coating Solution of EL Element 5-1>

ET-216: 3 mg

Dehydrated 1,1,1,3,3,3-hexafluoroisopropanol: 3 ml

The dissolved solution was formed into a film by a spin coating method under conditions of 1000 rpm and 30 seconds, and the film was dried by heating at 120° C. for 1 hour in a glove box, thereby providing an electron transport layer having a layer thickness of 5 nm.

Thereafter, the electron transport layer was transferred to a vacuum deposition apparatus, and ET-10 was deposited thereon in 15 nm, thereby forming a stacked type electron transport layer.

The organic EL element 5-1 was obtained by forming a hole blocking layer, a first luminous layer, a second luminous layer, a hole transport layer, a hole injection layer, and a positive electrode in the same manner as in the fabrication of the organic EL element 4-1 thereafter.

It has been confirmed that luminescence of white (CIE x, y=0.46, 0.42) at 1000 cd/m² is obtained from the organic EL element 5-1 thus obtained at an applied voltage of 4.2 V.

### REFERENCE SIGNS LIST

1 Transparent electrode
3 Organic layer
3a Hole injection layer
3b Hole transport layer
3c Luminous layer
3d Electron transport layer
3e Electron injection layer
5a, 5b, 5c, and 5d Counter electrode
11 Substrate
13 and 131 Transparent substrate (substrate)
13a and 131a Light extracting surface
15 Auxiliary electrode
17 Sealing material
19 Adhesive
100, 200, 300, and 400 Organic EL element
101 Organic EL element in lighting device
102 Glass cover
105 Counter electrode
106 Organic layer
107 Glass substrate with transparent electrode
108 Nitrogen gas
109 Water trapping agent

1. An organic electroluminescent element comprising at least an electron injection layer, an electron transport layer, and a luminous layer between a positive electrode and a negative electrode, wherein
the electron injection layer contains an electride,
the electron transport layer contains an organic compound having a nitrogen atom,
at least one of the nitrogen atoms has a lone pair of electrons that does not participate in aromaticity, and
the lone pair of electrons does not coordinate a metal.

2. The organic electroluminescent element according to claim 1, wherein the electron injection layer contains at least 12CaO.7Al₂O₃ as the electride.

3. The organic electroluminescent element according to claim 1, wherein a content ratio [n/M] of effective lone pair of electrons is in a range of from 4.0×10⁻³ to 2.0×10⁻² when the number of the lone pair of electrons is denoted as the number n of effective lone pair of electrons and a molecular weight of the organic compound is denoted as M.

4. The organic electroluminescent element according to claim 1, wherein the organic compound is a low molecular weight compound having a structure represented by the following Formula (1), a polymer compound having a structural unit represented by the following Formula (2), or a polymer compound having a structural unit represented by the following Formula (3):
[Chemical Formula 1]
(A₁)ₙ₁-y₁ Formula (1)
[in Formula (1), A₁ represents a monovalent nitrogen atom-containing group. n1 represents an integer of 2 or more. A plurality of A₁ may be the same as or different from one another. y₁ represents a n1-valent linking group or a single bond.] 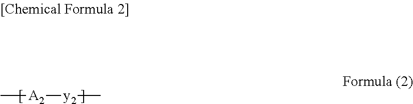
[in Formula (2), A₂ is a divalent nitrogen atom-containing group. y₂ represents a divalent linking group or a single bond.] 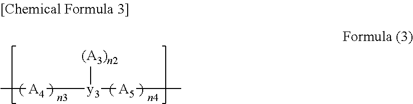
[in Formula (3), A₃ represents a monovalent nitrogen atom-containing group. A₄ and A₅ each independently represent a divalent nitrogen atom-containing group. n2 represents an integer of 1 or more, and n3 and n4 each independently represent an integer of 0 or 1. y₃ represents a (n2+2)-valent linking group.].

5. The organic electroluminescent element according to claim 4, wherein the organic compound is a low molecular weight compound represented by Formula (1) above.

6. The organic electroluminescent element according to claim 4, wherein the organic compound contains a pyridine ring in its chemical structure.

7. The organic electroluminescent element according to claim 4, wherein the organic compound has a structure represented by the following Formula (4): 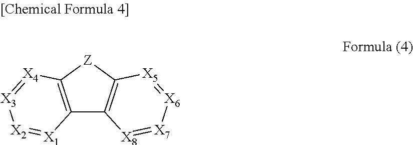
[in Formula (4), Z represents CR₁R₂, NR₃, O, S, PR₄, P(O)R₅, or SiR₆R₇. X₁ to X₈ represent CR₈ or N, and at least one of them represents N. R₁ to R₈ each independently represent a single bond, a hydrogen atom, a substituted or unsubstituted alkyl group having from 1 to 20 carbon atoms, a substituted or unsubstituted cycloalkyl group having from 3 to 20 carbon atoms, a substituted or unsubstituted aryl group having from 6 to 30 carbon atoms, a substituted or unsubstituted heteroaryl group having from 1 to 30 carbon atoms, or a substituted or unsubstituted alkyloxy group having from 1 to 20 carbon atoms.].

8. The organic electroluminescent element according to claim 7, wherein X₃ or X₄ in Formula (4) above represents a nitrogen atom.

9. The organic electroluminescent element according to claim 4, wherein the organic compound has a structure represented by the following Formula (5): 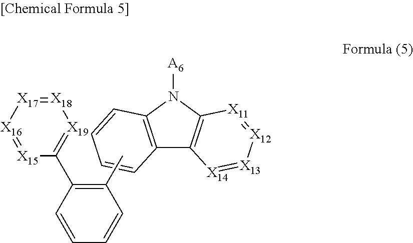
[in Formula (5), A₆ represents a substituent. X₁₁ to X₁₉ each represent C(R₂₁) or N. R₂₁ represents a hydrogen atom or a substituent. Provided that, at least one of X₁₅ to X₁₉ represents N.].

10. The organic electroluminescent element according to claim 1, wherein
the negative electrode is a transparent electrode, and
the organic electroluminescent element comprises an electron injection layer, an electron transport layer, a luminous layer, a hole transport layer, and a positive electrode on the negative electrode in this order.

11. The organic electroluminescent element according to claim 1, wherein the organic compound contains an electron donating dopant.

12. A display device comprising the organic electroluminescent element according to claim 1.

13. A lighting device comprising the organic electroluminescent element according to claim 1.

14. The organic electroluminescent element according to claim 2, wherein a content ratio [n/M] of effective lone pair of electrons is in a range of from 4.0×10⁻³ to 2.0×10⁻² when the number of the lone pair of electrons is denoted as the number n of effective lone pair of electrons and a molecular weight of the organic compound is denoted as M.

15. The organic electroluminescent element according to claim 2, wherein the organic compound is a low molecular weight compound having a structure represented by the following Formula (1), a polymer compound having a structural unit represented by the following Formula (2), or a polymer compound having a structural unit represented by the following Formula (3):
[Chemical Formula 1]
(A₁)ₙ₁-y₁ Formula (1)
[in Formula (1), A₁ represents a monovalent nitrogen atom-containing group. n1 represents an integer of 2 or more. A plurality of A₁ may be the same as or different from one another. y₁ represents a n1-valent linking group or a single bond.] 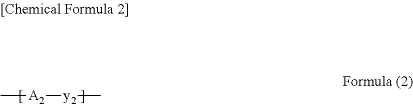
[in Formula (2), A₂ is a divalent nitrogen atom-containing group. y₂ represents a divalent linking group or a single bond.] 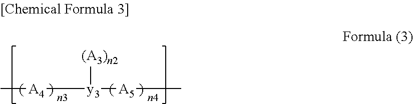
[in Formula (3), A₃ represents a monovalent nitrogen atom-containing group. A₄ and A₅ each independently represent a divalent nitrogen atom-containing group. n2 represents an integer of 1 or more, and n3 and n4 each independently represent an integer of 0 or 1. y₃ represents a (n2+2)-valent linking group.].

16. The organic electroluminescent element according to claim 2, wherein
the negative electrode is a transparent electrode, and
the organic electroluminescent element comprises an electron injection layer, an electron transport layer, a luminous layer, a hole transport layer, and a positive electrode on the negative electrode in this order.

17. The organic electroluminescent element according to claim 2, wherein the organic compound contains an electron donating dopant.

18. A display device comprising the organic electroluminescent element according to claim 2.

19. A lighting device comprising the organic electroluminescent element according to claim 2.

20. The organic electroluminescent element according to claim 3, wherein the organic compound is a low molecular weight compound having a structure represented by the following Formula (1), a polymer compound having a structural unit represented by the following Formula (2), or a polymer compound having a structural unit represented by the following Formula (3):
[Chemical Formula 1]
(A₁)ₙ₁-y₁ Formula (1)
[in Formula (1), A₁ represents a monovalent nitrogen atom-containing group. n1 represents an integer of 2 or more. A plurality of A₁ may be the same as or different from one another. y₁ represents a n1-valent linking group or a single bond.] 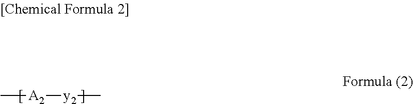
[in Formula (2), A₂ is a divalent nitrogen atom-containing group. y₂ represents a divalent linking group or a single bond.] 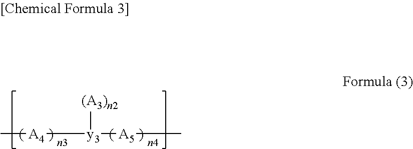
[in Formula (3), A₃ represents a monovalent nitrogen atom-containing group. A₄ and A₅ each independently represent a divalent nitrogen atom-containing group. n2 represents an integer of 1 or more, and n3 and n4 each independently represent an integer of 0 or 1. y₃ represents a (n2+2)-valent linking group.].
